(19) United States
(12) Patent Application Publication
(10) Pub. No.: US 2017/0252403 A1
(43) Pub. Date: Sep. 7, 2017

(54) METHODS AND COMPOSITIONS FOR TREATING RETINAL DISORDERS

(71) Applicant: AMARANTUS BIOSCIENCE HOLDINGS, INC., San Francisco, CA (US)

(72) Inventor: Roman URFER, Belmont, CA (US)

(21) Appl. No.: 15/517,494

(22) PCT Filed: Oct. 6, 2015
(86) PCT No.: PCT/US2015/054331

§ 371 Date: Apr. 6, 2017

Related U.S. Application Data
(60) Provisional application No. 62/060,199, filed on Oct. 6, 2014.

(51) Int. Cl. A61K 38/18; A61K 9/00; C07K 14/475

(52) U.S. Cl. CPC A61K 38/185; C07K 14/475; A61K 9/0048; A61K 9/0051; A61K 9/0019; A61K 2300/00

(57) ABSTRACT

Provided are methods and compositions for protecting treating ischemic events in the retina. The methods include administering a MANF family protein (e.g., MANF, CDNF, or fragments thereof) to a subject and performing another treatment to resolve the blockage underlying the ischemic event. The methods also include administering a MANF family protein to extend the therapeutic window for treatment of a retinal artery occlusion.

### CROSS-REFERENCE

This application claims the benefit of U.S. Provisional Application No. 62/060,199, filed Oct. 6, 2014, which application is incorporated herein by reference in its entirety.

### BACKGROUND OF THE INVENTION

Retinal ischemia can be a common cause of visual impairment and blindness. A number of clinical conditions, including central retinal artery or vein occlusion (CRAO, CRVO), diabetes, or glaucoma can make themselves manifest by a reduction of retinal blood supply. Retinal ischemia initiates a self-reinforcing destructive cascade involving neuronal depolarization, calcium influx and oxidative stress initiated by energy failure and increased glutamatergic stimulation. The initial ischemic insult results in cellular perturbations that continue to progress despite or perhaps because of, reperfusion of the ischemic tissue. Ultimately, the retinal ganglion cells (RGC) die via apoptosis.

There is a need in the art to develop treatments for retinal ischemia and other retinal disorders.

There can be a short therapeutic window following an ischemic event to resolve the occlusion before permanent damage to the retina occurs. There is a need in the art to develop treatments that increase the retinal tolerance time following the onset of an ischemic event.

### SUMMARY OF THE INVENTION

Disclosed herein are methods of increasing retinal tolerance time, reducing cell death during an ischemic event in the retina, reducing cell death following an ischemic event in the retina, treating an ischemic event in the retina, or a combination thereof, the methods comprising: (a) administering a dose of a pharmaceutical composition comprising an effective amount of a MANF family protein to a subject in need thereof; (b) performing a treatment to resolve a blockage causing the ischemic event.

In some embodiments, the MANF family protein is a mesencephalic astrocyte derived neurotrophic factor (MANF) protein, or a fragment thereof. In some embodiments, the MANF family protein comprises a sequence that has at least about 80% identity with SEQ ID NO:3. In some embodiments, the MANF family protein comprises a sequence that has 95% identity with SEQ ID NO:3.

In some embodiments, the MANF family protein is a conserved dopamine neurotrophic factor (CDNF) protein, or a fragment thereof. In some embodiments, the MANE family protein comprises a sequence that has at least about 80% identity with SEQ ID NO:6. In some embodiments, the MANF family protein comprises a sequence that has 95% identity with SEQ ID NO:6.

In some embodiments, the pharmaceutical composition is administered to an eye of the subject. In some embodiments, the pharmaceutical composition is administered by topical administration, intravitreal injection, intracameral administration, subconjunctival administration, subtenon administration, retrobulbar administration, posterior juxtascleral administration, or a combination thereof. In some embodiments, the pharmaceutical composition is administered by intravitreal injection.

In some embodiments, the dose has a volume of about 25 μl to about 150 μL.

In some embodiments, the dose has a concentration of the MANF family protein that is from about 1 mg/mL to about 20 mg/mL.

In some embodiments, the dose has a concentration of the MANF family protein that is from about 2.7 mg/mL to about 5.4 mg/mL.

In some embodiments, the effective amount of the MANF family protein is from about 50 μg to about 1000 μg. In some embodiments, the effective amount of the MANF family protein is from about 250 μg to about 300 μg.

In some embodiments, the dose is administered once every 2 to 8 weeks. In some embodiments, the dose is administered once every 2 to 4 hours. In some embodiments, the dose is only administered once.

In some embodiments, the ischemic event is a retinal artery occlusion. In some embodiments, the ischemic event is an acute retinal artery occlusion.

In some embodiments, the treatment to resolve the blockage comprises administration of a vasodilator. In some embodiments, the treatment to resolve the blockage comprises ocular massage, intravenous acetazolamide, intravenous mannitol, topical antiglaucoma drops, anterior chamber paracentisis, or a combination thereof. In some embodiments, the treatment to resolve the blockage comprises intravenous nethylprednisolone. In some embodiments, the treatment to resolve the blockage comprises Nd YAG laser treatment, pars plana vitrectonmy, or a combination thereof. In some embodiments, the treatment to resolve the blockage comprises intravenous tissue plasminogen activator, intra-arterial tissue plasminogen activator, or a combination thereof. In some embodiments, the treatment to resolve the blockage comprises panretinal photocoagulation. In some embodiments, the treatment to resolve the blockage comprises administration of a steroid.

Some embodiments further comprise diagnosing the ischemic event.

Also disclosed herein are methods of increasing retinal tolerance time, reducing cell death during a retinal artery occlusion, reducing cell death following a retinal artery occlusion, treating a retinal artery occlusion, or a combination thereof, the methods comprising administering a dose of a pharmaceutical composition comprising an effective amount of a MANF family protein to a subject exhibiting one or more symptoms of a retinal artery occlusion.

In some embodiments, the MANF family protein is a mesencephalic astrocyte derived neurotrophic factor (MANF) protein, or a fragment thereof. In some embodiments, the MANF family protein comprises a sequence that has at least about 80% identity with SEQ ID NO:3. In some embodiments, the MANF family protein comprises a sequence that has 95% identity with SEQ ID NO:3.

In some embodiments, the MANF family protein is a conserved dopamine neurotrophic factor (CDNF) protein, or a fragment thereof. In some embodiments, the MANF family protein comprises a sequence that has at least about 80% identity with SEQ ID NO:6. In some embodiments, the MANF family protein comprises a sequence that has 95% identity with SEQ ID NO:6.

In some embodiments, the pharmaceutical composition is administered to an eye of the subject. In some embodiments, the pharmaceutical composition is administered by topical administration, intravitreal injection, intracameral administration, subconjunctival administration, subtenon administration, retrobulbar administration, posterior juxtascleral administration, or a combination thereof. In some embodiments, the pharmaceutical composition is administered by intravitreal injection.

In some embodiments, the dose has a volume of about about 25 μL to about 150 μL.

In some embodiments, the dose has a concentration of the MANF family protein that is from about 1 mg/mL to about 20 mg/mL. In some embodiments, the dose has a concentration of the MANF family protein that is from about 2.7 mg/mL to about 5.4 mg/mL.

In some embodiments, the effective amount of the MANF family protein is from about 50 μg to about 1000 μg. In some embodiments, the effective amount of the MANF family protein is from about 250 μg to about 300 μg.

In some embodiments, the dose is administered once every 2 to 4 hours. In some embodiments, the dose is only administered once.

In some embodiments, the retinal artery occlusion is an acute retinal artery occlusion. In some embodiments, the retinal artery occlusion is a central retinal artery occlusion. In some embodiments, the retinal artery occlusion is a branch retinal artery occlusion.

Also disclosed herein are methods of treating a retinal disorder, the method comprising administering to a subject in need thereof an effective amount of a MANF family protein and another active agent.

In some embodiments, the MANF family protein and the another active agent have a synergistic effect upon retinal ganglion cell survival.

In some embodiments, the MANF family protein and the another active agent exhibit therapeutic synergy.

In some embodiments, the MANF family protein is MANF, or a fragment thereof.

In some embodiments, the MANF family protein is CDNF, or a fragment thereof.

In some embodiments, the another active agent is a prostaglandin analog, a beta-adrenergic receptor antagonist, an alpha adrenergic agonist, a miotic agent, a carbonic anhydrase inhibitor, or a combination thereof. In some embodiments, the another active agent is brimonidine or a pharmaceutical salt thereof.

In some embodiments, the retinal disorder is an acute retinal artery occlusion.

In some embodiments, the retinal disorder is a central retinal artery occlusion or a branch retinal artery occlusion.

In some embodiments, the retinal disorder is retinal ischemia.

In some embodiments, the retinal disorder is macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangioctasia, marfan syndrome, optic neuritis, photokeratitis, retinitis pigmentosa, retinopathy of prematurity, stargardt disease, usher syndrome, or any combination thereof.

In some embodiments, administration of the MANF family protein is topical, subconjunctival, intravitreal, retrobulbar, intracameral, systemic, or a combination thereof.

In some embodiments, the effective amount of the MANF family protein is at least about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

In some embodiments, the MANF family protein is administered once every 2 to 8 weeks.

In some embodiments, the MANF family protein is administered only once.

Also disclosed herein are pharmaceutical compositions comprising an amount of a MANF family protein and another active agent that is effective for treating a retinal disorder.

In some embodiments, the MANF family protein and the another active agent have a synergistic effect upon retinal ganglion cell survival.

In some embodiments, the MANF family protein and the another active agent exhibit therapeutic synergy.

In some embodiments, the MANF family protein is MANF, or a fragment thereof.

In some embodiments, the MANF family protein is CDNF, or a fragment thereof.

In some embodiments, the another active agent is a prostaglandin analog, a beta-adrenergic receptor antagonist, an alpha adrenergic agonist, a miotic agent, a carbonic anhydrase inhibitor, or a combination thereof. In some embodiments, the another active agent is brimonidine or a pharmaceutical salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

This specification controls in the event that a term defined herein conflicts with a term incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the three-dimensional structure of MANF as determined using multidimensional NMR spectroscopy. N- and C-terminal domains are connected with a flexible linker region. The MANF protein is almost completely helical within the two folded domains. The helices are numbered from α1 to α8. The N-terminal (N) and C-terminal (C) of the protein are indicated.

FIG. 2 illustrates Coomassie-stained SDS-PAGE (A) and Western Blot (B) analysis of purified human MANF protein. 2 μg of purified hrMANF was loaded per lane. Line 1. hrMANF; Line 2. Prestained Protein Ladder, Naxo, 8003. For Western Blot testing, anti-MANF polyclonal antibody (Icosagen AS Cat. No 310-100) was used.

FIG. 3 illustrates right eye b-wave amplitudes seven (7) days after optic nerve clamping, normalized to baseline. The treatment conditions were, from left to right, MANF 0.15 mg/mL, MANF 0.5 mg/mL, MANF 1.5 mg/mL, PBS, or Alphagan®; ** p<0.05 vs PBS (ANOVA, followed by Dunn's multiple comparisons test against PBS control); N=10 (Alphagan), N=11 (PBS), N==11 (MANF 0.15 mg/mL), N=12 (MANF 0.5 mg/ml), N:=12 (MANF 1.5 mg/mL).

FIG. 4 illustrates surviving retinal ganglion cell density seven (7) days after optic nerve clamping. The treatment conditions were, from left to right, MANF 0.15 mg/mL, MANF 0.5 mg/mL, MANF 1.5 mg/mL, PBS, or Alphagan®; * p<0.05 vs. PBS (ANOVA, followed by Dunn's multiple comparisons test against PBS control). N=11 (Alphagan, PBS), N=12 (MANF 0.15 mg/mL, MANF 0.5 mg/mL, MANF 1.5 mg/mL).

### DETAILED DESCRIPTION OF THE INVENTION

Retinal ischemia can cause visual impairment or blindness. A number of clinical conditions, including central retinal artery or vein occlusion (CRAO, CRVO), diabetes, or glaucoma make themselves manifest by a reduction of retinal blood supply. Retinal ischemia can initiate a self-reinforcing destructive cascade involving neuronal depolarization, calcium influx and oxidative stress initiated by energy failure and increased glutamatergic stimulation. The initial ischemic insult can result in cellular perturbations that continue to progress despite or perhaps because of, reperfusion of the ischemic tissue. Ultimately, the retinal ganglion cells (RGC) die via apoptosis.

Mesencephalic astrocyte-derived neurotrophic factor (MANF) and conserved dopamine neurotrophic factor (CDNF) are two known members of a novel evolutionarily conserved protein family with neurotrophic capabilities, the MANF family proteins. The first member of the family, MANF, was identified from the conditional medium of a rat type-1 astrocyte cell line, namely, the ventral mesencephalic cell line 1 (VMCL1), as a factor that promotes the survival of cultured embryonic dopaminergic neurons. MANF can also reduce infarction in the ischemic cortex in a rat model of stroke and promote the survival of cultured heart muscle cells. CDNF, on the other hand, was first identified in silico and then biochemically characterized. Structural analysis showed that both MANF and CDNF have an N-terminal saposin-like lipid-binding domain and a C-terminal domain that may be responsible for the endoplasmic reticulum (ER) stress response.

Provided are methods and compositions for protecting, stimulating the growth of, or regenerating retinal neurons and for treating retinal disorders comprising administering an effective amount of a MANF family protein and another active agent to a subject in need thereof. Retinal neurons can include visual cells (e.g., rod or cone cells), bipolar cells, ganglion cells (e.g., retinal ganglion cells), amacrine cells, horizontal cells, or any combination thereof.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting of the invention. As used herein, the indefinite articles “a”, “an” and “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

As used herein, the term “about” means plus or minus 10% of the indicated value. For example, about 100 means from 90 to 110.

All genes and gene products (including RNA and proteins), and their respective names, disclosed herein are intended to correspond to homologs from any species for which the compositions and methods disclosed herein are applicable. When a gene or gene product from a particular species is disclosed, it is understood that this disclosure is intended to be exemplary only and is not to be interpreted as a limitation unless the context in which it appears clearly indicates otherwise. For example, the genes and gene products disclosed herein, which in some embodiments relate to mammalian (including human) nucleic acid and/or amino acid sequences, are intended to encompass homologous and/or orthologous and/or paralogous genes and gene products from other animals including, but not limited to, other mammals, fish, reptiles, amphibians, birds, and other vertebrates.

As used herein, the terms “polypeptide,” “peptide,” and “protein” are equivalent and mutually interchangeable. They refer to any amino acid chain, including native peptides, degradation products, synthetically synthesized peptides, or recombinant peptides; and include any post-translational modifications thereto (for example phosphorylation or glycosylation). Polypeptides include modified peptides, which may have, for example, modifications rendering the peptides more stable or less immunogenic. Such modifications can include, but are not limited to, cyclization, N-terminus modification, C-terminus modification, peptide bond modification, backbone modification and residue modification. Acetylation—amidation of the termini of the peptide (e.g., N-terminal acetylation and C-terminal amidation) can increase the stability and cell permeability of the peptides.

As used herein, the term “fragment” refers to a portion of a compound. For example, when referring to a protein, a fragment is a plurality of consecutive amino acids comprising less than the entire length of the polypeptide.

The disclosure of a particular sequence should be understood as disclosure of all fragments of a sequence. A fragment of a sequence can be defined according to a percent length of a reference sequence (e.g., a reference protein or peptide sequence). For example, a fragment of a sequence (e.g., protein or peptide sequence) can have a length that is at least about 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the length of the reference sequence. In another example, a fragment of a sequence (e.g., protein or peptide sequence) can have a length that is at most about 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the length of the reference sequence. In another example, a fragment of a sequence (e.g., protein or peptide sequence) can have a length that is about 1-99%, 2-99%, 5-99%, 10-99%, 20-99%, 30-99%, 40-99%, 50-99%, 60-99%, 70-99%, 80-99%, 90-99%, 2-90%, 5-90%, 10-90%, 20-90%, 30-90%, 40-90%, 50-90%, 60-90%, 70-90%, 80-90%, 5-80%, 10-80%, 20-80%, 30-80%, 40-80%, 50-80%, 60-80%, 70-80%, 10-70%, 20-70%, 30-70%, 40-70%, 50-70%, 60-70%, 20-60%, 30-60%, 40-60%, 50-60%, 30-50%, 40-50%, or 30-40% of the length of the reference sequence. Fragments can also be defined as have a percent identity to a reference sequence; for example a fragment can have length that is less than the reference sequence and a percent identity of the reference sequence.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G, eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SUM J. Applied Math. 48: 1073.

The disclosure of any particular sequence herein should be interpreted as the disclosure of all sequences sharing a percent identity with the sequence. A sequence can be defined herein according to a percent identity with a reference sequence. For example, the sequence can have at least about: 50%, 55%, 60%, 65%, 70%, 75%, 80%, 85%, 90%, 95%, 96%, 97%, 98%, 99%, or 100% identity with the reference sequence. In another example, the sequence can have about: 50-60%, 50-75%, 50-80%, 50-85%, 50-90%, 50-95%, 50-97%, 50-99%, 50-100%, 60-75%, 60-80%, 60-85%, 60-90%, 60-95%, 60-97%, 60-99%, 60-100%, 75-80%, 75-85%, 75-90%, 75-95%, 75-97%, 75-99%, 75-100%, 80-85%, 80-90%, 80-95%, 80-97%, 80-99%, 80-100%, 85-90%, 85-95%, 85-97%, 85-99%, 85-100%, 90-95%, 90-97%, 90-99%, 90-100%, 95-97%, 95-99%, 95-100%, 97-99%, 97-100%, or 99-100% identity with the reference sequence. Such sequences can be called variants of the reference sequence.

A “variant” of a polypeptide comprises an amino acid sequence wherein one or more amino acid residues are inserted into, deleted from and/or substituted into the amino acid sequence relative to another polypeptide sequence. The substituted amino acid(s) can be conservative substitutions or non-conservative substitutions, depending upon the context.

Variants include fusion proteins.

Conservative substitutions are substitutions of one amino acid with a chemically similar amino acid. The following six groups each contain amino acids that are conservative substitutions for one another: (1) Alanine (A), Serine (S), Threonine (T); (2) Aspartic acid (D), Glutamic acid (E); (3) Asparagine (N), Glutamnine (Q); (4) Arginine (R), Lysine (K); (5) Isoleucine (I), Leucine (L), Methionine (M), Valine (V); and (6) Phenylalanine (F), Tyrosine (Y), Tryptophan (W).

In making changes to the peptides and proteins disclosed herein, the hydropathic index of amino acids can be considered. Each amino acid has been assigned a hydropathic index on the basis of its hydrophobicity and charge characteristics. They are: isoleucine (−4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein or peptide can be considered in designing variants of a protein or peptide. Certain amino acids can be substituted for other amino acids having a similar hydropathic index or score and still retain a similar biological activity. In making changes based upon the hydropathic index, the substitution of amino acids whose hydropathic indices are within ±2, ±1, or ±0.5 are included.

The substitution of like amino acids can also be made effectively on the basis of hydrophilicity. In certain embodiments, the greatest local average hydrophilicity of a protein or peptide, as governed by the hydrophilicity of its adjacent amino acids, correlates with a biological property of the protein or peptide.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (±0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, the substitution of amino acids whose hydrophilicity values are within ±2, ±1, ±0.5 are included.

As used herein, the term “subject” refers to any animal (e.g., mammals, birds, reptiles, amphibians, fish), including, but not limited to, humans, non-human primates, rodents, and the like, which is to be the recipient of a particular treatment. Typically, the terms “subject” and “patient” may be used interchangeably herein in reference to a subject.

As used herein, the term “administering” refers to providing a therapeutically effective amount of a chemical or biological compound or pharmaceutical composition to a subject, using intravitreal, intraocular, ocular, subretinal, intrathecal, intravenous, subcutaneous, transcutaneous, intracutaneous, intracranial, topical and the like administration. The chemical or biological compound of the present invention can be administered alone, but may be administered with other compounds, excipients, fillers, binders, carriers or other vehicles selected based upon the chosen route of administration and standard pharmaceutical practice. Administration may be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

The active ingredients (e.g., chemical or biological compound or pharmaceutical composition) disclosed herein may also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. Specifically, the pharmaceutically acceptable carriers, excipients, binders, and fillers contemplated for use in the practice of the present invention are those which render the compounds of the invention amenable to intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material may be biologically inert or lack bioactivity, such as plastic polymers, silicone, etc. and may be processed internally by the subject without affecting the effectiveness of the neurotrophic factor packaged and/or delivered therewith.

The term “effective amount,” as applied to the compound(s), biologics and pharmaceutical compositions described herein, means the quantity necessary to render the desired therapeutic result. For example, an effective amount is a level effective to treat, cure, or alleviate the symptoms of a disorder for which the therapeutic compound, biologic or composition is being administered. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

As used herein, “disorder” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities. The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like.

As used herein, “treatment” or “treating” refers to arresting or inhibiting, or attempting to arrest or inhibit, the development or progression of a disorder and/or causing, or attempting to cause, the reduction, suppression, regression, or remission of a disorder and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms. Additionally, treatment can be applied to a subject or to a cell culture.

Retinal Artery Occlusion

Acute retinal artery occlusion (RAO) represents an acute ophthalmologic emergency. A case of central retinal artery occlusion (CRAO) was first described by von Graefe in 1859.

RAO is a rare condition caused by the sudden occlusion of the central retinal artery, usually by emboli or thrombi, leading to an abrupt loss of blood supply to the inner layer of the retina and resulting in acute and often irreversible, severe vision loss. The estimated prevalence of RAO in the United States of America (USA) population is 10,450 individuals affected in 2014. Vision loss is often permanent if reperfusion of the retinal artery is not achieved within a few hours.

RAO can be classified based on where the occlusion is located. Central RAO (CRAO) affects the retinal artery at the optic nerve and accounts for 58% of RAO cases. Branch retinal artery occlusion (BRAO) results in an obstruction distal to the lamina cribrosa of the optic nerve and accounts for about 38% of RAO cases. The central retinal artery is an end artery supplying the only source of arterial blood to the inner retinal layer of the eye, the end organ. CRAO and BRAO can be readily diagnosed by direct visualization, unlike other vascular occlusive disease.

An estimated 49.5% of humans anatomically have a cilioretinal artery. The cilioretinal artery, when present, is a branch of the short posterior artery that supplies the maculopapular bundle, an area that contains the maximum amount of photoreceptors for central vision. This cilioretinal artery may become occluded (CLRAO) or it may provide some sparing effect in cases of CRAO, though the size of the area the cilioretinal artery may supply can vary from individual to individual. CLRAO is seen in about 5% of RAO cases.

RAO generally presents as acute, painless, monocular vision loss. It can be rare for RAO to occur simultaneously in both eyes, however multiple emboli have been observed in the affected eye in about ⅓rd of cases. In the case of CRAO and CLRAO, central vision loss can be quite severe. BRAO typically affects an area of the peripheral vision and may even go unnoticed by the affected individual. The incidence of RAO increases with age and can be seen more often in men compared to women. Recovery of any degree of visual acuity can depend on the removal of the occlusion within the retinal tolerance time, that is, the time before the retinal ganglion cells are irreversibly damaged. Previous studies in elderly atherosclerotic and hypertensive rhesus monkeys using central retinal artery clamping for 97, 105-120, 150-165 and ≧180 minutes, complete recovery of vision based on electroretinographic measurements and histologic findings was seen if the clamp was removed within 97 minutes, but there was increasing loss of vision and more severe histologic findings of necrosis of the inner layer of the retina the longer the occlusion lasted. This retinal tolerance time may be dependent on the duration of intracellular glycogen conversion to glucose prior to reperfusion to allow for cellular survival.

A common cause of RAO can be an embolism caused by plaque in the carotid artery. Emboli may also originate from certain diseases of the heart (e.g., aortic mitral valve lesions, tumor, myxoma, etc.), but this can be less common. These emboli have been reported to be of 3 types: 74% consisted of cholesterol, 15.5% were cholesterol and platelet fibrin, and 10.5% were calcific in nature. Rarely, acute CRAO and BRAO can be caused by various hypercoagulable states such as, for example, Protein C, Protein S, or prothrombin deficiencies; sickle cell anemia (acute sickle crisis); and anti-phospholipid antibodies. Acute CRAO and BRAO can be caused by acute and transient vasospasm (transient ischemic attack [TIA]) or by more generalized arteritis conditions, especially giant cell arteritis, which can affect the central retinal artery, though not branch retinal arteries (which are arterioles rather than arteries and thus not affected by the arteritic inflammation). Giant cell arteritis can be diagnosed as the cause of CRAO in <5% of cases, but it can be important to consider in the initial differential diagnosis as it is a treatable disease with very high dose intravenous steroid. An even less common cause of acute CRAO or BRAO may be acute serotonin release from platelet thrombi leading to vasospasm.

Many or most emboli arise from underlying cardiovascular disease. The presence of carotid artery plaque can be of greater importance than the degree of stenosis when it comes to RAO risk. Many of the embolic/thrombotic causes of CRAO and BRAO can be associated with generalized systemic disorders such as hypertension, diabetes mellitus, and tobacco smoking which can cause generalized atherosclerotic disease. Therefore, it can be important for a patient presenting with RAO to be further evaluated for other vascular risk factors and treated appropriately. Findings from a single-center, randomized audit found that 64% of patients with CRAO had at least one undiagnosed vascular risk such as hyperlipidemia (36%), hypertension (27%) and diabetes (12%) as the most prominent.

The retinal artery is the sole source of blood to the inner retina, an end organ, and acute occlusion can result in the immediate onset of symptoms. This can be unlike the case of retinal vein occlusion and chronic carotid artery/LAO hypoperfusion, where the time to onset is usually long and the symptoms can be chronic with many of the adverse end events taking a prolonged time to manifest. To achieve immediate and sufficient concentrations of mesencephalic, astrocyte-derived neurotrophic factor (MANF) to be effective, MANF can be delivered directly into the eye.

Central Retinal Artery Occlusion

The ophthalmic artery originates from the internal carotid artery. The central retinal artery is the first branch of the ophthalmic artery and supplies blood to the surface layer of the optic disc. Acute CRAO causes a sudden drop in oxygen and nutrient supply to the retina which can lead to a death of retinal ganglion cells and loss of the entire field of vision unless vascular flow is restored quickly. The retina consumes oxygen and glucose more rapidly than other tissues in the body and therefore can be at very high risk of suffering permanent damage unless the arterial vascular supply can be quickly restored. The most common location for an occlusion can be where the artery pierces the dural sheath of the optic nerve immediately posterior to the lamina cribrosa.

The inner layer of the retina can have greater sensitivity to hypoxic challenge than the outer layer of the retina. The retinal ganglion cells (RGCs) of the inner part of the retina can be sensitive to acute, transient and even mild systemic hypoxic stress. During RAO, inner retinal layer edema and pyknosis of the ganglion cell nuclei can occur very rapidly. If reperfusion is not restored quickly, RGCs can be lost through both apoptosis and necrosis and blindness can become permanent.

RGCs are the neurons that transfer visual information from the eye to the brain. The axons of the RGCs bundle to make up the optic nerve. The optic disc is the point where the optic nerve emerges from the retina and it is also the entry point of the major blood vessels that supply the retina. Retinal bipolar cells release glutamate that binds to glutamate receptors on RGCs.

When the proper threshold is reached, RGCs will depolarize. RGCs can be the only retinal cells that can produce an action potential and it is by this action potential that visual information is transmitted to the brain through the optic nerve. Under hypoxic stress, excess glutamate can be produced. Hyperexcitability of the RGCs can cause a cascade of biochemical effects, such as neuronal nitric oxide synthase (NOS) activation and increases in Ca²⁺, which have been shown to be contributing factors for RGC loss. Vision loss from RAO can result from acute loss of the arterial blood supply to the inner layer of the retina.

CRAO generally presents as acute, painless, monocular vision loss. In the case of CRAO and CLRAO, central vision loss can be quite severe. BRAG affects an area of the peripheral vision and may even go unnoticed by the affected individual. The incidence of RAO increases with age and can be seen more often in men compared to women. Recovering any degree of visual acuity can depend on the rapid removal of the occlusion in conjunction with an optimization of the retinal ischemic tolerance time.

The diagnosis of CRAO/BRAO can be a medical emergency, and can require a rapid and thorough evaluation of the underlying cause of the occlusion and institution of potential treatment to restore arterial blood flow. Identifying the source of microthrombi/emboli and immediate treatment to prevent further thrombi/emboli or dissolve intact thrombi can be indicated. Monocular and binocular CRAO and blindness also can occur with giant cell arteritis (at a reported rate of <5%) and the immediate institution of high dose corticosteroid therapy can be indicated. As a secondary part of the initial evaluation, a survey of the rest of the body can also be indicated because most thrombi/emboli arise from underlying cardiovascular disease and RAO patients are typically over 50 and are reported to have an increased mortality rate. Therefore, it can be important for a patient presenting with RAO to be evaluated for other vascular risk factors and treated appropriately while emergency treatment to restore retinal artery flow is put into place. Findings from a single-center, randomized audit found that 64% of patients with CRAO had at least one undiagnosed vascular risk such as hyperlipidaemia (36%), hypertension (27%) and diabetes (12%) as the most prominent.

RAO is an acute ophthalmologic emergency. The retinal artery is an end artery and the inner retina is an end organ because there is no other arterial supply to the inner retina. As such, RAO can be considered the ocular analogue of cerebral stroke, but without the possibility of ancillary arterial supply and backflow. As a result, in permanent CRAO, final visual acuity is be reduced to counting fingers or worse (20/200-20/400) in a reported 80% of patients. If a cilioretinal artery is present anatomically, some visual acuity may be preserved to 20/50 with only peripheral vision loss.

The time frame to significant visual loss after acute arterial occlusion call be distinctly different from that for chronic retinal vein stasis, which can result in local edema (especially of the macula), and glaucoma, all of which usually result in chronic changes to the retina.

This is in significant contradistinction to the effects of central retinal vein occlusion (CRVO). Though acute CRAO and CRVO can share a common end pathway of hypoxia and loss of nutrients, which can lead to apoptosis and death of the various retinal cell layers and ganglia, the time to blindness can be measured in hours to days with arterial occlusion as opposed to months and years with chronic retinal vein occlusion. The most common causes of chronic CRVO can be macular edema, vitreous haemorrhage, neovascularization and neovascular glaucoma.

The visual problems caused by CRVO can be slowly progressive and are often secondary to intra-ocular pathology. Treatment is often focused on the inciting pathology rather than the CRVO. While it appears that CRVO and CRAO leading to blindness have a common final pathway to retinal cell apoptosis and death, the speed to retinal ganglion death can be significantly faster with CRAO than CRVO because of the acute loss of oxygenation and nutrients from the blood that can occur in CRAO. The treatments for retinal vein occlusion (RVO) and macular oedema can include the long term use of anti-VEGF (vascular endothelial growth factor) drugs (for example, ranibizumab and aflibercept) or intraocular corticosteroids such as triamcinolone, which have not been indicated for the treatment of RAO. If there is acute combined CRAO and central retinal venous occlusion caused, for example, by physical trauma or head placement during spinal surgery procedures, the most significant problem can be the acute loss of oxygen and especially nutrients supplied to the retina as the end organ.

There is a need in the art to extend the survival of the entire retina and especially RGCs after acute RAO in that relatively short period until retinal artery flow can be restored.

Categories of Central Retinal Artery Occlusion

CRAO can be classified into 4 categories, depending on the location of the occlusion:
1) Non-arteritic permanent CRAO: This type is the most common and can account for ⅔^{rds} of CRAO cases. The occlusion is typically caused by thrombi/emboli. Vision loss can often be severe with little recovery in visual acuity.
2) Transient non-arteritic: About a reported 15-17% of CRAO cases are of this category. This type can have the best visual prognosis and, according to what was seen in animal models, is thought to be caused by transient vasospasm due to serotonin release from platelets on plaques. Treatment may not be necessary because the condition often resolves without intervention.
3) ion-arteritic CRAO with cilioretinal sparing: Perfusion of the macula region can be preserved by the cilioretinal artery, allowing for a better central visual acuity prognosis.

The size of the cilioretinal artery and the area of the macular region can vary from individual to individual. Not all individuals affected with CRAO will have a cilioretinal artery.
4) Arteritic CRAO with giant cell arteritis: This type can be less common and has been reported to include less than about 5% of CRAO cases. It can, however cause bilateral, severe and permanent vision loss. The patient can be assessed for presence of inflammatory markers and treatment with systemic corticosteroids may give the best chance of regaining visual acuity.

Diagnosis of Central Retinal Artery Occlusion

Acute CRAO can be Diagnosed Using the Following Criteria:
1. Sudden vision loss in one eye
2. Evidence of acute retinal ischemia:
   retinal opacity: the ischemic area of the retina can appear white because retinal ganglion cells can swell due to the ischemia. This whitening of the retina generally lasts about 4-6 weeks. When present, no whitening of the retina supplied by the cilioretinal artery is typically seen.
   cherry red spot: foveal center (center of the macula) can appear red because this area may not be obscured by swelling of the ganglion cells, which can allow the visualization of red blood in the choroid below.
   transient CRAO: multiple scattered patches of retinal opacity all over the posterior pole with or without intervening retina showing whitening or even a faint cherry red spot.
3. Presence of “box-carring” (“cattle trucking”) of the blood column in retinal vessels.
4. Fluorescein fundus angiography: evidence of absence or marked stasis of the retinal arterial circulation.

The presence of “box-carring” or fluorescein fundus angiography findings may not be seen in patients with transient CRAO.

Disc pallor and retinal vascular narrowing are typically seen in late stage CRAO.

Current Management of Central Retinal Artery Occlusion

One of the main goals during the initial treatment of acute CRAO and BRAO can be to relieve the ischemia as rapidly as possible. Currently available treatment options (e.g., reduction of intraocular pressure, ocular massage, use of vasodilators, hemodilution, or hyperbaric oxygen) can be used in order to dislodge the occlusion and bring more oxygen to the area. Unfortunately, many of these therapeutic maneuvers may offer limited success in comparison to natural history observations.

The use of MANF family proteins and peptides in the treatment of acute CRAO or BRAO is novel and independent of any current therapeutic maneuver. Without being limited by theory, these proteins and peptides can prolong the survival of retinal ganglion cells under stress conditions until arterial flow can be restored by the aforementioned means.

Branch Retinal Artery Occlusion

After the central retinal artery, the retinal artery has further branches that supply different quadrants of the retina. BRAO can occur when an embolus lodges in one of these distal branches, and can cause a sudden but focal loss of the field of vision.

CLRAO can be included under the general category of BRAO.

Categories of Branch Retinal Artery Occlusion

BRAO can be classified into the following categories based on the site of occlusion:
1. BRAO: BRAO can be permanent or transient. Visual prognosis following BRAO can be correlated with the site of the obstruction and the portion of the visual field affected. It has been reported that visual acuity of 20/40 or better is seen initially in 74% of permanent BRAO and 97% of transient BRAO; and finally on follow-up, in 89% and 100 respectively;
2. CLRAO: similar to CRAO, CLRAO can be further broken down into 3 distinct sub-types:
   a. Non-arteritic-CLRAO,
   b. Arteritic CLRAO associated with giant cell arteritis,
   c. CLRAO associated with central retina vein occlusion (CRVO).

Diagnosis of Branch Retinal Artery Occlusion

Acute BRAO can be diagnosed with the following criteria:
1. Sudden onset of visual deterioration;
2. Evidence of acute retinal ischemia in the distribution of the occluded branch retinal artery;
3. Evidence of retinal edema in the distribution of the affected vessel only; or
4. Fluorescein fundus angiography: evidence or absence or marked stasis of circulation in the involved branch retinal artery (except in transient BRAO).

Current Management of Branch Retinal Artery Occlusion

Permanent BRAO currently may not be treated if the perifoveolar vessels are not threatened. Transient BRAO may not require treatment, because it often does not cause noticeable visual changes and the occlusion resolves on its own. Treatments similar to those used to restore vascular flow in CRAO have been reported, but have not been shown to be more effective than natural history. Additionally, more invasive treatments to remove the emboli can carry significant complications and risk that may be out of proportion to the usually minor visual field defects.

Treatment of non-arteritic CLRAO can follow that of CRAO treatment. However, it has been reported that the available treatment options may not be better than natural history.

In a similar fashion to arteritic-CRAO, arteritic-CLRAO can be treated with systemic steroids.

Loss of Retinal Ganglion Cells

It has been reported that the inner layer of the retina consumes oxygen and glucose more rapidly than almost any other tissue and the inner layer of the retina has the greatest sensitivity to hypoxic challenges compared to the outer layer of the retina. The central retinal artery is the sole source of blood to the retinal ganglion cells (ROGCs) in the inner layer of the retina. RGCs can be highly sensitive to acute, transient and even mild local and systemic hypoxic stress. Acute occlusion of the central retinal artery can result in an acute loss of oxygen and nutrients. During RAO, inner retinal layer edema and pyknosis of the ganglion cell nuclei can be seen. If reperfusion is not restored quickly, RGCs can be lost through both apoptosis and necrosis.

RGCs are the neurons that transfer visual information from the eye to the brain. The axons of the RGCs bundle to make up the optic nerve. The optic disc is the point where the optic nerve emerges from the retina and it is also where the major blood vessels enter that supply the retina.

Biopolar retinal cells can release glutamate to bind to glutamate receptors on RGCs. When the proper threshold is reached, RGCs can depolarize. Interestingly, RGCs are reported to be the only retinal cells that can produce an action potential, and it is via this action potential that visual information can be transmitted to the brain through the optic nerve. Under hypoxic stress however, excess glutamate can be produced. Hyperexcitability of the RGCs results, which can cause a cascade of biochemical effects such as neuronal nitric oxide synthase (NOS) activation and increases in Ca²⁺. These have been shown to be contributing factors for RGC loss.

Natural History of Untreated Retinal Artery Occlusion

The prognosis for future visual acuity in acute RAO can be dependent on the duration of occlusion. The prognosis for visual acuity recovery can be good for acute CRAO if blood flow is restored expeditiously. Permanent CRAO can have a poor prognosis and little if any improvement typically occurs over time. For example, even though about 22% of cases have reported spontaneous improvements in permanent non-arteritic CRAO, less than 10% have been reported to have had any meaningful recovery of vision.

Difficulty in Diagnosis and Treatment

In a study, transient CRAO in elderly, atherosclerotic, hypertensive rhesus monkeys has been evaluated by clamping the CRA in four groups of animals for the durations of 97, 105-120, 150-165, and ≧180 mins respectively. The results of this study showed that irreversible damage begins about 97 minutes following CRAO. Additionally, the damage increased in magnitude the longer the duration of the CRAO. Based on this study and other data evaluated, the ideal therapeutic window may be less than 3 hours but up to no more than 6.5 hours in cases of complete occlusion. Therapy may work beyond 6.5 hours if the occlusion is incomplete.

Unfortunately patients are not always seen in this rather short, ideal treatment window for treatments to be the most effective in recovering visual acuity by restoring perfusion.

Additionally, in the case of BRAO, current standard treatments may not be better than the natural history of untreated BRAO. Furthermore, invasive treatments aimed at emboli removal can carry significant complications.

Debilitating Nature of Monocular Vision Loss

In permanent CRAO, reported final visual acuity for 80% of patients can be counting fingers or worse (20/200-20/400) in the affected eye. The resulting blindness in one eye can have significant impact on the patient's activities of daily living and initial quality of life following the acute monocular blindness. The brain uses kinesthetic cues arising from the convergence (binocular—eye aiming) and accommodation (focusing) to assist with orientation in space. Therefore, loss of vision in one eye can cause impairment in spatial orientation. This can have further impact on depth perception, balance, eye-hand coordination, and other visual based motor skills resulting in clumsiness, difficulty in maneuvering around objects when walking, driving, sports, and ability to do hobbies. Not surprisingly those individuals with sudden monocular vision loss can be at an increased risk of accidents in comparison to binocular sighted individuals.

These changes may render an individual unable to perform in occupations that require close work or involve vehicle operation. They may additionally have challenges or be required to give up certain hobbies and sports activities. Further, self-image may be affected.

It can be additionally important for the patient to take steps to protect the one good eye such as wearing protective eyewear.

Current Approached to Treatment

The goal of current treatment approaches can be to reperfuse the ischemic tissue as quickly as possible by removing the occlusion and then to institute secondary prevention early. Therefore management can be broken down to 3 stages. The focus of acute CRAO treatment can be to restore ocular perfusion. In the subacute stage treatment can be focused on preventing secondary neovascular glaucoma complications. Long-term management can be focused on preventing other vascular ischemic events to the eye or other end organs.

Standard treatments can include reducing intraocular pressure, ocular massage, vasodilators, hemodilution, hyperbaric oxygen, steroids, and use of anticoagulants such as heparin and aspirin. Arteritic CRAO and BRAO types can be treated with steroids.

Cases of BRAO are not usually treated since current treatment options have not proven to be better than natural history and more invasive treatments carry significant potential complications. Table 22 outlines the most common treatments for permanent non-arteritic CRAO.

TABLE 22
Current Treatment Options for Permanent Non-Arteritic CRAO

| Treatment Goal | Treatment Options |
|---|---|
| Increase blood oxygen | Vasodilators: Pentoxyphyline, inhalation |
| | of carbogen, hyperbaric oxygen, sublingual |
| | isosorbide dinitrite |
| Reduce intraocular pressure | Ocular massage |
| (increase retinal artery | Intravenous acetazolamide |
| perfusion or dislodge the | Intravenous mannitol |
| occlusion) | Topical antiglaucoma drops |
| | Anterior chamber paracentisis |
| Reduce retinal edema | Intravenous methylprednisolone |
| Lyse or dislodge clot | Nd YAG laser |
| | Pars plana vitrectomy |
| Thrombolysis of embolus | Tissue plasminogen activator (tPA) |
| | (intravenous or intra-arterial) |
| Neovascularization | Panretinal photocoagulation |

The above treatment options can also be used in non-arteritic CLRAO cases.

The currently available treatments are aimed at opening the occluded artery before irreversible damage occurs to the RGCs. The therapeutic maneuvers may not be effective and may not result in improved visual acuity above that seen in natural history studies. Part of the problem can be the long time it may take for patients to be seen after the time of onset. However, even when seen promptly, most therapy may not be highly effective. For example, the use of very aggressive anti-thrombotic therapy, such as intra-arterial injection with tPA, can have a very narrow treatment initiation window that may be missed by many patients and invasive treatments aimed at emboli removal can carry significant complications. For BRAO, current standard treatments have not been shown to be any better than natural history.

However, it is also clear that the occlusions can resolve spontaneously over time and the ability to support the prolonged survival of RGCs in the face of acute CRAO may result in the restoration of vision. Currently there is no effective neuroprotective agent available for the treatment of acute retinal ischemia. Furthermore, there is no effective treatment available to increase the retinal tolerance time. The use of MANF Family Proteins (e.g., MANF, CDNF, and fragments thereof) may offer a novel approach to this problem. MANF family proteins can also be used prophylactically.

MANF Family Proteins

Neurotrophic factors are small proteins that are synthesized and released predominantly by glial cells that induce neurons to up-regulate survival programs that help protect the cells from apoptosis. One of these neurotrophic factors, mesencephalic astrocyte-derived neurotrophic factor (MANF (NM_006010 (mRNA); NP_006001 (protein); US Pub Appln No. 20090282495)), is an 18 kDa secreted protein. Conserved dopaninergic neurotrophic factor (CDNF (NM_001029954 (mRNA); NP_001025125 (protein)) is the second member of the MANF family of proteins to be discovered.

The endoplasmic reticulum (ER) can be a key site of protein synthesis, folding, and export. Additionally, the ER can be important for intracellular calcium homeostasis and cell death signaling activation. ER quality control mechanisms monitor protein folding and can prevent the transport and secretion of immature proteins.

Misfolded proteins can be discarded by ER-associated degradation. When ER stress overwhelms the capacity of the quality control system, unfolded or misfolded proteins can accumulate in the ER. Various stimuli, including hypoxia can cause accumulation of unfolded or misfolded proteins triggering ER stress. ER stress sensor proteins can activate an intracellular signal transduction pathway called the unfolded protein response (UPR). The UPR can increase the expression of several target genes to attempt to restore ER homeostasis. The functions of UPR target genes can vary broadly, and can include protein folding helpers (e.g., chaperones); or proteins involved in glycosylation, oxidative stress response, protein trafficking, lipid biosynthesis or ER-associated degradation. However, the apoptotic signaling pathway can be initiated if the UPR is unable to restore homeostasis in order to remove an unhealthy cell.

MANF was initially described as a member of a new class of neurotrophic factors that selectively promote survival and sprouting of cultured dopaminergic neurons. Anti-apoptotic and neurotrotrophic activities of MANF have been demonstrated in vitro, in the developing brain of Drosophila and Zebrafish, in several rodent models of Parkinson's disease and ischemic brain injury, as well as in other tissues outside the central nervous system. MANF can be expressed in the retina during early post-natal development, and has been observed to peak at post-natal day 10 and then steadily decreases as the retina matures.

MANF was initially isolated from a cell-line that constitutively expressed and secreted MANF, but regulation of expression and secretion of MANF was mostly studied in the context of the cellular stress response. MANF has also been identified as an UPR target gene. In a subsequent microarray study of genes induced by the UPR, MANF was reported to be one of twelve regulated proteins. The MANE promoter contains an ER stress response element, ERSE-II, that can be activated by known ER stressors like tunicamycin and thapsigargin. Induction of MANF expression by ER stressors has been demonstrated in several independent studies in numerous cell lines.

Recombinant MANF has been reported to have fully protected primary mixed cortical/hippocampal neuronal cultures that were exposed to the ER stressor tunicamycin. This was shown by quantification of terminal deoxynucleotidyl transferase mediated dUTP nick-end labeling (TUNEL)-positive cells as a marker of apoptosis. Hence, MANF can not only be expressed in response to ER stress, but also, recombinant MANF can counteract apoptosis induced by an ER stressor.

Ischemia can lead to ER stress. MANF expression can be induced by ischemic conditions, including ischemia of the heart and the brain.

A commonly used model to investigate ischemia of the brain is transient middle-cerebral artery occlusion (tMCAO), representing the acute, transient nature of the disruption of arterial blood supply to the brain. Administration of MANF in a tMCAO model reduced infract size on day 2 (50%) and enhanced functional recovery on day 7 compared to vehicle treated animals. The anti-apoptotic effects of MANF were shown through significantly reduced TUNEL pixel density at the site proximal to MANF injection. In an experimental stroke study, MANF was expressed in the cortex using an AAV-based vector and animals were subjected to tMCAO. Infarct size was reduced by 40% and again early effects on functional recovery were observed.

Presented herein are the results of a study using MANF in an animal model of acute retinal ischemia-induced ganglion cell degeneration in the rat eye. The results presented herein support the idea that MANF can preserve inner retinal function and can protect retinal ganglion cells against apoptosis

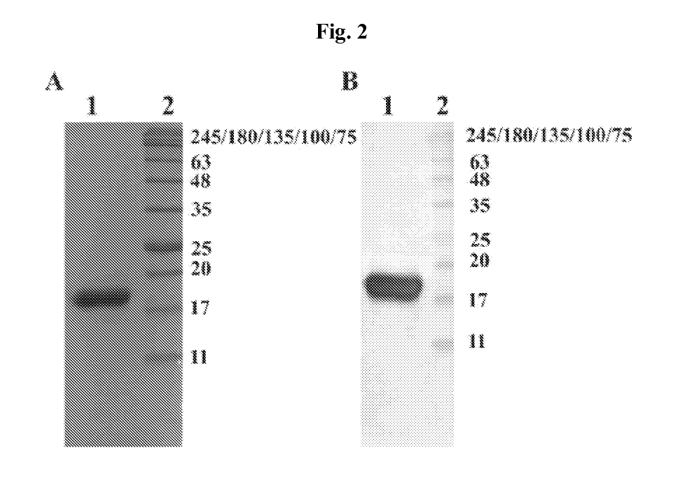

Mature, secreted human MANF is a helical protein with a length of 158 amino acid residues. See Table I (SEQ ID NO:3) and FIG. 2 for MANF's protein sequence and three dimensional structure, respectively. The N-terminal domain (N-domain) of MANF (encompassing residues L20-L120) is entirely helical, with four ca-helices and a rare structural element, a π helix, immediately followed by a 3₁₀ helix.

The C-terminal domain (C-domain) of MANF encompasses residues T126-L158. This domain is also entirely helical and contains one disulfide bond between conserved cysteines in the CXXC motif between α-helices 5 and 6. The CXXC motif is a consensus sequence of proteins of the thiol-protein oxidoreductase superfamily, other members of which include thioredoxins, glutaredoxins, and peroxiredoxins. Common to this enzyme superfamily is that all members are involved in disulfide mediated redox reactions and glutathione metabolism in which the CXXC domain takes center stage. The MANF C-domain is structurally similar to SAP-domains (SAF-A/B, Acinus, PLAS) and most similar to the SAP-domain of Ku70. Ku70 is a cytoplasmic protein with anti-apoptotic activity. Ku70 is associated with Bax, keeping the latter in an inactive conformation. Once Bax dissociates from Ku70 the mitochondrial cell death pathway can be activated.

MANF also refers to any MANF family protein or active fragments thereof. The MANF family protein can be MANF, CDNF, or a fragment thereof. As used herein, MANEF or CNDF peptide comprises a protein having 70, 80, 85, 90, 95, 96, 97, 98, 99, or 100% homology (or identity) with the sequence of human: MANF or CDNF. In some embodiments, fragments of these proteins can include peptides with a length of about 4-40 amino acids; for example, about: 4-40, 4-35, 4-30, 4-25, 4-20, 4-15, 4-10, 5-40, 6-40, 7-40, 8-40, 5-35, 5-30, 5-25, 5-20, 5-15, 5-10, 6-35, 6-30, 6-30, 6-25, 6-20, 6-15, 6-10, 7-35, 7-30, 7-25, 7-20, 7-15, 7-10, 8-35, 8-30, 8-25, 8-20, or 8-15 amino acids. For example, the peptide can consist of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

MANF family proteins can be glycosylated. MANF family proteins can be non-glycosylated.

Either MANE or CDNF can be the pro-form, which contains a signal sequence, or the mature, secreted form in which the signal sequence is cleaved off.

MANF family proteins can be a pro-form of MANF or an active fragment thereof. For example, the peptide sequence of the MANF family protein can comprise or consist of a sequence that has at least about 80% identity with SEQ ID NO: 1. In another example, the peptide sequence of the MANF family protein can comprise or consist of a sequence that has at least about 90% identity with SEQ ID NO: 1. In another example, the peptide sequence of the MANF family protein can comprise or consist of a sequence that has at least about 95% identity with SEQ ID NO: 1. In another example, the peptide sequence of the MANF family protein can comprise or consist of a sequence that has at least about 97% identity with SEQ ID NO: 1. In another example, the peptide sequence of the MANF family protein can comprise or consist of a sequence that has 100% identity with SEQ ID NO: 1. In any of these examples, the MANF family protein can have a length that is at least about 5% the length of SEQ ID NO: 1. In any of these examples, the MANF family protein can have a length that is at least about 50% the length of SEQ ID NO: 1. In any of these examples, the MANF family protein can have a length that is at least about 80% the length of SEQ ID NO: 1. In any of these examples, the MANF family protein can have a length that is at least about 90% the length of SEQ ID NO: 1. In any of these examples, the MANF family protein can have a length that is the same length as SEQ ID NO: 1. The MANF family protein, in any of these examples can also have a maximum length. The maximum length can be, e.g., 100%, 90%, 80%, 70%, 60%, 50%, or 25% the length of SEQ ID NO: 1.

MANF family proteins can be a pro-form of MANF or an active fragment thereof. For example, the peptide sequence of the MANF family protein can comprise or consist of a sequence that has at least about 80% identity with SEQ ID NO: 2. In another example, the peptide sequence of the MANF family protein can comprise or consist of a sequence that has at least about 90% identity with SEQ ID NO: 2. In another example, the peptide sequence of the MANF family protein can comprise or consist of a sequence that has at least about 95% identity with SEQ ID NO: 2. In another example, the peptide sequence of the MANF family protein can comprise or consist of a sequence that has at least about 97% identity with SEQ ID NO: 2. In another example, the peptide sequence of the MANF family protein can comprise or consist of a sequence that has 100% identity with SEQ ID NO: 2. In any of these examples, the MANF family protein can have a length that is at least about 5% the length of SEQ ID NO: 2. In any of these examples, the MANF family protein can have a length that is at least about 50% the length of SEQ ID NO: 2. In any of these examples, the MANF family protein can have a length that is at least about 80% the length of SEQ ID NO: 2. In any of these examples, the MANE family protein can have a length that is at least about 90% the length of SEQ ID NO: 2. In any of these examples, the MANF family protein can have a length that is the same length as SEQ ID NO: 2. The MANF family protein, in any of these examples can also have a maximum length. The maximum length can be, e.g., 100%, 90%, 80%, 70%, 60%, 50%, or 25% the length of SEQ ID NO: 2.

MANF family proteins can be a mature or secreted form of MANE, or an active fragment thereof. For example, the peptide sequence of the MANE family protein can comprise or consist of a sequence that has at least about 80% identity with SEQ ID NO: 3. In another example, the peptide sequence of the MANF family protein can comprise or consist of a sequence that has at least about 90% identity with SEQ ID NO: 3. In another example, the peptide sequence of the MANF family protein can comprise or consist of a sequence that has at least about 95% identity with SEQ ID NO: 3. In another example, the peptide sequence of the MANF family protein can comprise or consist of a sequence that has at least about 97% identity with SEQ ID NO: 3. In another example, the peptide sequence of the MANF family protein can comprise or consist of a sequence that has 100% identity with SEQ ID NO: 3. In any of these examples, the MANF family protein can have a length that is at least about 5% the length of SEQ ID NO: 3. In any of these examples, the MANF family protein can have a length that is at least about 50% the length of SEQ ID NO: 3. In any of these examples, the MANF family protein can have a length that is at least about 80% the length of SEQ ID NO: 3. In any of these examples, the MANEF family protein can have a length that is at least about 90% the length of SEQ ID NO: 3. In any of these examples, the MANF family protein can have a length that is the same length as SEQ ID NO: 3. The MANF family protein, in any of these examples can also have a maximum length. The maximum length can be, e.g., 100%, 90%, 80%, 70%, 60%, 50%, or 25% the length of SEQ ID NO: 3.

MANF family proteins can be a synthetic form of MANF, or an active fragment thereof. The synthetic form of MANF contains a non-natural N-terminal methionine. The N-terminal methionine can enable production of the synthetic form of MANF in cell lines lacking the post-translational modification machinery to process the pro-form of MANF to the secreted or mature form of MANF. For example, the peptide sequence of the MANF family protein can comprise or consist of a sequence that has at least about 80% identity with SEQ ID NO: 4. In another example, the peptide sequence of the MANF family protein can comprise or consist of a sequence that has at least about 90% identity with SEQ ID NO: 4. In another example, the peptide sequence of the MANF family protein can comprise or consist of a sequence that has at least about 95% identity with SEQ ID NO: 4. In another example, the peptide sequence of the MANF family protein can comprise or consist of a sequence that has at least about 97% identity with SEQ ID NO: 4. In another example, the peptide sequence of the MANF family protein can comprise or consist of a sequence that has 100% identity with SEQ ID NO: 4. In any of these examples, the MANF family protein can have a length that is at least about 5% the length of SEQ ID NO: 4. In any of these examples, the MANF family protein can have a length that is at least about 50% the length of SEQ ID NO: 4. In any of these examples, the MANF family protein can have a length that is at least about 80% the length of SEQ ID NO: 4. In any of these examples, the MANF family protein can have a length that is at least about 90% the length of SEQ ID NO: 4. In any of these examples, the MANF family protein can have a length that is the same length as SEQ ID NO: 4. The MANF family protein, in any of these examples can also have a maximum length. The maximum length can be, e.g., 100%, 90%, 80%, 70%, 60%, 50%, or 25% the length of SEQ ID NO: 4.

TABLE 1
Human MANF Protein Sequences

| | | ASCESSION | |
|---|---|---|---|
| SEQ ID | NAME | Number | SEQUENCE |
| SEQ ID | Human Pro- | NP_006001 | MRRMRRMWAT QGLAVALALS VLPGSRALRP GDCEVCISYL |
| NO: 1 | MANF | | GRFYQDLKDR DVTFSPATIE NELIKFCREA RGKENRLCYY |
| | | | IGATDDAATK IINEVSKPLA HHIPVEKICE KLKKKDSQIC |
| | | | ELKYDKQIDL STVDTKKLRV KELKKILDDW GETCKGCAEK |
| | | | SDYIRKINEL MPKYAPKAAS ARTDL |
| SEQ ID | Human Pro- | | MWATQGLAVA LALSVLPGSR ALRPGDCEVC ISYLGRFYQD |
| NO: 2 | MANE | | LKDRDVTFSP ATIENELIKF CREARGKENR LCYYIGATDD |
| | | | AATKIINEVS KPLAHHIPVE KICEKLKKKD SQICELKYDK |
| | | | QIDLSTVDLK KLRVKELKKI LDDWGETCKG CAEKSDYIRK |
| | | | INELMPKYAP KAASARTDL |
| SEQ ID | Human MANF | | LRPGDCEVCI SYLGRFYQDL KDRDVTFSPA TIENELIKFC |
| NO: 3 | (Secreted | | REARGKENRL CYYIGATDDA ATKIINEVSK PLAHHIPVEK |
| | Form) | | ICEKLKKKDS QICELKYDKQ IDLSTVDLKK LRVKELKKIL |
| | | | DDWGETCKGC AEKSDYIRKI NELMPKYAPK AASARTDL |
| SEQ ID | Human | | MLRPGDCEVC ISYLGRFYQD LKDRDVTFSP ATIENELIKF |
| NO: 4 | Synthetic | | CREARGKENR LCYYIGATDD AATKIINEVS KPLAHHIPVE |
| | MANF | | KICEKLKKKD SQICELKYDK QIDLSTVDLK KLRVKELKKI |
| | | | LDDWGETCKG CAEKSDYIRK INELMPKYAP KAASARTDL |

MANF family proteins can be a pro-form of CDNF, or an active fragment thereof. For example, the peptide sequence of the MANF family protein can comprise or consist of a sequence that has at least about 80% identity with SEQ ID NO: 5. In another example, the peptide sequence of the MANF family protein can comprise or consist of a sequence that has at least about 90% identity with SEQ ID NO: 5. In another example, the peptide sequence of the MANF family protein can comprise or consist of a sequence that has at least about 95% identity with SEQ ID NO: 5. In another example, the peptide sequence of the MANF family protein can comprise or consist of a sequence that has at least about 97% identity with SEQ ID NO: 5. In another example, the peptide sequence of the MANF family protein can comprise or consist of a sequence that has 100% identity with SEQ ID NO: 5. In any of these examples, the MANF family protein can have a length that is at least about 5% the length of SEQ ID NO: 5. In any of these examples, the MANF family protein can have a length that is at least about 50% the length of SEQ ID NO: 5. In any of these examples, the MANF family protein can have a length that is at least about 80% the length of SEQ ID NO: 5. In any of these examples, the MANF family protein can have a length that is at least about 90% the length of SEQ ID NO: 5. In any of these examples, the MANF family protein can have a length that is the same length as SEQ ID NO: 5. The MANF family protein, in any of these examples can also have a maximum length. The maximum length can be, e.g., 100%, 90%, 80%, 70%, 60%, 50%, or 25% the length of SEQ ID NO: 5.

MANF family proteins can be a mature or secreted form of CDNF, or an active fragment thereof. For example, the peptide sequence of the MANF family protein can comprise or consist of a sequence that has at least about 80% identity with SEQ ID NO: 6. In another example, the peptide sequence of the MANF family protein can comprise or consist of a sequence that has at least about 90% identity with SEQ ID NO: 6. In another example, the peptide sequence of the MANF family protein can comprise or consist of a sequence that has at least about 95% identity with SEQ ID NO: 6. In another example, the peptide sequence of the MANF family protein can comprise or consist of a sequence that has at least about 97% identity with SEQ ID NO: 6. In another example, the peptide sequence of the MANF family protein can comprise or consist of a sequence that has 100% identity with SEQ ID NO: 6. In any of these examples, the MANF family protein can have a length that is at least about 5% the length of SEQ ID NO: 6. In any of these examples, the MANF family protein can have a length that is at least about 50% the length of SEQ ID NO: 6. In any of these examples, the MANF family protein can have a length that is at least about 80% the length of SEQ ID NO: 6. In any of these examples, the MANF family protein can have a length that is at least about 90% the length of SEQ ID NO: 6. In any of these examples, the MANF family protein can have a length that is the same length as SEQ ID NO: 6. The MANF family protein, in any of these examples can also have a maximum length. The maximum length can be, e.g., 100%, 90%, 80%, 70%, 60%, 50%, or 25% the length of SEQ ID NO: 6.

MANF family proteins can be a synthetic form of CDNF, or an active fragment thereof. The synthetic form of CDNF contains a non-natural N-terminal methionine. The N-terminal methionine can enable production of the synthetic form of CDNF in cell lines lacking the post-translational modification machinery to process the pro-form of CDNF to the secreted or mature form of CDNF. For example, the peptide sequence of the MANF family protein can comprise or consist of a sequence that has at least about 80% identity with SEQ ID NO: 7. In another example, the peptide sequence of the MANF family protein can comprise or consist of a sequence that has at least about 90% identity with SEQ ID NO: 7. In another example, the peptide sequence of the MANF family protein can comprise or consist of a sequence that has at least about 95% identity with SEQ ID NO: 7. In another example, the peptide sequence of the MANF family protein can comprise or consist of a sequence that has at least about 97% identity with SEQ ID NO: 7. In another example, the peptide sequence of the MANE family protein can comprise or consist of a sequence that has 100% identity with SEQ ID NO: 7. In any of these examples, the MANF family protein can have a length that is at least about 5% the length of SEQ ID NO: 7. In any of these examples, the MANF family protein can have a length that is at least about 50% the length of SEQ ID NO: 7. In any of these examples, the MANF family protein can have a length that is at least about 80% the length of SEQ ID NO: 7. In any of these examples, the MANF family protein can have a length that is at least about 90% the length of SEQ ID NO: 7. In any of these examples, the MANF family protein can have a length that is the same length as SEQ ID NO: 7. The MANF family protein, in any of these examples can also have a maximum length. The maximum length can be, e.g., 100%, 90%, 80%, 70%, 60%, 50%, or 25% the length of SEQ ID NO: 7.

TABLE 2
Human CDNF Protein Sequences

| | | ASCESSION | |
|---|---|---|---|
| SEQ ID | NAME | Number | SEQUENCE |
| SEQ ID | Human CDNF | NP_001025125 | MWCASPVAVV AFCAGLLVSH PVLTQGQEAG GRPGADCEVC |
| NO: 5 | Precursor | | KEFLNRFYKS LIDRGVNFSL DTIEKELISF CLDTKGKENR |
| | | | LCYYLGATKD AATKILSEVT RPMSVHMPAM KICEKLKKLD |
| | | | SQICELKYEK TLDLASVDLR KMRVAELKQI LHSWGEECRA |
| | | | CAEKTDYVNL IQELAPKYAA THPKTEL |
| SEQ ID | Human CDNF | | QEAGGRPGAD CEVCKEFLNR FYKSLIDRGV NFSLDTIEKE |
| NO: 6 | (Mature) | | LSIFCLDTKG KENRLCYYLG ATKDAATKIL SEVTRPMSVH |
| | | | MPAMKICEKL KKLDSQICEL KYEKTLDLAS VDLRKMRVAE |
| | | | LKQILHSWGE ECRACAEKTD YVNLIQELAP KYAATHPKTE |
| | | | L |
| SEQ ID | Human | | MQEAGGRPGA DCEVCKEFLN RFYKSLIDRG VNFSLDTIEK |
| NO: 7 | Synthetic | | ELISFCLDTK GKENRLCYYL GATKDAATKI LSEVTRPMSV |
| | CDNF | | HMPAMKICEK LKKLDSQICE LKYEKTLDLA SVDLRKMRVA |
| | | | ELKQILHSWG EECRACAEKT DYVNLIQELA PKYAATHPKT |
| | | | EL |

Active fragments of MANF or CDNF can include short peptides with a length of about 4-40 amino acids; for example, about: 4-40, 4-35, 4-30, 4-25, 4-20, 4-15, 4-10, 5-40, 6-40, 7-40, 8-40, 5-35, 5-30, 5-25, 5-20, 5-15, 5-10, 6-35, 6-30, 6-25, 6-20, 6-15, 6-10, 7-35, 7-30, 7-25, 7-20, 7-15, 7-10, 8-35, 8-30, 8-25, 8-20, or 8-15 amino acids. For example, the preferred peptides can consist of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. The peptides may comprise any of the naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine as well as non-conventional or modified amino acids. The peptide can have 70, 80, 85, 90, 95, 96, 97, 98, 99, or 100% homology (or identity) with the sequence of human CDNF or MANF protein. In some embodiments, the peptides comprise the sequence CXXC. In some embodiments, the peptides comprise the sequence CKGC (SEQ ID NO:94) or CRAC (SEQ ID NO:183) (see, e.g., WO 2013/034805). These peptides can be cell permeable. Active fragments of MANF can include any of the short peptides disclosed in Table 3. Active fragments of CDNF can include any of the short peptides disclosed in Table 4.

TABLE 3
Short peptides of human MANF.

| SEQ ID NO | SEQUENCE |
|---|---|
| SEQ ID NO: 8 | ILDDWGETCKGCAEKSDYIRKINELMPKYAPKAAS |
| | ARTDL |
| SEQ ID NO: 9 | LDDWGETCKGCAEKSDYIRKINELMPKYAPKAASA |
| | RTDL |
| SEQ ID NO: 10 | DDWGETCKGCAEKSDYIRKINELMPKYAPKAASAR |
| | TDL |
| SEQ ID NO: 11 | DWGETCKGCAEKSDYIRKINELMPKYAPKAASART |
| | DL |
| SEQ ID NO: 12 | WGETCKGCAEKSDYIRKINELMPKYAPKAASARTD |
| | L |
| SEQ ID NO: 13 | GETCKGCAEKSDYIRKINELMPKYAPKAASARTDL |
| SEQ ID NO: 14 | ETCKGCAEKSDYIRKINELMPKYAPKAARTDL |
| SEQ ID NO: 15 | TCKGCAEKSDYIRKINELMPKYAPKAASARTDL |
| SEQ ID NO: 16 | CKGCAEKSDYIRKINELMPKYAPKAASARTDL |
| SEQ ID NO: 17 | CKGCAEKSDYIRKIN |
| SEQ ID NO: 18 | TCKGCAEKSDYIRKI |
| SEQ ID DO: 19 | ETCKGCAEKSDYIRK |
| SEQ ID NO: 20 | GETCKGCAEKSDYIR |
| SEQ ID NO: 21 | WGETCKGCAEKSDYI |
| SEQ ID NO: 22 | DWGETCKGCAEKSDY |
| SEQ ID NO: 23 | DDWGETCKGCAEKSD |
| SEQ ID NO: 24 | LDDWGETCKGCAEKS |
| SEQ ID NO: 25 | ILDDWGETCKCAEK |
| SEQ ID NO: 26 | KILDDWGETCKGCAE |
| SEQ ID NO: 27 | KKILDDWGETCKGCA |
| SEQ ID NO: 28 | LKKILDDWGETCKGC |
| SEQ ID NO: 29 | CKGCAEKSDYIRKI |
| SEQ ID NO: 30 | TCKGCAEKSDYIRK |
| SEQ ID NO: 31 | ETCKGCAEKSDYIR |
| SEQ ID NO: 32 | GETCKGCAEKSDYI |
| SEQ ID NO: 33 | WGETCKGCAEKSDY |
| SEQ ID NO: 34 | DWGETCKGCAEKSD |
| SEQ ID NO: 35 | DDWGETCKGCAEKS |
| SEQ ID NO: 36 | LDDWGETCKGCAEK |
| SEQ ID NO: 37 | ILDDWGETCKGCAE |
| SEQ ID NO: 38 | KILDDWGETCKGCA |
| SEQ ID NO: 39 | KKILDDWGETCKGC |
| SEQ ID NO: 40 | CKGCAEKSDYIRK |
| SEQ ID NO: 41 | TCKGCAEKSDYIR |
| SEQ ID NO: 42 | ETCKGCAEKSDYI |
| SEQ ID NO: 43 | GETCKGCAEKSDY |
| SEQ ID NO: 44 | WGETCKGCAEKSD |
| SEQ ID NO: 45 | DWGETCKGCAEKS |
| SEQ ID NO: 46 | DDWGETCKGCAEK |
| SEQ ID NO: 47 | LDDWGETCKGCAE |
| SEQ ID NO: 48 | ILDDWGETCKGCA |
| SEQ ID NO: 49 | KILDDWGETCKGC |
| SEQ ID NO: 50 | CKGCAEKSDYIR |
| SEQ ID NO: 51 | TCKGCAEKSDYI |
| SEQ ID NO: 52 | ETCKGCAEKSDY |
| SEQ ID NO: 53 | GETCKGCAEKSD |
| SEQ ID NO: 54 | WGETCKGCAEKS |
| SEQ ID NO: 55 | DWGETCKGCAEK |
| SEQ ID NO: 56 | DDWGETCKGCAE |
| SEQ ID NO: 57 | LDDWGETCKGCA |
| SEQ ID NO: 58 | ILDDWGETCKGC |
| SEQ ID NO: 59 | CKGCAEKSDYI |
| SEQ ID NO: 60 | TCKGCAEKSDY |
| SEQ ID NO: 61 | ETCKGCAEKSD |
| SEC ID NO: 62 | GETCKGCAEKS |
| SEQ ID NO: 63 | WGETCKGCAEK |
| SEQ ID NO: 64 | DWGETCKGCAE |
| SEQ ID NO: 65 | DDWGETCKGCA |
| SEQ ID NO: 66 | LDDWGETCKGC |
| SEQ ID NO: 67 | CKGCAEKSDY |
| SEQ ID NO: 68 | TCKGCAEKSD |
| SEC ID NO: 69 | ETCKGCAEKS |
| SEQ ID NO: 70 | GETCKGCAEK |
| SEQ ID NO: 71 | WGETCKGCAE |
| SEQ ID NO: 72 | DWGETCKGCA |
| SEQ ID NO: 73 | DDWGETCKGC |
| SEQ ID NO: 74 | CKGCAEKSD |
| SEQ TD NO: 75 | TCKGCAEKS |
| SEC ID NO: 76 | ETCKGCAEK |
| SEQ ID NO: 77 | GETCKGCAE |
| SEQ ID NO: 78 | WGETCKGCA |
| SEQ ID NO: 79 | DWGETCKGC |
| SEQ ID NO: 80 | CKGCAEKS |
| SEQ ID NO: 81 | TCKGCAEK |
| SEQ ID NO: 82 | ETCKGCAE |
| SEC ID NO: 83 | GETCKGCA |
| SEQ ID NO: 84 | WGETCKGC |
| SEQ ID NO: 85 | CKGCAEK |
| SEQ ID NO: 86 | TCKGCNE |
| SEQ ID NO: 87 | ETCKGCA |
| SEQ ID NO: 88 | GETCKGC |
| SEQ ID NO: 89 | CKGCAE |
| SEQ ID NO: 90 | TCKGCA |
| SEQ ID NO: 91 | ETCKGC |
| SEQ ID NO: 92 | CKGCA |
| SEQ ID NO: 93 | TCKGC |
| SEQ ID NO: 94 | CKGC |

TABLE 4
Short peptides of human CDNF.

| SEQ ID NO | SEQUENCE |
|---|---|
| SEQ ID NO: 95 | KQILHSWGEECRACAEKTDYVNLIQELAPKYAA |
| | THPKTEL |
| SEQ ID NO: 96 | QILHSWGEECRACAEKTDYVNLIQELAPKYAAT |
| | HPKTEL |
| SEQ ID NO: 97 | ILHSWGEECRACAEKTDYVNLIQELAPKYAATH |
| | PKTEL |
| SEQ ID NO: 98 | LHSWGEECRACAEKTDYVNLIQELAPKYAATHP |
| | KTEL |
| SEQ ID NO: 99 | HSWGEECRACAEKTDYVNLIQELAPKYAATHPK |
| | TEL |
| SEQ ID NO: 100 | SWGEECRACAEKTDYVNLIQELAPKYAATHPKT |
| | EL |
| SEQ ID NO: 101 | WGEECRACAEKTDYVNLIQELAPKYAATHPKTE |
| | L |
| SEQ ID NO: 102 | GEECRACAEKTDYVNLIQELAPKYAATHPKTEL |
| SEQ ID NO: 103 | EECRACAEKTDYVNLIQELAPKYAATHPKTEL |
| SEQ ID NO: 104 | ECRACAEKTDYVNLIQELAPKYAATHPKTEL |
| SEQ ID NO: 105 | CRACAEKTDYVNLIQELAPKYAATHPKTEL |
| SEQ ID NO: 106 | LKQILHSWGEECRAC |
| SEQ ID NO: 107 | KQILHSWGEECRACA |
| SEQ ID NO: 108 | QILHSWGEECRACAE |
| SEQ ID NO: 109 | ILHSWGEECRACAEK |
| SEQ ID NO: 110 | LHSWGEECRACAEKT |
| SEQ ID NO: 111 | HSWGEECRACAEKTD |
| SEQ ID NO: 112 | SWGEECRACAEKTDY |
| SEQ ID NO: 113 | WGEECRACAEKTDYV |
| SEQ ID NO: 114 | GEECRACAEKTDYVN |
| SEQ ID NO: 115 | EECRACAEKTDYVNL |
| SEQ ID NO: 116 | ECRACAEKTDYVNLI |
| SEQ ID NO: 117 | CRACAEKTDYVNLIQ |
| SEQ ID NO: 118 | KQILHSWGEECRAC |
| SEQ ID NO: 119 | QILHSWGEECRACA |
| SEQ ID NO: 120 | ILHSWGEECRACAE |
| SEQ ID NO: 121 | LHSWGEECRACEK |
| SEQ ID NO: 122 | HSWGEECRACAEKT |
| SEQ ID NO: 123 | SWGEECRACAEKTD |
| SEQ ID NO: 124 | WGEECRACAEKTDY |
| SEQ ID NO: 125 | GEECRACAEKTDYV |
| SEQ ID NO: 126 | EECRACAEKTDYVN |
| SEQ ID NO: 127 | ECRACAEKTDYVNL |
| SEQ ID NO: 128 | CRACAEKTDYVNLI |
| SEQ ID NO: 129 | QILHSWGEECRAC |
| SEQ ID NO: 130 | ILHSWGEECRACA |
| SEQ ID NO: 131 | LHSWGEECRACAE |
| SEQ ID NO: 132 | HSWGEECRACAEK |
| SEQ ID NO: 133 | SWGEECRACAEKT |
| SEQ ID NO: 134 | WGEECRACAEKTD |
| SEQ ID NO: 135 | GEECRACAEKTDY |
| SEQ ID NO: 136 | EECRACAEKTDYV |
| SEQ ID NO: 137 | ECRACAEKTDYVN |
| SEQ ID NO: 138 | CRACAEKTDYVNL |
| SEQ ID NO: 139 | ILHSWGEECRAC |
| SEQ ID NO: 140 | LHSWGEECRACA |
| SEQ ID NO: 141 | HSWGEECRACAE |
| SEQ ID NO: 142 | SWGEECRACAEK |
| SEQ ID NO: 143 | WGEECRACAEKT |
| SEQ ID NO: 144 | GEECRACAEKTD |
| SEQ ID NO: 145 | EECRACAEKTDY |
| SEQ ID NO: 146 | ECRACAEKTDYV |
| SEQ ID NO: 147 | CRACAEKTDYVN |
| SEQ ID NO: 148 | LHSWGEECRAC |
| SEQ ID NO: 149 | HSWGEECRACA |
| SEQ ID NO: 150 | SWGEECRACAE |
| SEQ ID NO: 151 | WGEECRACAEK |
| SEQ ID NO: 152 | GEECRACAEKT |
| SEQ ID NO: 153 | EECRACAEKTD |
| SEQ ID NO: 154 | ECRACAEKTDY |
| SEQ ID NO: 155 | CRACAEKTDYV |
| SEQ ID NO: 156 | HSWGEECRAC |
| SEQ ID NO: 157 | SWGEECRACA |
| SEQ ID NO: 158 | WGEECRACAE |
| SEQ ID NO: 159 | GEECRACAEK |
| SEQ ID NO: 160 | EECRACAEKT |
| SEQ ID NO: 161 | ECRACAEKTD |
| SEQ ID NO: 162 | CRACAEKTDY |
| SEQ ID NO: 163 | SWGEECRAC |
| SEQ ID NO: 164 | WGEECRACA |
| SEQ ID NO: 165 | GEECRACAE |
| SEQ ID NO: 166 | EECRACAEK |
| SEQ ID NO: 167 | ECRACAEKT |
| SEQ ID NO: 168 | CRACAEKTD |
| SEQ ID NO: 169 | WGEECRAC |
| SEQ ID NO: 170 | GEECRACA |
| SEQ ID NO: 171 | EECRACAE |
| SEQ ID NO: 172 | ECRACAEK |
| SEQ ID NO: 173 | CRACAEKT |
| SEQ ID NO: 174 | GEECRAC |
| SEQ ID NO: 175 | EECRACA |
| SEQ ID NO: 176 | ECRACAE |
| SEQ ID NO: 177 | CRACAEK |
| SEQ ID NO: 178 | EECRAC |
| SEQ ID NO: 179 | ECRACA |
| SEQ ID NO: 180 | CRACAE |
| SEQ ID NO: 181 | ECRAC |
| SEQ ID NO: 182 | CRACA |
| SEQ ID NO: 183 | CRAC |

The peptides can be conjugated to a detectable chemical or biochemical moiety such as a FITC-label. As used herein, a “detectable chemical or biochemical moiety” means a tag that exhibits an amino acid sequence or a detectable chemical or biochemical moiety for the purpose of facilitating detection of the peptide; such as a detectable molecule selected from among: a visible, fluorescent, chemiluminescent, or other detectable dye; an enzyme that is detectable in the presence of a substrate, e.g., an alkaline phosphatase with NBT plus BCIP or a peroxidase with a suitable substrate; a detectable protein, e.g., a green fluorescent protein. Preferably, the tag does not prevent or hinder the penetration of the peptide into the target cell.

Pharmaceutical Compositions

The active ingredients can be provided in a pharmaceutical composition. The pharmaceutical composition can comprise pharmaceutically acceptable diluent(s), excipient(s), or carrier(s). The pharmaceutical compositions can include other medicinal or pharmaceutical agents, carriers, adjuvants, such as preserving, stabilizing, wetting or emulsifying agents, solution promoters, salts for regulating the osmotic pressure, and/or buffers. Methods well known in the art for making formulations are to be found in, for example. Remington: The Science and Practice of Pharmacy, (20th ed.) ed. A. R. Gennaro A R., 2000, Lippencott Williams & Wilkins.

It will be evident to those skilled in the art that the number and frequency of administration will be dependent upon the response of the host. “Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

The pharmaceutical compositions may be in any form that allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal (e.g., as a spray) and also subcutaneous injections, intravenous, intramuscular, intrasternal, intracavernous, intrathecal, intrameatal, intraurethral injection or infusion techniques.

Pharmaceutical compositions comprising a MANF family protein can be formulated for delivery to the eye. For example, the pharmaceutical compositions can be formulated for topical administration, intravitreal administration, intracameral administration, subconjunctival administration, subtenon administration, retrobulbar administration, posterior juxtascleral administration, or a combination thereof. In some embodiments, the pharmaceutical compositions comprising the MANF family protein are formulated for topical administration. In some embodiments, the pharmaceutical compositions comprising the MANF family protein are formulated for intravitreal administration.

The pharmaceutical compositions and formulations disclosed herein can comprise one or more pharmaceutically acceptable excipients. The pharmaceutically acceptable excipients can comprise acacia, acesulfame potassium, acetic acid glacial, acetone, acetyltributyl citrate, acetyltriethyl citrate, adipic acid, agar, albumnin, alcohol, alginic acid, aliphatic polyesters, alitame, almond oil, alpha tocopherol, aluminum hydroxide adjuvant, aluminum monostearate, aluminum oxide, aluminum phosphate adjuvant, ammonia solution, ammonium alginate, ammonium chloride, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylene glycol, butylparabel, calcium acetate, calcium alginate, calcium carbonate, calcium chloride, calcium hydroxide, calcium lactate, calcium phosphate dibasic anhydrous, calcium phosphate dibasic dihydrate, calcium phosphate tribasic, calcium silicate, calcium stearate, calcium sulfate, canola oil, carbomer, carbon dioxide, carboxymethylcellulose calcium, carboxymethylcellulose sodium, carrageenan, castor oil, castor oil hydrogenated, cellulose microcrystalline, cellulose microcrystalline and carboxymethylcellulose sodium, cellulose powdered, cellulose silicified microcrystalline, cellulose acetate, cellulose acetate phthalate, ceratonia, ceresin, cetostearyl alcohol, cetrimide, cetyl alcohol, cetylpyridinium chloride, chitosan, chlorhexidine, chlorobutanol, chlorocresol, chlorodifluoroethane (hcfc), chlorofluorocarbons (cfc), chloroxylenol, cholesterol, citric acid monohydrate, coconut oil, colloidal silicon dioxide, coloring agents, copovidone, corn oil, corn starch and pregelatinized starch, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane (hfc), dimethicone, dimethyl ether, dimethyl phthalate, dimethyl sulfoxide, dimethylacetamiide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol stearates, ethylene vinyl acetate, ethylparaben, fructose, fumaric acid, gelatin, glucose liquid, glycerin, glyceryl behenate, glyceryl monooleate, glyceryl monostearate, glyceryl paimitostearate, glycine, glycofurol, guar gum, hectorite, heptafluoropropane (hfc), hexetidine, hydrocarbons (hc), hydrochloric acid, hydrophobic colloidal silica, hydroxyethyl cellulose, hydroxyethylmethyl cellulose, hydroxypropyl betadex, hydroxypropyl cellulose, hydroxypropyl cellulose low substituted, hydroxypropyl starch, hypromellose, hypromellose acetate succinate, hypromellose phthalate, imidurea, inulin, iron oxides, isomalt, isopropyl alcohol, isopropyl myristate, isopropyl palmitate, kaolin, lactic acid, lactitol, lactose anhydrous, lactose inhalation, lactose monohydrate, lactose monohydrate and corn starch, lactose monohydrate and microcrystalline cellulose, lactose monohydrate and povidone, lactose monohydrate and powdered cellulose, lactose spray dried, lanolin, lanolin hydrous, lanolin alcohols, lauric acid, lecithin, leucine, linoleic acid, macrogol 15 hydroxystearate, magnesium aluminum silicate, magnesium carbonate, magnesium oxide, magnesium silicate, magnesium stearate, magnesium trisilicate, maleic acid, malic acid, maititol, maltitol solution, maltodextrin, maltol, maltose, mannitol, medium chain triglycerides, meglumine, menthol, methionine, methylcellulose, methylparaben, mineral oil, mineral oil light, mineral oil and lanolin alcohols, monoethanolamine, monosodium glutamate, monothioglycerol, myristic acid, myristyl alcohol, neohesperidin dihydrochalcone, neotame, nitrogen, nitrous oxide, octyldodecanol, oleic acid, oleyl alcohol, olive oil, palmitic acid, paraffin, peanut oil, pectin, pentetic acid, petrolatum, petrolatum and lanolin alcohols, phenol, phenoxyethanol, phenylethyl alcohol, phenylhnercuric acetate, phenyhnercuric borate, phenylmercuric nitrate, phospholipids, phosphoric acid, polacrilin potassium, poloxamer, polycarbophil, polydextrose, poly (dl lactic acid), polyethylene glycol, polyethylene oxide, polymethacrylates, poly(methyl vinylether/maleic anhydride), poiyoxyethylene alkyl ethers, polyoxyethylene castor oil derivatives, polyoxyethylene sorbitan fatty acid esters, polyoxyethylene stearates, polyoxylglycerides, polyvinyl acetate phthalate, polyvinyl alcohol, potassium alginate, potassium alumn, potassiumn benzoate, potassium bicarbonate, potassiumn chloride, potassium citrate, potassium hydroxide, potassium metabisufite, potassium sorbate, povidone, propionic acid, propyl gallate, propylene carbonate, propylene glycol, propylene glycol alginate, propylparaben, propylparaben sodium, pyrrolidone, raffinose, saccharin, saccharin sodium, safflower oil, saponite, sesame oil, shellac, simethicone, sodium acetate, sodium alginate, sodium ascorbate, sodium benzoate, sodium bicarbonate, sodium borate, sodium carbonate, sodium chloride, sodium citrate dihydrate, sodium cyclamate, sodium formaldehyde sulfoxylate, sodium hyaluronate, sodium hydroxide, sodium lactate, sodium lauryl sulfate, sodium metabisulfite, sodium phosphate dibasic, sodium phosphate monobasic, sodium propionate, sodium starch glycolate, sodium stearyl fumarate, sodium sulfite, sodium thiosulfate, sorbic acid, sorbitan esters (sorbitan fatty acid esters), sorbitol, soybean oil, starch, starch pregelatinized, starch sterilizable maize, stearic acid, stearyl alcohol, sucralose, sucrose, sucrose octaacetate, sugar compressible, sugar confectioner's, sugar spheres, sulfobutylether b cyclodextrin, sulfur dioxide, sulfuric acid, sunflower oil, suppository bases hard fat, tagatose, talc, tartaric acid, tetrafluoroethane (hfc), thaumatin, thimerosal, thymol, titanium dioxide, tragacanth, trehalose, triacetin, tributyl citrate, tricaprylin, triethanolamnine, triethyl citrate, triolein, vanillin, vegetable oil hydrogenated, vitamin e polyethylene glycol succinate, water, wax anionic emulsifying, wax carnauba, wax cetyl esters, wax microcrystalline, wax nonionic emulsifying, wax white, wax—yellow, xanthan gum, xylitol, zein, zinc acetate, zinc stearate, or any combination thereof.

### SPECIFIC EMBODIMENTS

For the purposes of clarity and a concise description, specific embodiments are provided below. These specific embodiments are meant to supplement, not replace, the preceding description. Further, the recitation of specific embodiments and definitions below does not exclude the combination of any of the embodiments below with the embodiments and description set forth above.

### Embodiment 1

A method of increasing retinal tolerance time, reducing cell death during an ischemic event in the retina, reducing cell death following an ischemic event in the retina, treating an ischemic event in the retina, or a combination thereof, the method comprising: (a) administering a dose of a pharmaceutical composition comprising an effective amount of a MANF family protein to a subject in need thereof; (b) performing a treatment to resolve a blockage causing the ischemic event.

### Embodiment 2

The method of embodiment 1, wherein the MANF family protein is a mesencephalic astrocyte derived neurotrophic factor (MANF) protein, or a fragment thereof.

### Embodiment 3

The method of embodiment 1, wherein the MANF family protein comprises a sequence that has at least about 80% identity with SEQ ID NO:3.

### Embodiment 4

The method of embodiment 1, wherein the MANF family protein comprises a sequence that has at least about 85% identity with SEQ ID NO:3.

### Embodiment 5

The method of embodiment 1, wherein the MANF family protein comprises a sequence that has at least about 90% identity with SEQ ID NO:3.

### Embodiment 6

The method of embodiment 1, wherein the MANF family protein comprises a sequence that has at least about 95% identity with SEQ ID NO:3.

### Embodiment 7

The method of embodiment 1, wherein the MANF family protein comprises a sequence that has 100% identity with SEQ ID NO:3.

### Embodiment 8

The method of embodiment 1, wherein the MANF family protein consists of a sequence that has at least about 80% identity with SEQ ID NO:3.

### Embodiment 9

The method of embodiment 1, wherein the MANF family protein consists of a sequence that has at least about 85% identity with SEQ ID NO:3.

### Embodiment 10

The method of embodiment 1, wherein the MANF family protein consists of a sequence that has at least about 90% identity with SEQ ID NO:3.

### Embodiment 11

The method of embodiment 1, wherein the MANF family protein consists of a sequence that has at least about 95% identity with SEQ ID NO:3.

### Embodiment 12

The method of embodiment 1, wherein the MANF family protein consists of a sequence that has 100% identity with SEQ ID NO:3.

### Embodiment 13

The method of any one of embodiments 3-12, wherein the MANF family protein has a length that is at least 80% the length of SEQ ID NO:3.

### Embodiment 14

The method of any one of embodiments 3-12, wherein the MANF family protein has a length that is 100% the length of SEQ ID NO:3.

### Embodiment 15

The method of embodiment 1, wherein the MANF family protein consists of a sequence listed in Table 3.

### Embodiment 16

The method of embodiment 15, wherein the MANF family protein is cell permeable.

### Embodiment 17

The method of embodiment 1, wherein the MANF family protein is a conserved dopamine neurotrophic factor (CDNF) protein, or a fragment thereof.

### Embodiment 18

The method of embodiment 1, wherein the MANF family protein comprises a sequence that has at least about 80% identity with SEQ ID NO:6.

### Embodiment 19

The method of embodiment 1, wherein the MANF family protein comprises a sequence that has at least about 85% identity with SEQ ID NO:6.

### Embodiment 20

The method of embodiment 1, wherein the MANF family protein comprises a sequence that has at least about 90% identity with SEQ ID NO:6.

### Embodiment 21

The method of embodiment 1, wherein the MANF family protein comprises a sequence that has at least about 95% identity with SEQ ID NO:6.

### Embodiment 22

The method of embodiment 1, wherein the ML NF family protein comprises a sequence that has 100% identity with SEQ ID NO:6.

### Embodiment 23

The method of embodiment 1, wherein the MANF family protein consists of a sequence that has at least about 80% identity with SEQ ID NO:6.

### Embodiment 24

The method of embodiment 1, wherein the MANF family protein consists of a sequence that has at least about 85% identity with SEQ ID NO:6.

### Embodiment 25

The method of embodiment 1, wherein the MANE family protein consists of a sequence that has at least about 90% identity with SEQ ID NO:6.

### Embodiment 26

The method of embodiment 1, wherein the MANF family protein consists of a sequence that has at least about 95% identity with SEQ ID NO:6.

### Embodiment 27

The method of embodiment 1, wherein the MANF family protein consists of a sequence that has 100% identity with SEQ ID NO:6.

### Embodiment 28

The method of any one of embodiments 18-27, wherein the MANF family protein has a length that is at least 80% the length of SEQ ID NO:3.

### Embodiment 29

The method of any one of embodiments 18-27, wherein the MANF family protein has a length that is 100% the length of SEQ ID NO:3.

### Embodiment 30

The method of embodiment 1, wherein the MANF family protein consists of a sequence listed in Table 4.

### Embodiment 31

The method of embodiment 30, wherein the MANF family protein is cell permeable.

### Embodiment 32

The method of any one of embodiments 1-31, wherein the pharmaceutical composition is administered to an eye of the subject.

### Embodiment 33

The method of embodiment 32, wherein the pharmaceutical composition is administered by topical administration, intravitreal injection, intracameral administration, subconjunctival administration, subtenon administration, retrobulbar administration, posterior juxtascleral administration, or a combination thereof.

### Embodiment 34

The method of embodiment 32, wherein the pharmaceutical composition is administered by topical administration.

### Embodiment 35

The method of embodiment 32, wherein the pharmaceutical composition is administered by intravitreal injection.

### Embodiment 36

The method of any one of embodiments 1-35, wherein the dose is administered after the treatment to resolve the blockage.

### Embodiment 37

The method of any one of embodiments 1-35, wherein the dose is administered prior to the treatment to resolve the blockage.

### Embodiment 38

The method of any one of embodiments 1-37, wherein the dose has a volume of about: 1-500 μL, 10-250 μL, 25-150 μL, 50-100 μL, 1 μL, 5 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 105 μL, 110 μL, 115 μL, 120 μL, 125 μL, 150 μL, 175 μL, 200 μL, 225 μL, 250 μL, 300 μL, 350 μL, 400 μL, 450 μL, or 500 μL.

### Embodiment 39

The method of any one of embodiments 1-37, wherein the dose has a volume of from about 1 μL to about 500 μL.

### Embodiment 40

The method of any one of embodiments 1-37, wherein the dose has a volume of from about 10 μL to about 250 μL.

### Embodiment 41

The method of any one of embodiments 1-37, wherein the dose has a volume of about 25 μL to about 150 μL.

### Embodiment 42

The method of any one of embodiments 1-37, wherein the dose has a volume of from about 50 μL to about 100 μL.

### Embodiment 43

The method of any one of embodiments 1-37, wherein the dose has a volume of from about 25 μL to about 125 μL.

### Embodiment 44

The method of any one of embodiments 1-37, wherein the dose has a volume of about 50 μL.

### Embodiment 45

The method of any one of embodiments 1-37, wherein the dose has a volume of about 100 μL.

### Embodiment 46

The method of any one of embodiments 1-45, wherein the dose has a concentration of the MANF family protein that is about: 0.1-100 mg/mL, 0.1-40 mg/mL, 1-20 mg/mL, 1.5-15 mg/mL, 8.1-32.4 mg/mL, 2.7-5.4 mg/mL, 0.1 mg/mL, 0.5 mg/mL, 1 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 ng/mL, 2 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3 mg/mL, 3.1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mL, 3.9 mg/mL, 4 mg/mL, 4.1 mg/mL, 4.2 mg/mL, 4.3 mg/mL, 4.4 mg/mL, 4.5 mg/mL, 4.6 mg/mL, 4.7 mg/mL, 4.8 mg/mL, 4.9 mg/mL, 5 mg/mL, 5.1 mg/mL, 5.2 mg/mL, 5.3 mg/mL, 5.4 mg/mL, 5.5 mg/mL, 5.6 mg/mL, 5.7 mg/mL, 5.8 mg/mL, 5.9 mg/mL, 6 mg/mL, 6.25 mg/mL, 6.5 mg/mL, 6.75 mg/mL, 7 mg/mL, 7.25 mg/mL, 7.5 mg/mL, 7.75 mg/mL, 8 mg/mL, 8.25 mg/mL, 8.5 mg/mL, 8.75 mg/mL, 9 mg/mL, 9.25 mg/mL, 9.5 mg/mL, 9.75 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 25 mg/mL, 30 mg/mL, 35 mg/mL, 40 mg/mL, 50 mg/mL, 60 mg/mL, 70 mg/mL, 80 mg/mL, 90 mg/mL, or 100 mg/mL.

### Embodiment 47

The method of any one of embodiments 1-45, wherein the dose has a concentration of the MANF family protein that is from about 1 mg/mL to about 20 mg/mL.

### Embodiment 48

The method of any one of embodiments 1-45, wherein the dose has a concentration of the MANF family protein that is from about 1.5 mg/mL to about 15 mg/mL.

### Embodiment 49

The method of any one of embodiments 1-45, wherein the dose has a concentration of the MANF family protein that is from about 2.7 mg/mL to about 5.4 mg/mL.

### Embodiment 50

The method of any one of embodiments 1-49, wherein the effective amount of the MANF family protein is about: 1-5000 μg, 5-2500 μg, 10-2000 μg, 500-2000 mg/mL, 50-1000 μg, 100-500 μg, 200-350 μg, 250-300 μg, 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 525 μg, 550 μg, 575 μg, 600 μg, 625 μg, 650 μg, 675 μg, 700 μg, 750 μg, 800 μg, 850 μg, 900 μg, 950 μg, 1000 μg, 1100 μg, 1200 μg, 1300 μg, 1400 μg, 1500 μg, 1750 μg, 2000 μg, 2500 μg, 3000 μg, 3500 μg, 4000 μg or 5000 μg.

### Embodiment 51

The method of any one of embodiments 1-49, wherein the effective amount of the MANF family protein is from about 50 μg to about 1000 μg.

### Embodiment 52

The method of any one of embodiments 1-49, wherein the effective amount of the MANF family protein is from about 100 μg to about 500 μg.

### Embodiment 53

The method of any one of embodiments 1-49, wherein the effective amount of the MANF family protein is from about 250 μg to about 300 μg.

### Embodiment 54

The method of any one of embodiments 1-53, wherein the dose is administered once every 2 to 8 weeks.

### Embodiment 55

The method of any one of embodiments 1-53, wherein the dose is administered once every 3 to 6 weeks.

### Embodiment 56

The method of any one of embodiments 1-53, wherein the dose is administered once every month.

### Embodiment 57

The method of any one of embodiments 1-53, wherein the dose is administered once every two months.

### Embodiment 58

The method of any one of embodiments 1-53, wherein the dose is administered once every hour.

### Embodiment 59

The method of any one of embodiments 1-53, wherein the dose is administered once every two hours.

### Embodiment 60

The method of any one of embodiments 1-53, wherein the dose is administered once every four hours.

### Embodiment 61

The method of any one of embodiments 1-53, wherein the dose is administered daily.

### Embodiment 62

The method of any one of embodiments 1-53, wherein the dose is only administered once.

### Embodiment 63

The method of any one of embodiments 1-62, wherein the ischemic event is a retinal artery occlusion.

### Embodiment 64

The method of any one of embodiments 1-62, wherein the ischemic event is an acute retinal artery occlusion.

### Embodiment 65

The method of any one of embodiments 1-62, wherein the ischemic event is a central retinal artery occlusion.

### Embodiment 66

The method of any one of embodiments 1-62, wherein the ischemic event is a branch retinal artery occlusion.

### Embodiment 67

The method of any one of embodiments 1-62, wherein the ischemic event is not a chronic retinal artery occlusion.

### Embodiment 68

The method of any one of embodiments 1-62, wherein the ischemic event is a retinal vein occlusion.

### Embodiment 69

The method of any one of embodiments 1-62, wherein the ischemic event is not a retinal vein occlusion.

### Embodiment 70

The method of any one of embodiments 1-69, wherein the treatment to resolve the blockage comprises administration of a vasodilator.

### Embodiment 71

The method of embodiment 70, wherein the vasodilator comprises pentoxyphyline, inhalation of carbogen, hyperbaric oxygen, sublingual isosorbide dinitrite, or a combination thereof.

### Embodiment 72

The method of any one of embodiments 1-71, wherein the treatment to resolve the blockage comprises ocular massage, intravenous acetazolamide, intravenous mannitol, topical antiglaucoma drops, anterior chamber paracentisis, or a combination thereof.

### Embodiment 73

The method of any one of embodiments 1-72, wherein the treatment to resolve the blockage comprises intravenous methylprednisolone.

### Embodiment 74

The method of any one of embodiments 1-73, wherein the treatment to resolve the blockage comprises Nd YAG laser treatment, pars plana vitrectomy, or a combination thereof.

### Embodiment 75

The method of any one of embodiments 1-74, wherein the treatment to resolve the blockage comprises intravenous tissue plasminogen activator, intra-arterial tissue plasminogen activator, or a combination thereof.

### Embodiment 76

The method of any one of embodiments 1-75, wherein the treatment to resolve the blockage comprises panretinal photocoagulation.

### Embodiment 77

The method of any one of embodiments 1-76, wherein the treatment to resolve the blockage comprises administration of a steroid.

### Embodiment 78

The method of any one of embodiments 1-77, wherein the MANE family protein and the treatment to resolve the blockage have a synergistic effect on retinal ganglion cell survival.

### Embodiment 79

The method of any one of embodiments 1-77, wherein the MANF family protein and the treatment to resolve the blockage exhibit therapeutic synergy.

### Embodiment 80

The method of any one of embodiments 1-79, further comprising diagnosing the ischemic event.

### Embodiment 81

The method of embodiment 80, wherein diagnosing is based upon sudden loss of vision in one eye.

### Embodiment 82

The method of embodiment 80 or 81, wherein diagnosing is based upon a determination of retinal opacity, a cherry red spot in the foveal center, the presence of box carring of the blood columns in retinal vessels, absence of arterial circulation based upon flurescein fundus angiography, or a combination thereof.

### Embodiment 83

A method of increasing retinal tolerance time, reducing cell death during a retinal artery occlusion, reducing cell death following a retinal artery occlusion, treating a retinal artery occlusion, or a combination thereof, the method comprising administering a dose of a pharmaceutical composition comprising an effective amount of a MANF family protein to a subject exhibiting one or more symptoms of a retinal artery occlusion.

### Embodiment 84

The method of embodiment 83, wherein the MANF family protein is a mesencephalic astrocyte derived neurotrophic factor (MANEF) protein, or a fragment thereof.

### Embodiment 85

The method of embodiment 83, wherein the MANF family protein comprises a sequence that has at least about 80% identity with SEQ ID NO:3.

### Embodiment 86

The method of embodiment 83, wherein the MANF family protein comprises a sequence that has at least about 85% identity with SEQ ID NO:3.

### Embodiment 87

The method of embodiment 83, wherein the MANF family protein comprises a sequence that has at least about 90% identity with SEQ ID NO:3.

### Embodiment 88

The method of embodiment 83, wherein the MANF family protein comprises a sequence that has at least about 95% identity with SEQ ID NO:3.

### Embodiment 89

The method of embodiment 83, wherein the MANF family protein comprises a sequence that has 100% identity with SEQ ID NO:3.

### Embodiment 90

The method of embodiment 83, wherein the MANF family protein consists of a sequence that has at least about 80% identity with SEQ ID NO:3.

### Embodiment 91

The method of embodiment 83, wherein the MANF family protein consists of a sequence that has at least about 85% identity with SEQ ID NO:3.

### Embodiment 92

The method of embodiment 83, wherein the MANF family protein consists of a sequence that has at least about 90% identity with SEQ ID NO:3.

### Embodiment 93

The method of embodiment 83, wherein the MANF family protein consists of a sequence that has at least about 95% identity with SEQ ID NO:3.

### Embodiment 94

The method of embodiment 83, wherein the MANF family protein consists of a sequence that has 100% identity with SEQ ID NO:3.

### Embodiment 95

The method of any one of embodiments 85-94, wherein the MANF family protein has a length that is at least 80% the length of SEQ ID NO:3.

### Embodiment 96

The method of any one of embodiments 85-94, wherein the MANF family protein has a length that is 100% the length of SEQ ID NO:3.

### Embodiment 97

The method of embodiment 83, wherein the MANE family protein consists of a sequence listed in Table 3.

### Embodiment 98

The method of embodiment 97, wherein the MANF family protein is cell permeable.

### Embodiment 99

The method of embodiment 83, wherein the MANF family protein is a conserved dopamine neurotrophic factor (CDNF) protein, or a fragment thereof.

### Embodiment 100

The method of embodiment 83, wherein the MANF family protein comprises a sequence that has at least about 80% identity with SEQ ID NO:6.

### Embodiment 101

The method of embodiment 83, wherein the MANF family protein comprises a sequence that has at least about 85% identity with SEQ ID NO:6.

### Embodiment 102

The method of embodiment 83, wherein the MAN family protein comprises a sequence that has at least about 90% identity with SEQ ID NO:6.

### Embodiment 103

The method of embodiment 83, wherein the MANF family protein comprises a sequence that has at least about 95% identity with SEQ ID NO:6.

### Embodiment 104

The method of embodiment 83, wherein the MANF family protein comprises a sequence that has 100% identity with SEQ ID NO:6.

### Embodiment 105

The method of embodiment 83, wherein the MANF family protein consists of a sequence that has at least about 80% identity with SEQ ID NO:6.

### Embodiment 106

The method of embodiment 83, wherein the MANF family protein consists of a sequence that has at least about 85% identity with SEQ ID NO:6.

### Embodiment 107

The method of embodiment 83, wherein the MANF family protein consists of a sequence that has at least about 90% identity with SEQ ID NO:6.

### Embodiment 108

The method of embodiment 83, wherein the MANF family protein consists of a sequence that has at least about 95% identity with SEQ ID NO:6.

### Embodiment 109

The method of embodiment 83, wherein the MANF family protein consists of a sequence that has 100% identity with SEQ ID NO:6.

### Embodiment 110

The method of any one of embodiments 101-109, wherein the MANF family protein has a length that is at least 80% the length of SEQ ID NO:3.

### Embodiment 111

The method of any one of embodiments 101-109, wherein the MANF family protein has a length that is 100% the length of SEQ ID NO:3.

### Embodiment 112

The method of embodiment 83, wherein the MANF family protein consists of a sequence listed in Table 4.

### Embodiment 113

The method of embodiment 112, wherein the MANF family protein is cell permeable.

### Embodiment 114

The method of any one of embodiments 83-113, wherein the pharmaceutical composition is administered to an eye of the subject.

### Embodiment 115

The method of embodiment 114, wherein the pharmaceutical composition is administered by topical administration, intravitreal injection, intracameral administration, subconjunctival administration, subtenon administration, retrobulbar administration, posterior juxtascleral administration, or a combination thereof.

### Embodiment 116

The method of embodiment 114, wherein the pharmaceutical composition is administered by topical administration.

### Embodiment 117

The method of embodiment 114, wherein the pharmaceutical composition is administered by intravitreal injection.

### Embodiment 118

The method of any one of embodiments 83-117, wherein the dose has a volume of about: 1-500 μL, 10-250 μL, 25-150 μL, 50-100 μL, 1 μL, 5 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 105 μL, 110 μL, 115 μL, 120 μL, 125 μL, 150 μL, 175 μL, 200 μL, 225 μL, 250 μL, 300 μL, 350 μL, 400 μL, 450 μL, or 500 μL.

### Embodiment 119

The method of any one of embodiments 83-117, wherein the dose has a volume of from about 1 μL to about 500 μL.

### Embodiment 120

The method of any one of embodiments 83-117, wherein the dose has a volume of from about 10 μL to about 250 μL.

### Embodiment 121

The method of any one of embodiments 83-117, wherein the dose has a volume of about 25 μL to about 150 μL.

### Embodiment 122

The method of any one of embodiments 83-117, wherein the dose has a volume of from about 50 μL to about 100 μL.

### Embodiment 123

The method of any one of embodiments 83-117, wherein the dose has a volume of from about 25 μL, to about 125 μL.

### Embodiment 124

The method of any one of embodiments 83-117, wherein the dose has a volume of about 50 μL.

### Embodiment 125

The method of any one of embodiments 83-117, wherein the dose has a volume of about 100 μL.

### Embodiment 126

The method of any one of embodiments 83-125, wherein the dose has a concentration of the MANF family protein that is about: 0.1-100 mg/mL, 0.1-40 mg/mL, 1-20 mg/mL, 1.5-15 mg/mL, 8.1-32.4 mg/mL, 2.7-5.4 mg/mL, 0.1 mg/mL, 0.5 mg/mL, 1 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 ng/mL, 2 ng/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3 mg/mL, 3.1 mg/mL, 3.2 mg/mL, 3.3 mg/mL, 3.4 mg/mL, 3.5 mg/mL, 3.6 mg/mL, 3.7 mg/mL, 3.8 mg/mL, 3.9 mg/mg/mL, 4 mg/mL, 4.1 mg/mL, 4.2 mg/mL, 4.3 mg/mL, 4.4 mg/mL, 4.5 ing/mL, 46 mg/mL, 4.7 mg/mL, 4.8 mg/mL, 4.9 mg/mL, 5 mg/mL, 5.1 mg/mL, 5.2 mg/mL, 5.3 mg/mL, 5.4 mg/mL, 5.5 mg/mL, 5.6 mg/mL, 5.7 mg/mL, 5.8 mg/mL, 5.9 mg/mL, 6 mg/mL, 6.25 mg/mL, 6.5 mg/mL, 6.75 mg/mL, 7 mg/mL, 7.25 mg/mL, 7.5 mg/mL, 7.75 mg/mL, 8 mg/mL, 8.25 mg/mL, 8.5 mg/mL, 8.75 mg/mL, 9 mg/mL, 9.25 mg/mL, 9.5 mg/mL, 9.75 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 ng/mL, 20 mg/mL, 25 mg/mL, 30 mg/mL, 35 mg/mL, 40 mg/mL, 50 mg/mL, 60 mg/mL, 70 mg/mL, 80 mg/mL, 90 mg/mL, or 100 mg/mL.

### Embodiment 127

The method of any one of embodiments 83-125, wherein the dose has a concentration of the MANF family protein that is from about 1 mg/mL to about 20 mg/mL.

### Embodiment 128

The method of any one of embodiments 83-125, wherein the dose has a concentration of the MANF family protein that is from about 1.5 mg/mL to about 15 mg/mL.

### Embodiment 129

The method of any one of embodiments 83-125, wherein the dose has a concentration of the MANF family protein that is from about 2.7 mg/mL to about 5.4 mg/mL.

### Embodiment 130

The method of any one of embodiments 83-129, wherein the effective amount of the MANF family protein is about: 1-5000 μg, 5-2500 μg, 10-2000 μg, 500-2000 mg/mL, 50-1000 μg, 100-500 μg, 200-350 μg, 250-300 μg, 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 525 μg, 550 μg, 575 μg, 600 μg, 625 μg, 650 μg, 675 μg, 700 μg, 750 μg, 800 μg, 850 μg, 900 μg, 950 μg, 1000 μg, 1100 μg, 1200 μg, 1300 μg, 1400 μg, 1500 μg, 1750 μg, 2000 μg, 2500 μg, 3000 μg, 3500 μg, 4000 μg or 5000 μg.

### Embodiment 131

The method of any one of embodiments 83-129, wherein the effective amount of the MANF family protein is from about 50 μg to about 1000 Mg.

### Embodiment 132

The method of any one of embodiments 83-129, wherein the effective amount of the MANF family protein is from about 100 Mg to about 500 t g.

### Embodiment 133

The method of any one of embodiments 83-129, wherein the effective amount of the MANF family protein is from about 250 μg to about 300 μg.

### Embodiment 134

The method of any one of embodiments 83-133, wherein the dose is administered once every 2 to 8 weeks.

### Embodiment 135

The method of any one of embodiments 83-133, wherein the dose is administered once every 3 to 6 weeks.

### Embodiment 136

The method of any one of embodiments 83-133, wherein the dose is administered once every month.

### Embodiment 137

The method of any one of embodiments 83-133, wherein the dose is administered once every two months.

### Embodiment 138

The method of any one of embodiments 83-133, wherein the dose is administered once every hour.

### Embodiment 139

The method of any one of embodiments 83-133, wherein the dose is administered once every two hours.

### Embodiment 140

The method of any one of embodiments 83-133, wherein the dose is administered once every four hours.

### Embodiment 141

The method of any one of embodiments 83-133, wherein the dose is administered daily.

### Embodiment 142

The method of any one of embodiments 83-133, wherein the dose is only administered once.

### Embodiment 143

The method of any one of embodiments 83-133, wherein the retinal artery occlusion is an acute retinal artery occlusion.

### Embodiment 144

The method of any one of embodiments 83-143, wherein the retinal artery occlusion is a central retinal artery occlusion.

### Embodiment 145

The method of any one of embodiments 83-143, wherein the retinal artery occlusion is a branch retinal artery occlusion.

### Embodiment 146

A method of treating a retinal disorder, the method comprising administering to a subject in need thereof an effective amount of a MANF family protein and another active agent.

### Embodiment 147

The method of embodiment 146, wherein the MANF family protein and the another active agent have a synergistic effect upon retinal ganglion cell survival.

### Embodiment 148

The method of any one of embodiments 146-147, wherein the MANF family protein and the another active agent exhibit therapeutic synergy.

### Embodiment 149

The method of any one of embodiments 146-148, wherein the MANF family protein and the another active agent have additive effects.

### Embodiment 150

The method of any one of embodiments 146-149, wherein the MANF family protein is MANF, or a fragment thereof.

### Embodiment 151

The method of any one of embodiments 146-150, wherein the MANF family protein is CDNF, or a fragment thereof.

### Embodiment 152

The method of any one of embodiments 146-151, wherein the another active agent is a prostaglandin analog, a beta-adrenergic receptor antagonist, an alpha adrenergic agonist, a miotic agent, a carbonic anhydrase inhibitor, or a combination thereof.

### Embodiment 153

The method of any one of embodiments 146-152, wherein the another active agent is a prostaglandin analog.

### Embodiment 154

The method of embodiment 153, wherein the prostaglandin analog is latanoprost, bimatoprost, travoprost, unoprostone, or a pharmaceutical salt thereof, or any combination thereof.

### Embodiment 155

The method of any one of embodiments 146-154, wherein the another active agent is a beta-adrenergic receptor antagonist.

### Embodiment 156

The method of embodiment 155, wherein the beta-adrenergic receptor antagonist is betaxolol, carteolol, levobunolol, metipranolol, timolol, or a pharmaceutical salt thereof, or any combination thereof.

### Embodiment 157

The method of any one of embodiments 146-156, wherein the another active agent is an alpha adrenergic agonist.

### Embodiment 158

The method of embodiment 157, wherein the alpha adrenergic agonist is an α1 adrenergic agonist, an α2-adrenergic agonist, or any combination thereof.

### Embodiment 159

The method of embodiment 158, comprising the ac adrenergic agonist that is methoxamine, methylnorepinephrine, midodrine, oxymetazoline, metaraminol, phenylephrine, or a pharmaceutical salt thereof or any combination thereof.

### Embodiment 160

The method of embodiment 158, comprising the α2 adrenergic agonist that is clonidine, guanfacine, guanabenz, guanoxabenz, guanethidine, xylazine, tizanidine, methyldopa, fadolmidine, dexmedetomidine, brimonidine, or a pharmaceutical salt thereof, or any combination thereof.

### Embodiment 161

The method of embodiment 157, wherein the alpha adrenergic agonist is methoxamine, methylnorepinephrine, midodrine, oxymetazoline, metaraminol, phenylephrine, clonidine, guanfacine, guanabenz, guanoxabenz, guanethidine, xylazine, tizanidine, methyldopa, fadolmidine, dexmedetomidine, amidephrine, amitraz, anisodamine, apraclonidine, brimonidine, cirazoline, detomidine, dexmedetomidine, epinephrine, ergotamine, etilefrine, indanidine, lofexidine, medetomidine, mephentermine, metaraminol, methoxamine, mivazerol, naphazoline, norepinephrine, norfenefrine, octopamine, oxymetazoline, phenylpropanolamine, rilmnenidine, romifidine, synephrine, talipexole, or a pharmaceutical salt thereof, or any combination thereof.

### Embodiment 162

The method of any one of embodiments 146-161, wherein the another active agent is an α1 adrenergic agonist that is methoxamine, methylnorepinephrine, midodrine, oxymetazoline, metaraminol, phenylephrine, or a pharmaceutical salt thereof, or any combination thereof.

### Embodiment 163

The method of any one of embodiments 146-162, wherein the another active agent is an α2 adrenergic agonist that is clonidine, guanfacine, guanabenz, guanoxabenz, guanethidine, xylazine, tizanidine, methyldopa, fadolmhnidine, dexmedetomidine, brimonidine, or a pharmaceutical salt thereof, or any combination thereof.

### Embodiment 164

The method of any one of embodiments 146-163, wherein the another active agent is brimonidine or a pharmaceutical salt thereof.

### Embodiment 165

The method of any one of embodiments 146-164, wherein the MANF family protein is MANF, or a fragment thereof and the other active agent is brimonidine or a pharmaceutical salt thereof.

### Embodiment 166

The method of embodiment 165, wherein the MANF and the brimonidine or a pharmaceutical salt thereof have a synergistic effect upon retinal ganglion cell survival.

### Embodiment 167

The method of embodiment 165, wherein the MANF and the brimonidine or a pharmaceutical salt thereof exhibit therapeutic synergy.

### Embodiment 168

The method of any one of embodiments 146-167, wherein the retinal disorder is an acute retinal artery occlusion.

### Embodiment 169

The method of any one of embodiments 146-167, wherein the retinal disorder is a central retinal artery occlusion or a branch retinal artery occlusion.

### Embodiment 170

The method of any one of embodiments 146-167, wherein the retinal disorder is retinal ischemia.

### Embodiment 171

The method of embodiment 170, wherein the retinal ischemia is caused by glaucoma, carotid artery stenosis, Takayasu's arteritis, giant cell arteritis, thromboembolism, central retinal artery occlusion, central retinal vein occlusion, diabetes, or a combination thereof.

### Embodiment 172

The method of any one of embodiments 146-171, wherein the retinal disorder is macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangioctasia, marfan syndrome, optic neuritis, photokeratitis, retinitis pigmentosa, retinopathy of prematurity, stargardt disease, usher syndrome, or any combination thereof.

### Embodiment 173

The method of embodiment 172, wherein the retinal disorder is age-related macular degeneration that is dry age related macular degeneration or wet age related macular degeneration.

### Embodiment 174

The method of any one of embodiments 146-173, wherein the MANF family protein and the another active agent are administered separately.

### Embodiment 175

The method of any one of embodiments 146-174, wherein the MANF family protein and the another active agent are administered together.

### Embodiment 176

The method of any one of embodiments 146-175, wherein the MANF family protein is administered orally, parenterally, intranasally, or intravenously.

### Embodiment 177

The method of any one of embodiments 146-176, wherein the another active agent is administered orally, parenterally, intranasally, or intravenously.

### Embodiment 178

The method of any one of embodiments 146-177, wherein administration of the MANF family protein is topical, subconjunctival, intravitreal, retrobulbar, intracameral, systemic, or a combination thereof.

### Embodiment 179

The method of any one of embodiments 146-178, wherein administration of the another active agent is topical, subconjunctival, intravitreal, retrobulbar, intracameral, systemic, or a combination thereof.

### Embodiment 180

The method of any one of embodiments 146-179, wherein the effective amount of the MANF family protein is about: 0.5 μg-2.5 μg, 0.5 μg-5 μg, 0.5 μg-7.5 μg, 0.5 μg-12.5 μg, 0.5 μg-25 μg, 0.5 μg-50 μg, 0.5 μg-75 μg, 0.5 μg-100 μg, 0.5 μg-150 μg, 0.5 μg-250 μg, 0.5 μg-500 μg, 0.5 μg-1000 μg, 0.5 μg-1250 μg, 0.5 μg-2500 μg, 2.5 μg-5 μg, 2.5 μg-7.5 μg, 2.5 μg-12.5 μg, 2.5 μg-25 μg, 2.5 μg-50 μg, 2.5 μg-75 μg, 2.5 μg-100 μg, 2.5 μg-150 μg, 2.5 μg-250 μg, 2.5 μg-500 μg, 2.5 μg-1000 μg, 2.5 μg-1250 μg, 2.5 μg-2500 μg, 5 μg-7.5 μg, 5 μg-12.5 μg, 5 μg-25 μg, 5 μg-50 μg, 5 μg-75 μg, 5 μg-100 μg, 5 μg-150 μg, 5 μg-250 μg, 5 μg-500 μg, 5 μg-1000 μg, 5 μg-1250 μg, 5 μg-2500 μg, 7.5 μg-12.5 μg, 7.5 μg-25 μg, 7.5 μg-50 μg, 7.5 μg-75 μg, 7.5 μg-100 μg, 7.5 μg-150 μg, 7.5 μg-250 μg, 7.5 μg-500 μg, 7.5 μg-50000 μg, 7.5 μg-1250 μg, 7.5 μg-2500 μg, 12.5 μg-25 μg, 12.5 μg-50 μg, 12.5 μg-75 μg, 12.5 μg-100 μg, 12.5 μg-150 μg, 12.5 μg-250 μg, 12.5 μg-500 μg, 12.5 μg-1000 μg, 12.5 μg-1250 μg, 12.5 μg-2500 μg, 25 μg-50 μg, 25 μg-75 μg, 25 μg-100 μg, 25 μg-150 μg, 25 μg-250 μg, 25 μg-500 μg, 25 μg-1000 μg, 25 μg-1250 μg, 25 μg-2500 μg, 50 μg-75 μg, 50 μg-100 μg, 50 μg-150 μg, 50 μg-250 μg, 50 μg-500 μg, 50 μg-1000 μg, 50 μg-1250 μg, 50 μg-2500 μg, 75 μg-100 μg, 75 μg-150 μg, 75 μg-250 μg, 75 μg-500 μg, 75 μg-1000 μg, 75 μg-1250 μg, 75 μg-2500 μg, 100 μg-150 μg, 100 μg-250 μg, 100 μg-500 μg, 100 μg-1000 μg, 100 μg-1250 μg, 100 μg-2500 μg, 150 μg-250 μg, 150 μg-500 μg, 150 μg-1000 μg, 150 μg-1250 μg, 150 μg-2500 μg, 250 μg-500 μg, 250 μg-1000 μg, 250 μg-1250 μg, 250 μg-2500 μg, 500 μg-1000 μg, 500 μg-1250 μg, 500 μg-2500 μg, 1000 μg-1250 μg, 1000 μg-2500 μg, or 1250 μg-2500 μg per eye.

### Embodiment 181

The method of any one of embodiments 146-180, wherein the effective amount of the MANF family protein is at least about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 182

The method of any one of embodiments 146-181, wherein the effective amount of the MANF family protein is less than about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 183

The method of any one of embodiments 146-182, wherein the effective amount of the another active agent is about: 0.5 μg-2.5 μg, 0.5 μg-5 μg, 0.5 μg-7.5 μg, 0.5 μg-12.5 μg, 0.5 μg-25 μg, 0.5 μg-50 μg, 0.5 μg-75 μg, 0.5 μg-10 μg, 0.5 μg-150 μg, 0.5 μg-250 μg, 0.5 μg-500 μg, 0.5 μg-1000 μg, 0.5 μg-1250 μg, 0.5 μg-2500 μg, 2.5 μg-5 μg, 2.5 μg-70.5 μg, 2.5 μg-12.5 μg, 2.5 μg-25 μg, 2.5 μg-50 μg, 2.5 μg-75 μg, 2.5 μg-100 μg, 2.5 μg-150 μg, 2.5 μg-250 μg, 2.5 μg-500 μg, 2.5 μg-1000 μg, 2.5 μg-1250 μg, 2.5 μg-2500 μg, 5 μg-7.5 μg, 5 μg-12.5 μg, 5 μg-25 μg, 5 μg-50 μg, 5 μg-75 μg, 5 μg-100 μg, 5 μg-150 μg, 5 μg-250 μg, 5 μg-500 μg, 5 μg-1000 μg, 5 μg-1250 μg, 5 μg-2500 μg, 7.5 μg-12.5 μg, 7.5 μg-25 μg, 7.5 μg-50 μg, 7.5 μg-75 μg, 7.5 μg-100 μg, 7.5 μg-150 μg, 7.5 μg-250 μg, 7.5 μg-500 μg, 7.5 μg-1000 μg, 7.5 μg-1250 μg, 7.5 μg-2500 μg, 12.5 μg-25 μg, 12.5 μg-50 μg, 12.5 μg-75 μg, 12.5 μg-100 μg, 12.5 μg-150 μg, 12.5 μg-250 μg, 12.5 μg-500 μg, 12.5 μg-1000 μg, 12.5 μg-1250 μg, 12.5 μg-2500 μg, 25 μg-50 μg, 25 μg-75 μg, 25 μg-100 μg, 25 μg-150 μg, 25 μg-250 μg, 25 μg-500 μg, 25 μg-1000 μg, 25 μg-1250 μg, 25 μg-2500 μg, 50 μg-75 μg, 50 μg-100 μg, 50 μg-150 μg, 50 μg-250 μg, 50 μg-500 μg, 50 μg-1000 μg, 50 μg-1250 μg, 50 μg-2500 μg, 75 μg-100 μg, 75 μg-150 μg, 75 μg-250 μg, 75 μg-500 μg, 75 μg-1000 μg, 75 μg-1250 μg, 75 μg-2500 μg, 100 μg-150 μg, 100 μg-250 μg, 100 μg-500 μg, 100 μg-1000 μg, 100 μg-1250 μg, 100 μg-2500 μg, 150 μg-250 μg, 150 μg-500 μg, 150 μg-1000 μg, 150 μg-1250 μg, 150 μg-2500 μg, 250 μg-500 μg, 250-500 μg, 250 μg-1000 μg, 250 μg-1250 μg, 250 μg-2500 μg, 500 μg-1000 μg, 500 μg-1250 μg, 500 μg-2500 μg, 1000 μg-1250 μg, 1000 μg-2500 μg, or 1250 μg-2500 μg per eye.

### Embodiment 184

The method of any one of embodiments 146-183, wherein the effective amount of the another active agent is at least about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 185

The method of any one of embodiments 146-184, wherein the effective amount of the another active agent is less than about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 186

The method of any one of embodiments 146-185, wherein the MANF family protein is administered once every 2 to 8 weeks.

### Embodiment 187

The method of any one of embodiments 146-185, wherein the MANF family protein is administered once every 3 to 6 weeks.

### Embodiment 188

The method of any one of embodiments 146-185, wherein the MANF family protein is administered once every month.

### Embodiment 189

The method of any one of embodiments 146-185, wherein the MANF family protein is administered once every two months.

### Embodiment 190

The method of any one of embodiments 146-185, wherein the MANF family protein is administered once every hour.

### Embodiment 191

The method of any one of embodiments 146-185, wherein the MANF family protein is administered once every two hours.

### Embodiment 192

The method of any one of embodiments 146-185, wherein the MANF family protein is administered once every four hours.

### Embodiment 193

The method of any one of embodiments 146-185, wherein the MANF family protein is administered daily.

### Embodiment 194

The method of any one of embodiments 146-185, wherein the MANF family protein is administered only once.

### Embodiment 195

The method of any one of embodiments 146-185, wherein the MANF family protein is administered one, two, or three times per day.

### Embodiment 196

The method of any one of embodiments 146-195, wherein the another active agent is administered once every 2 to 8 weeks.

### Embodiment 197

The method of any one of embodiments 146-195, wherein the another active agent is administered once every 3 to 6 weeks.

### Embodiment 198

The method of any one of embodiments 146-195, wherein the another active agent is administered once every month.

### Embodiment 199

The method of any one of embodiments 146-195, wherein the another active agent is administered once every two months.

### Embodiment 200

The method of any one of embodiments 146-195, wherein the another active agent is administered once every hour.

### Embodiment 201

The method of any one of embodiments 146-195, wherein the another active agent is administered once every two hours.

### Embodiment 202

The method of any one of embodiments 146-195, wherein the another active agent is administered once every four hours.

### Embodiment 203

The method of any one of embodiments 146-195, wherein the another active agent is administered daily.

### Embodiment 204

The method of any one of embodiments 146-195, wherein the another active agent is administered only once.

### Embodiment 205

The method of any one of embodiments 146-195, wherein the another active agent is administered one, two, or three times per day.

### Embodiment 206

A pharmaceutical composition comprising an amount of a MANF family protein and another active agent that is effective for treating a retinal disorder.

### Embodiment 207

The pharmaceutical composition of embodiment 206, further comprising one or more pharmaceutically acceptable excipients.

### Embodiment 208

The pharmaceutical composition of any one of embodiments 206-207, wherein the MANF family protein and the another active agent have a synergistic effect upon retinal ganglion cell survival.

### Embodiment 209

The pharmaceutical composition of any one of embodiments 206-208, wherein the MANE family protein and the another active agent exhibit therapeutic synergy.

### Embodiment 210

The pharmaceutical composition of any one of embodiments 206-209, wherein the MANE family protein and the another active agent have additive effects.

### Embodiment 211

The pharmaceutical composition of any one of embodiments 206-210, wherein the MANF family protein is MANF, or a fragment thereof.

### Embodiment 212

The pharmaceutical composition of any one of embodiments 206-211, wherein the MANF family protein is CDNF, or a fragment thereof.

### Embodiment 213

The pharmaceutical composition of any one of embodiments 206-212, wherein the another active agent is a prostaglandin analog, a beta-adrenergic receptor antagonist, an alpha adrenergic agonist, a miotic agent, a carbonic anhydrase inhibitor, or a combination thereof.

### Embodiment 214

The pharmaceutical composition of any one of embodiments 206-213, wherein the another active agent is a prostaglandin analog.

### Embodiment 215

The pharmaceutical composition of embodiment 214, wherein the prostaglandin analog is latanoprost, bimatoprost, travoprost, unoprostone, or a pharmaceutical salt thereof, or any combination thereof.

### Embodiment 216

The pharmaceutical composition of any one of embodiments 206-215, wherein the another active agent is a beta-adrenergic receptor antagonist.

### Embodiment 217

The pharmaceutical composition of embodiment 216, wherein the beta-adrenergic receptor antagonist is betaxolol, carteolol, levobunolol, metipranolol, timolol, or a pharmaceutical salt thereof, or any combination thereof.

### Embodiment 218

The pharmaceutical composition of any one of embodiments 206-217, wherein the another active agent is an alpha adrenergic agonist.

### Embodiment 219

The pharmaceutical composition of embodiment 218, wherein the alpha adrenergic agonist is an α1 adrenergic agonist, an α2-adrenergic agonist, or any combination thereof.

### Embodiment 220

The pharmaceutical composition of embodiment 219, comprising the α1 adrenergic agonist that is methoxamine, methylnorepinephrine, midodrine, oxymetazoline, metaraminol, phenylephrine, or a pharmaceutical salt thereof, or any combination thereof.

### Embodiment 221

The pharmaceutical composition of embodiment 219, comprising the α2 adrenergic agonist that is clonidine, guanfacine, guanabenz, guanoxabenz, guanethidine, xylazine, tizanidine, methyldopa, fadolimidine, dexmedetornmidine, brirnonidine, or a pharmaceutical salt thereof, or any combination thereof.

### Embodiment 222

The pharmaceutical composition of embodiment 218, wherein the alpha adrenergic agonist is methoxamine, methylnorepinephrine, midodrine, oxymetazoline, metaraminol, phenylephrine, clonidine, guanfacine, guanabenz, guanoxabenz, guanethidine, xylazine, tizanidine, methyldopa, fadolmidine, dexmedetomidine, amidephrine, amitraz, anisodamine, apraclonidine, brimonidine, cirazoline, detomidine, dexmedetomidine, epinephrine, ergotamine, etilefrine, indanidine, lofexidine, medetomidine, mephentermine, metaramninol, methoxamine, mivazerol, naphazoline, norepinephrine, norfenefrine, octopamine, oxymetazoline, phenylpropanolamine, rilmenidine, romifidine, synephrine, talipexole, or a pharmaceutical salt thereof, or any combination thereof.

### Embodiment 223

The pharmaceutical composition of any one of embodiments 206-222, wherein the another active agent is an α1 adrenergic agonist that is methoxamine, methylnorepinephrine, midodrine, oxymetazoline, metaraminol, phenyliephrine, or a pharmaceutical salt thereof, or any combination thereof.

### Embodiment 224

The pharmaceutical composition of any one of embodiments 206-223, wherein the another active agent is an α2 adrenergic agonist that is clonidine, guanfacine, guanabenz, guanoxabenz, guanethidine, xylazine, tizanidine, methyldopa, fadolmidine, dexmedetomidine, brimonidine, or a pharmaceutical salt thereof, or any combination thereof.

### Embodiment 225

The pharmaceutical composition of any one of embodiments 206-224, wherein the another active agent is brimonidine or a pharmaceutical salt thereof.

### Embodiment 226

The pharmaceutical composition of any one of embodiments 206-225, wherein the MANF family protein is MANF, or a fragment thereof and the other active agent is brimonidine or a pharmaceutical salt thereof.

### Embodiment 227

The pharmaceutical composition of embodiment 226, wherein the MANF and the brimonidine or a pharmaceutical salt thereof have a synergistic effect upon retinal ganglion cell survival.

### Embodiment 228

The pharmaceutical composition of embodiment 226, wherein the MANF and the brirnonidine or a pharmaceutical salt thereof exhibit therapeutic synergy.

### Embodiment 229

The pharmaceutical composition of any one of embodiments 206-228, wherein the retinal disorder is retinal ischemia.

### Embodiment 230

The pharmaceutical composition of embodiment 229, wherein the retinal ischemia is caused by glaucoma, carotid artery stenosis, Takayasu's arteritis, giant cell arteritis, thromboembolism, central retinal artery occlusion, central retinal vein occlusion, diabetes, or a combination thereof:

### Embodiment 231

The pharmaceutical composition of any one of embodiments 206-230, wherein the retinal disorder is macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangioctasia, marfan syndrome, optic neuritis, photokeratitis, retinitis pigmentosa, retinopathy of prematurity, stargardt disease, usher syndrome, or any combination thereof.

### Embodiment 232

The pharmaceutical composition of embodiment 231, wherein the retinal disorder is age-related macular degeneration that is dry age related macular degeneration or wet age related macular degeneration.

### Embodiment 233

The pharmaceutical composition of any one of embodiments 206-232, wherein the MANF family protein and the another active agent are administered separately.

### Embodiment 234

The pharmaceutical composition of any one of embodiments 206-233, wherein the MANF family protein and the another active agent are administered together.

### Embodiment 235

The pharmaceutical composition of any one of embodiments 206-234, wherein the MANF family protein is administered orally, parenterally, intranasally, or intravenously.

### Embodiment 236

The pharmaceutical composition of any one of embodiments 206-235, wherein the another active agent is administered orally, parenterally, intranasally, or intravenously.

### Embodiment 237

The pharmaceutical composition of any one of embodiments 206-236, wherein administration of the MANF family protein is topical, subconjunctival, intravitreal, retrobulbar, intracameral, systemic, or a combination thereof.

### Embodiment 238

The pharmaceutical composition of any one of embodiments 206-237, wherein administration of the another active agent is topical, subconjunctival, intravitreal, retrobulbar, intracameral, systemic, or a combination thereof.

### Embodiment 239

The pharmaceutical composition of any one of embodiments 206-238, wherein the effective amount of the MANF family protein is about: 0.5 μg-2.5 μg, 0.5 μg-5 μg, 0.5 μg-7.5 μg, 0.5 μg-12.5 μg, 0.5 μg-25 μg, 0.5 μg-50 μg, 0.5 μg-75 μg, 0.5 μg-100 μg, 0.5 μg-0.5 μg, 0.5 μg, 0.5 μg-500 μg, 0.5 μg-500 μg, 0.5 μg-1000 μg, 0.5 μg-1250 μg, 0.5 μg-2500 μg, 2.5 μg-5 μg, 2.5 μg-7.5 μg, 2.5 μg-12.5 μg, 2.5 μg-25 μg, 2.5 μg-50 μg, 2.5 μg-75 μg, 2.5 μg-100 μg, 2.5 μg-150 μg, 2.5 μg-250 μg, 2.5 μg-500 μg, 2.5 μg-1000 μg, 2.5 μg-1250 μg, 2.5 μg-2500 μg, 5 μg-7.5 μg, 5 μg-12.5 μg, 5 μg-25 μg, 5 μg-50 μg, 5 μg-75 μg, 5 μg-100 μg, 5 μg-150 μg, 5 μg-250 μg, 5 μg-500 μg, 5 μg-1000 μg, 5 μg-1250 μg, 5 μg-2500 μg, 7.5 μg-12.5 μg, 7.5 μg-25 μg, 7.5 μg-50 μg, 7.5 μg-75 μg, 7.5 μg-100 μg, 7.5 μg-150 μg, 7.5 μg-250 μg, 7.5 μg-500 μg, 7.5 μg-1000 μg, 7.5 μg-1250 μg, 7.5 μg-2500 μg, 12.5 μg-25 μg, 12.5 μg-50 μg, 12.5 μg-75 μg, 12.5 μg-100 μg, 12.5 μg-150 μg, 12.5 μg-250 μg, 12.5 μg-500 μg, 12.5 μg-1000 μg, 12.5 μg-1250 μg, 12.5 μg-2500 μg, 25 μg-50 μg, 25 μg-75 μg, 25 μg-100 μg, 25 μg-150 μg, 25 μg-250 μg, 25 μg-500 μg, 25 μg-1000 μg, 25 μg-1250 μg, 25 μg-2500 μg, 50 μg-75 μg, 50 μg-100 μg, 50 μg-150 μg, 50 μg-250 μg, 50 μg-500 μg, 50 μg-1000 μg, 50 μg-1250 μg, 50 μg-2500 μg, 75 μg-100 μg, 75 μg-150 μg, 75 μg-250 μg, 75 μg-500 μg, 75 μg-1000 μg, 75 μg-1250 μg, 75 μg-2500 μg, 100 μg-150 μg, 100 μg-250 μg, 100 μg-500 μg, 100 μg-1000 μg, 100 μg-1250 μg, 100 μg-2500 μg, 150 μg-250 μg, 150 μg-500 μg, 150 μg-1000 μg, 150 μg-1250 μg, 150 μg-2500 μg, 250 μg-500 μg, 250 μg-1000 μg, 250 μg-1250 μg, 250 μg-2500 μg, 500 μg-1000 μg, 500 μg-1250 μg, 500 μg-2500 μg, 1000 μg-1250 μg, 1000 μg-2500 μg, or 1250 μg-2500 μg per eye.

### Embodiment 240

The pharmaceutical composition of any one of embodiments 206-239, wherein the effective amount of the MANF family protein is at least about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 241

The pharmaceutical composition of any one of embodiments 206-240, wherein the effective amount of the MANF family protein is less than about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 242

The pharmaceutical composition of any one of embodiments 206-241, wherein the effective amount of the another active agent is about: 0.5 μg-2.5 μg, 0.5 μg-5 μg, 0.5 μg-7.5 μg, 0.5 μg-12.5 μg, 0.5 μg-25 pug, 0.5 μg-50 μg, 0.5 μg-75 μg, 0.5 μg-100 μg, 0.5 μg-150 μg, 0.5 μg-250 μg, 0.5 μg-500 μg, 0.5 μg-1000 μg, 0.5 μg-1250 μg, 0.5 μg-2500 μg, 2.5 μg-5 μg, 2.5 μg-7.5 μg, 2.5 μg-12.5 μg, 2.5 μg-25 μg, 2.5 μg-50 μg, 2.5 μg-75 μg, 2.5 μg-100 μg, 2.5 μg-150 μg, 2.5 μg-250 μg, 2.5 μg-500 μg, 2.5 μg-1000 μg, 2.5 μg-1250 μg, 2.5 μg-2500 μg, 5 μg-7.5 μg, 5 μg-12.5 μg, 5 μg-25 μg, 5 μg-50 μg, 5 μg-75 μg, 5 μg-100 μg, 5 μg-150 μg, 5 μg-250 μg, 5 μg-100 μg, 5 μg-1000 μg, 55 μg-1250 μg, 5 μg-25 μg-5 μg-2500 μg, 7.5 μg-12.5 μg, 7.5 μg-25 μg, 7.5 μg-50 μg, 7.5 μg-75 μg, 7.5 μg-100 μg, 7.5 μg-150 μg, 7.5 μg-250 μg, 7.5 μg-500 μg, 7.5 μg-1000 μg, 7.5 μg-1250 μg, 7.5 μg-2500 μg, 12.5 μg-25 μg, 12.5 μg-50 μg, 12.5 μg-75 μg, 12.5 μg-100 μg, 12.5 μg-150 μg, 12.5 μg-250 μg, 12.5 μg-500 μg, 12.5 μg-1000 μg, 12.5 μg-1250 μg, 12.5 μg-2500 μg, 25 μg-50 μg, 25 μg-75 μg, 25 μg-100 μg, 25 μg-150 μg, 25 μg-250 μg, 25 μg-500 μg, 25 μg-1000 μg, 25 μg-1250 μg, 25 μg-2500 μg, 50 μg-75 μg, 50 μg-100 μg, 50 μg-150 μg, 50 μg-250 μg, 50 μg, 50 μg, 50 μg-1000 μg, 50 μg-1250 μg, 50 μg-2500 μg, 75 μg-100 μg, 75 μg-150 μg, 75 μg-250 μg, 75 μg-500 μg, 75 μg-1000 μg, 75 μg-1250 μg, 75 μg-2500 μg, 100 μg-150 μg, 100 μg-250 μg, 100 μg-500 μg, 100 μg-1000 μg, 100 μg-1250 μg, 100 μg-2500 μg, 150 μg-250 μg, 150 μg-500 μg, 150 μg-1000 μg, 150 μg-1250 μg, 150 μg-2500 μg, 250 μg-500 μg, 250 μg-1000 μg, 250 μg-1250 μg, 250 μg-2500 μg, 500 μg-1000 μg, 500 μg-1250 μg, 500 μg, 250 μg-1000 μg-1250 μg, 1000 μg-2500 μg, or 1250 μg-2500 μg per eye.

### Embodiment 243

The pharmaceutical composition of any one of embodiments 206-242, wherein the effective amount of the another active agent is at least about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 244

The pharmaceutical composition of any one of embodiments 206-243, wherein the effective amount of the another active agent is less than about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

### Embodiment 245

The pharmaceutical composition of any one of embodiments 206-244, wherein the MANF family protein is administered once every 2 to 8 weeks.

### Embodiment 246

The pharmaceutical composition of any one of embodiments 206-244, wherein the MANF family protein is administered once every 3 to 6 weeks.

### Embodiment 247

The pharmaceutical composition of any one of embodiments 206-244, wherein the MANF family protein is administered once every month.

### Embodiment 248

The pharmaceutical composition of any one of embodiments 206-244, wherein the MANF family protein is administered once every two months.

### Embodiment 249

The pharmaceutical composition of any one of embodiments 206-244, wherein the MANF family protein is administered once every hour.

### Embodiment 250

The pharmaceutical composition of any one of embodiments 206-244, wherein the MANF family protein is administered once every two hours.

### Embodiment 251

The pharmaceutical composition of any one of embodiments 206-244, wherein the MANF family protein is administered once every four hours.

### Embodiment 252

The pharmaceutical composition of any one of embodiments 206-244, wherein the MANF family protein is administered daily.

### Embodiment 253

The pharmaceutical composition of any one of embodiments 206-244, wherein the MANF family protein is administered only once.

### Embodiment 254

The pharmaceutical composition of any one of embodiments 206-244, wherein the MANF family protein is administered one, two, or three times per day.

### Embodiment 255

The pharmaceutical composition of any one of embodiments 206-254, wherein the another active agent is administered once every 2 to 8 weeks.

### Embodiment 256

The pharmaceutical composition of any one of embodiments 206-254, wherein the another active agent is administered once every 3 to 6 weeks.

### Embodiment 257

The pharmaceutical composition of any one of embodiments 206-254, wherein the another active agent is administered once every month.

### Embodiment 258

The pharmaceutical composition of any one of embodiments 206-254, wherein the another active agent is administered once every two months.

### Embodiment 259

The pharmaceutical composition of any one of embodiments 206-254, wherein the another active agent is administered once every hour.

### Embodiment 260

The pharmaceutical composition of any one of embodiments 206-254, wherein the another active agent is administered once every two hours.

### Embodiment 261

The pharmaceutical composition of any one of embodiments 206-254, wherein the another active agent is administered once every four hours.

### Embodiment 262

The pharmaceutical composition of any one of embodiments 206-254, wherein the another active agent is administered daily.

### Embodiment 263

The pharmaceutical composition of any one of embodiments 206-254, wherein the another active agent is administered only once.

### Embodiment 264

The pharmaceutical composition of any one of embodiments 206-254, wherein the another active agent is administered one, two, or three times per day.

### Embodiment 265

The pharmaceutical composition of any one of embodiments 206-264, provided as an eye drop or an injectable liquid.

### Examples

### Example 1: Production of Human Recombinant MANF

Human MANF is expressed in a pre-form of 179 amino acid residues. The MANF protein is N-terminally processed and a signal sequence of 21 amino acids is removed, yielding the mature, secreted and active MANF with a length of 158 amino acid residues. The mature human MANF protein starts with the amino acid sequence LRPGD . . . and ends with . . . RTDL (See Table 1). In this example, MANF product is the recombinant form of human MANE (i.e., hrMANF) encompassing the mature sequence of 158 amino acid residues.

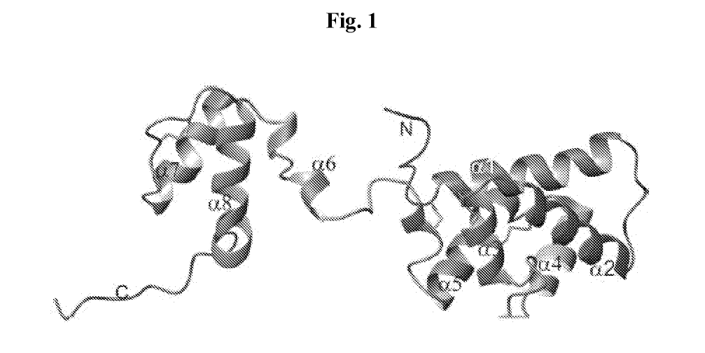

The three-dimensional structure of full-length MANF has been determined by NMR and by X-ray crystallography. The three-dimensional structure of human MANF is shown in FIG. 1. The N-terminal domain (N-domain) of MANF encompassing residues L20-L120 is entirely helical, with four α-helices and a rare structural element, a π helix, immediately followed by a 310 helix. Most m-helices are involved in binding enzyme substrates or ligand molecules. The N-domain contains three disulfide bonds. A cluster of positively charged residues in the π and 310 helices are conserved among MANF homologues.

The C-terminal domain (C-domain) of MANF encompasses residues T126-L158 and is well defined in the NMR solution structure. This domain is also entirely helical and contains one disulfide bond between conserved cysteines in the CXXC motif between α-helices 5 and 6. The CXXC motif is a consensus sequence of proteins of the thiol-protein oxidoreductase superfamily, other members of which include thioredoxins, glutaredoxins, and peroxiredoxins.

The recombinant form of human MANF (hrMANF) was produced using the QMCF technology. The QMCF technology uses an episomal protein expression system that allows for expression of recombinant proteins in mammalian cells over an extended period of time (e.g., up to 50 days). The hrMANF protein was expressed by the QMCF protein production technology using the CHOEBNALT85 suspension cell line over a period of 11 days. The hrMANF was purified from the supernatant of expressing cells using a two-step ion-exchange chromatography and gel filtration (Superdex 75). The final hrMANF storage buffer was phosphate buffered saline (PBS) pH 7.4.

The purity of the expressed hrMANF protein was evaluated by Coomassie-stained SDS-PAGE and Western blot (FIG. 2).

### Example 2: Evaluation of a Single Intravitreal Administration of MANF in a Rat Model of Retinal Ischemia-Induced Ganglion Cell Degeneration

### SUMMARY

Purpose:

The aim of this study was to evaluate the effects of intravitreal administration of MANF at 3 different doses (0.15 mg/mL, 0.5 ing/mL and 1.5 mg/mL) on retinal electric activity and retinal ganglion cell survival after a transient ischemia by clamping in albino rats.

In this Example, retinal ischemia was induced by transient vascular clamping of the optic nerve in the right eyes of Sprague Dawley albino rats for 45 min. Reperfusion was initiated by the release of the clamp. Retinal function was evaluated by ERG at baseline and 7 days after ischemia. RGC survival was assessed by immunohistochemistry 7 days after ischemia. This model is appropriate for the study of acute RAO, but it may not represent chronic retinal artery hypoperfusion.

Methods:

Sixty (60) rats were randomly divided into five (5) groups of twelve (12) animals each.

MANF at 0.15 mg/mL, 0.5 mg/mL and 1.5 mg/mL or vehicle (phosphate buffered saline; PBS) were administered by intravitreal administration (4 μL) in right eyes once, immediately after reperfusion.

Reference (Aphagan®, 1 mg/kg brimonidine) was intraperitoneally dosed once, 30 min before optic nerve clamping.

Retinal ischemia was induced by vascular clamping of the optic nerve in the right eyes for 45 min. Reperfusion was initiated by the release of the clamp. Retinal function was evaluated by electroretinography (ERG) at baseline and 7 days after ischemia. Retinal Ganglion Cell (RGC) survival was assessed by immunohistology 7 days after ischemia.

Results:

General Behavior:

The general behavior and appearance of all animals were not altered by MANF treatment, regardless the dose. All animals showed a normal body weight gain from baseline to Day 7. No abnormal behavior or unhealthy signs were found for any treated animals during the study period. However, 3 animals died during the study: One animal treated with 1 mg/kg brimonidine was found dead on Day 0 after clamping. One animal each treated with 1 mg/kg brimonidine or PBS were found dead on Day 7 before evaluations were performed. These deaths were attributed to the effects of anesthesia and not to the treatment.

Electroretinogram:

The functional status of the retina was evaluated by electroretinography one week after the ischemic insult and compared to baseline values determined just prior to optic nerve ischemia. The b-wave is induced by potassium efflux shunted from activated bipolar cells by Müller cells and is an electrophysiological indicator of inner retinal signal transmission. The ERG parameters applied were the following: Color: white maximum; Maximum intensity: 2.6 cd·s/m2 (0 dB); Duration 0.24 ms; Numbers of flashes: 1; Filter: 50 Hz; Impedance threshold: 90 kΩ.

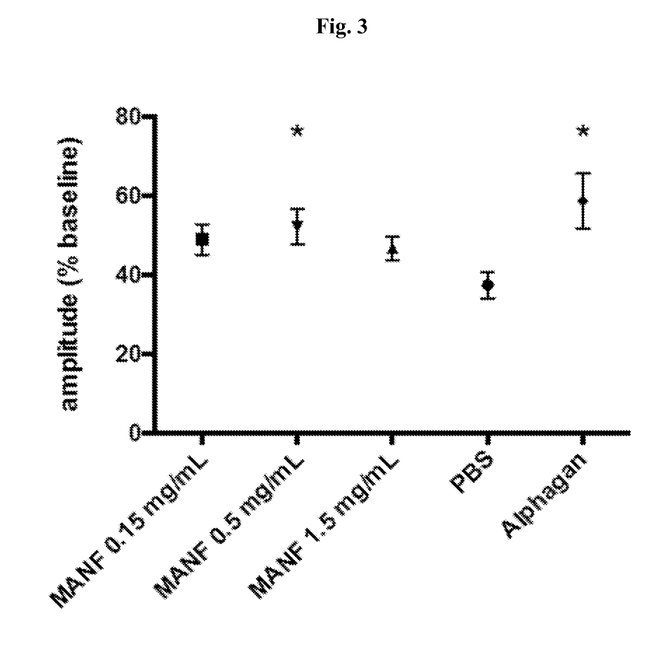

The results are presented in Table 5 and FIG. 3. As shown in Table 5 and FIG. 3, the clamping of the optic nerve for 45 minutes resulted in a marked impairment of the b-wave amplitude and of the RGC density in PBS-treated animals one week after the ischemic insult.

The b-wave amplitude and the RGC survival were improved after single intravitreal administration of MANF, regardless the dose, as well as with 1 mg/kg brimonidine treatment.

A significant improvement of the b-wave recovery was shown after intravitreal administration of MANF at 0.5 mg/mL (p=0.0193, ANOVA followed by Dunn's multiple comparison tests against PBS control). The b-wave amplitude on Day 7 recovered to 52% of the baseline mean value.

A marked but not statistically significant protection from the reduction in b-wave amplitude was observed after intravitreal administration of MANF at 0.15 mg/mL and 1.5 mg/mL, in comparison with the PBS-treated group. The b-wave amplitudes were 49% and 47% of the mean baseline value for the groups treated with MANF at 0.15 mg/mL and 1.5 mg/mL, respectively, while the PBS-treated group displayed a b-wave amplitude of 37% on Day 7 compared to the baseline value.

Prophylactically administered Alphagan® (1 mg/kg brimonidine) led to a significant protection of the b-wave amplitude in comparison with the vehicle group (p=0.015, ANOVA followed by Dunn's multiple comparison tests against PBS control). The b-wave amplitude recovered to 59% of the baseline mean value.

TABLE 5
Normalized b-wave amplitudes and BrN3A-positive
cell density one week after ischemia (right eye)

| | | MANF | MANF | MANF | | |
|---|---|---|---|---|---|---|
| | | 0.15 | 0.5 | 1.5 | | |
| Treatment | | mg/mL | mg/mL | mg/mL | PBS | Alphagan ® |
| Normalized | Mean ± | 49 ± 13% | 52 ± 15% | 47 ± 10% | 37 ± 11% | 59 ± 22% |
| b-wave | SD | | | | | |
| amplitude | | | | | | |
| (% of baseline) | | | | | | |
| RGC density | Mean ± | 403 ± 189 | 465 ± 301 | 488 ± 214 | 264 ± 261 | 578 ± 185 |
| ⁽¹⁾ (cells/mm²) | SD | | | | | |

Note:
⁽¹⁾ non ischemic retina: 2121 ± 455 RGC/mm²

Retinal Ganglion Cell Survival:

To assess the effect of the treatment on RGC viability, the RGC density was evaluated 7 days after ischemia by immunohistochemistry with a stain for the RGC specific marker Brn3a.

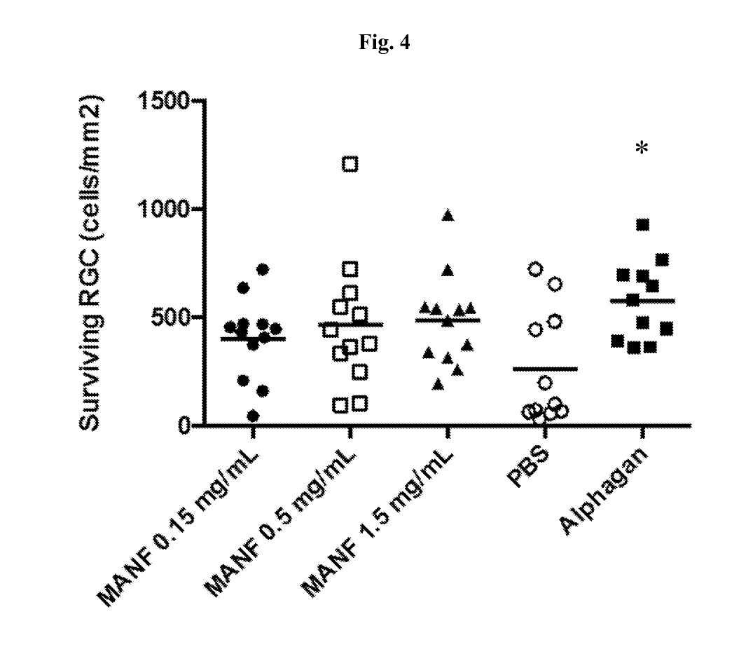

The results are shown in Table 5 and FIG. 4. As shown in Table 5 and FIG. 4, the clamping of the optic nerve for 45 minutes resulted in a marked impairment of the RGC density in PBS-treated animals one week after the ischemic insult.

The RGC density in the retina of non-ischemic eyes (two left eyes from each group) was 2121±455 RGC/mm² (n=10). One week after ischemia, mean RGC density decreased to 264±261 RGC/mm² (−88% compared to non-ischemic eyes) in the PBS-treated group.

Intravitreal administration of MANF at 0.15 mg/mL, 0.5 mg/mL and 1.5 mg/mL, showed a trend in improvement of the mean RGC survival 7 days after injury with 403±189 cells/mm^{z}, 465±301 cells/mm² and 488±214 cells/mm², respectively.

Intraperitoneal administration of Alphagan® (1 mg/kg brimonidine) significantly prevented the decrease of surviving RGCs, with 578±185 RGCs/mm² (p=0.0177, ANOVA followed by Dunn's multiple comparison tests against PBS control), when compared with the PBS-treated group.

### CONCLUSION

Under the experimental conditions, a single intravitreal administration of MANF at 0.15 mg/mL, 0.5 mg/mL and 1.5 mg/mL displayed marked efficacy in preserving retinal function (ERG, b-wave amplitude) and a trend in protecting RGCs after a one-week reperfusion period in a rat model of retinal ischemia with clamping. The effect on the b-wave amplitude observed in the MANF (0.5 mg/mL) group was significantly different than the vehicle (PBS) treated group.

The reference Alphagan® (1 mg/kg brimonidine) showed a significant efficiency in improving retinal function and protecting RGC.

### Introduction

### Background

Retinal ischemia is a common cause of visual impairment and blindness. A number of clinical conditions, including central retinal artery or vein occlusion (CRAO, CRVO), diabetes, or glaucoma make themselves manifest by a reduction of retinal blood supply. Retinal ischemia initiates a self-reinforcing destructive cascade involving neuronal depolarization, calcium influx and oxidative stress initiated by energy failure and increased glutamatergic stimulation. The initial ischemic insult results in cellular perturbations that continue to progress despite or perhaps because of, reperfusion of the ischemic tissue. Ultimately, the retinal ganglion cells (RGC) die via apoptosis.

Many studies have focused attention on histological or biochemical measures of protection of retinal ganglion cells. Demonstration of drug efficacy may also be assessed by measuring retinal function. The functional status of the retina is monitored by electroretinogram (ERG). The b-wave, which is induced by potassium efflux shunted “on” from bipolar cells by Muller cells in response to illumination, is the ERG-component most susceptible to ischemia.

Thus, suppression of the b-wave of the ERG has been taken as an electrophysiological measure of retinal blood flow in humans and in experimental animal models. The a-wave, which is induced by light-activated hyperpolarization of photoreceptors, is usually less affected by changes in blood flow. Retinal protection in this model is also assessed directly by counting RGCs stained with BrN3a.

Several laboratories have shown the capacity of different substances, including α2-adrenergic agonists, to prevent degeneration induced by retinal ischemia. Brimonidine has been demonstrated to show a neuroprotective effect in this model.

Materials

Test Item

TABLE 6
Material table - Test item

| Material | Test item |
|---|---|
| Denomination | MANF |
| Concentration | 3 mg/mL |
| Characteristics (form, aspect, | Injectable solution |
| etc.) | |
| Batch number | 03.05.2013 Icosagen |
| Quantity received | 5 × 1 mL |
| Preparation | 6 μg/eye (1.5 mg/mL)-2 μg/eye |
| | (0.5 mg/mL)-0.6 μg/eye (0.15 mg/mL) |
| | Diluted in PBS |
| Storage conditions and stability | Long: −80 ± 7° C.; In use: at room |
| | temperature |

Control Item

TABLE 7
Material table - Control item

| Material | Control item |
|---|---|
| Denomination | Vehicle (PBS) |
| Characteristics (form, aspect, | Solution |
| molecular weight, etc.) | |
| Reference/Batch number | Sigma: P5368/SLBG9935V |
| Preparation (concentration, vehicle, | One pouch dissolved in 1 L of |
| composition, appearance after | distilled water, was yield 0.01M |
| formulation, pH) | PBS; pH 7.4 |
| Storage conditions | Room temperature |

Reference Item

TABLE 8
Material table - Reference item

| Material | Reference item |
|---|---|
| Denomination | Alphagan ® (brimonidine) |
| Concentration | 2 mg/mL |
| Characteristics (form, aspect, | Solution |
| molecular weight, etc.) | |
| Supplier | Allergan |
| Batch number | E72453 |
| Purity | NA |
| Preparation | Ready to use |
| Storage conditions | Room temperature |

Animals and Husbandry

All animals were treated according to the Directive 2010/63/UE European Convention for the Protection of Vertebrate Animals used for Experimental and other scientific purposes, and to the Association for Research in Vision and Ophthalmology (ARVO) Statement for the Use of Animals in Ophthalmic and Vision Research.

Animals

Animals

Species: Rat. This is the species most commonly used in this experimental model.

Strain: Sprague Dawley (albino).

Age: Approximately 6 weeks (on ordering).

Number/sex: 68 males (study 60: reserve 8).

Breeder: “Charles River”—F-69592 L'Arbresie Cedex.

Identification

Tails were marked using a permanent ink marker, following the inclusion examination.

Clinical Examination and Health Status

Animals were held in observation for at least 1 week following their arrival. Animals were observed daily for signs of illness and particular attention was paid to their eyes.

Housing

Animal Husbandry

Animals were housed in standard cages (two or three animals per cage), under identical environmental conditions. The temperature was held at 22±2° C. and the relative humidity at 55±10%. Rooms were continuously ventilated (15 air volumes per hour).

Temperature and relative humidity were continuously controlled and recorded. Animals were routinely exposed (in-cage) to 10-200 lx light in a 12-hour light and darkness cycle.

Food and Water

Throughout the study, animals had free access to food and water. They were fed a standard dry pellet diet (Rod16-H-LASvendi GmbH D-59494 Soest Germany). Tap water, regularly analyzed, was available ad libitum from plastic bottles.

Design and Procedure

Study Design

The schedule is presented in Table 9.

TABLE 9
Study design

| | | | | | | Endpoints | |
|---|---|---|---|---|---|---|---|
| Group | Rat | | Treatment | | | | Histological |
| #ID | #ID | Induction | Compound | Dose | Dosing regimen | ERG | evaluation |
| 1 | R#13 | Retinal | MANF | 0.15 | Intravitreal | Scotopic | RGCs |
| | to | ischemia | | mg/mL | administration in | conditions | quantification |
| | R#24 | by optic | | | right eyes (4 μL) | 0 dB, both | on |
| 2 | R#37 | nerve | MANF | 0.5 | once, | eyes | flatmounted |
| | to | clamping | | mg/mL | immediately after | Baseline | injured |
| | R#48 | for 45 | | | reperfusion | and Day 7 | retinas on |
| 3 | R#25 | min on | MANF | 1.5 | | | Day 7 |
| | to | right eye | | mg/mL | | | |
| | R#36 | | | | | | |
| 4 | R#1 | | Vehicle | — | | | |
| | to | | (PBS) | | | | |
| | R#12 | | | | | | |
| 5 | R#49 | | Alphagan ® | 1 | Intraperitoneal | | |
| | to | | (Brimonidine) | mg/kg | administration | | |
| | R#60 | | | | (0.5 mL/kg) once | | |
| | | | | | 30 min before | | |
| | | | | | induction | | |

Experimental Procedure

Selection of the Animals

Sixty (60) animals were included in this study out of sixty-eight (68) ordered.

Only healthy animals with no visible sign of ocular defect were randomly assigned to the study groups, using a macrofunction in Excel® software on the basis of ERG baseline (b-wave amplitude in right eye).

Route and Method of Administration

Test and control items were injected intravitreally (4 μL) in right eye from anesthetized rat using a mix of xylazine/ketamine and a 30-G needle mounted on a syringe, immediately after reperfusion.

Reference item was intraperitoneally dosed using a 0.5 mL/kg volume of administration, 30 min before induction.

General In Vivo Observations

Body Weight

The body weight of all animals was recorded before the start of the study, on Day 0 and at the end of the study (Day 7).

General Behavior

Each day, the general behavior and the aspect of all animals were observed.

Ischemia/Reperfusion Methods and Measurements

Clamping

Right eyes underwent a temporal orbitectomy combined with periorbital stripping. The globe remained in the orbit and was completely isolated on a pedicle consisting of the optic nerve, ophthamociliary arteries and the venous outflow. A clamp placed around the pedicle initiated the global ocular ischemia when tightened.

Ischemia was maintained for 45 minutes. The reperfusion period was initiated by the release of the clamp.

Evaluations and Measurements

ERG was recorded before ischemia (baseline) and 7 days after reperfusion in both eyes. The a-wave and b-wave amplitudes (μV) were measured for each ERG; the a-wave and b-wave amplitudes as a percentage of the baseline values obtained before ischemia. Fifteen (15) min before measurement, 10 μL. Mydriaticum (0.5% tropicamide) were instilled for pupillary dilatation.

ERG parameters:
Color: white maximum.
Maximum intensity: 2.6 cd·s/m2 (0 dB); Duration 0.24 ms; number of flash: 1.
Filter: 50 Hz.
Impedance Threshold: 90 kΩ.

Study Termination and Retinal Ganglion Cell (RGC) Evaluation

At the end of the study, animals were euthanized by intraperitoneal injection of overdosed pentobarbital. This method is one of the recommended methods for euthanasia by European authorities.

After euthanasia, the right eyeballs were fixed in Formalin 4% (24 h at 4° C.), dissected and retinas were flat mounted. Two left eyes retinas per group were sampled and processed the same as the right eye retinas. They served as naïve controls. The flat mounted preparations were stained with an Alexa 594 conjugated anti-BRN3A (Brain-specific hoeobox/POU domain protein 3A, Chemicon, cat #mAb1585) to label RGC. Fluorescence images were recorded with Apotome microscope at magnification ×10 (Zeiss). RGC were counted with Image J software in 8 locations for each retina area (2 pictures per quarter). The cell count was reported in cell/mm².

Data Processing

Results were expressed in individual and summarized data tables using Microsoft Excel® Software.

Statistical Analysis

The statistical analyses were performed using the software GraphPad Prism.

The statistical analysis results are summarized in Tables 10 and 11.

a- and b-wave amplitudes were expressed as mean and standard deviation.

A Kruskal-Wallis analysis was performed on the individual right eye b-wave amplitudes. The drug effect was assessed using the Dunn's multiple comparison tests; each treated group was compared to the vehicle.

A Kruskal-Wallis analysis was performed on the individual RGC densities. The drug effect was assessed using the Dunn's multiple comparison tests; each treated group was compared to the vehicle,

The p value has to be lower than 0.05 for the difference to be significant.

Results

General Behavior and Body Weight

Body weights measures are reported in Table 12.

All animals showed a normal body weight gain from baseline to Day 7.

No abnormal behaviour or unhealthy signs were found for any treated animals during the study period. However, 3 animals died during the study:
Animal R#55 treated with 1 mg/kg brimonidine was found dead on Day 0 after clamping.
Animal R#56 treated with 1 mg/kg brimonidine and animal treated with PBS were found dead on Day 7 before evaluations.

These deaths were related to anesthesia and not to the treatment.

TABLE 10
Statistical analysis - Right eye b-wave amplitudes on Day 7
(% baseline)

| | |
|---|---|
| Kruskal-Wallis test P value | 0.0243 |
| Exact or approximate P value? | Approximate |
| P value summary | * |
| Do the medians vary signif. (P < 0.05) | Yes |
| Number of groups | 5 |
| Kruskal-Wallis statistic | 11.21 |
| Data summary | |
| Number of treatments (columns) | 5 |
| Number of values (total) | 56 |
| Number of families | 1 |
| Number of comparisons per family | 4 |
| Alpha | 0.05 |

| Dunn's multiple | Mean rank | | | Adjusted |
|---|---|---|---|---|
| comparisons test | diff. | Significant? | Summary | P Value |
| PBS vs. MANF | −14.09 | No | ns | 0.1712 |
| 0.15 mg/mL | | | | |
| PBS vs. MANF | −19.2 | Yes | * | 0.0193 |
| 0.5 mg/mL | | | | |
| PBS vs. MANF | −10.7 | No | ns | 0.4648 |
| 1.5 mg/mL | | | | |
| PBS vs. Aphagan | −20.66 | Yes | * | 0.015 |

| | Mean | Mean | Mean | | |
|---|---|---|---|---|---|
| Test details | rank 1 | rank 2 | rank diff. | n1 | n2 |
| PBS vs. MANF | 15.64 | 29.73 | −14.09 | 11 | 11 |
| 0.15 mg/mL | | | | | |
| PBS vs. MANF | 15.64 | 34.83 | −19.2 | 11 | 12 |
| 0.5 mg/mL | | | | | |
| PBS vs. MANF | 15.64 | 26.33 | −10.7 | 11 | 12 |
| 1.5 mg/mL | | | | | |
| PBS vs. Aphagan | 15.64 | 36.3 | −20.66 | 11 | 10 |

TABLE 11
Statistical analysis - Surviving retinal ganglion cell
density or Day 7

| | |
|---|---|
| Kruskal-Wallis test P value | 0.0667 |
| Exact or approximate P value? | Approximate |
| P value summary | ns |
| Do the medians vary signif. (P < 0.05) | No |
| Number of groups | 5 |
| Kruskal-Wallis statistic | 8.78 |
| Data summary | |
| Number of treatments (columns) | 5 |
| Number of values (total) | 58 |
| Number of families | 1 |
| Number of comparisons per family | 4 |
| Alpha | 0.05 |

| Dunn's multiple | Mean rank | | | Adjusted |
|---|---|---|---|---|
| comparisons test | diff. | Significant? | Summary | P Value |
| PBS vs. MANF | −8.48 | No | ns | 0.915 |
| 0.15 mg/mL | | | | |
| PBS vs. MANF | −11 | No | ns | 0.4712 |
| 0.5 mg/mL | | | | |
| PBS vs. MANF | −14 | No | ns | 0.1892 |
| 1.5 mg/mL | | | | |
| PBS vs. Aphagan | −20.5 | Yes | * | 0.0177 |

| | Mean | Mean | Mean | | |
|---|---|---|---|---|---|
| Test details | rank 1 | rank 2 | rank diff. | n1 | n2 |
| PBS vs. MANF | 18.7 | 27.2 | −8.48 | 11 | 12 |
| 0.15 mg/mL | | | | | |
| PBS vs. MANF | 18.7 | 29.7 | −11 | 11 | 12 |
| 0.5 mg/mL | | | | | |
| PBS vs. MANF | 18.7 | 32.7 | −14 | 11 | 12 |
| 1.5 mg/mL | | | | | |
| PBS vs. Aphagan | 18.7 | 39.2 | −20.5 | 11 | 11 |

TABLE 12
Animal body weight
Body weights (g)

| | Rat | | | | Day | | | Sacrifice | | |
|---|---|---|---|---|---|---|---|---|---|---|
| Treatment | id | Baseline | Mean | SD | 0 | Mean | SD | day | Mean | SD |
| MANF | 13 | 228 | 228 | 4 | 316 | 322 | 15 | 343 | 343 | 20 |
| 0.15 | 14 | 224 | | | 324 | | | 349 | | |
| mg/mL | 15 | 226 | | | 321 | | | 335 | | |
| | 16 | 224 | | | 304 | | | 316 | | |
| | 17 | 236 | | | 323 | | | 336 | | |
| | 18 | 230 | | | 335 | | | 358 | | |
| | 19 | 226 | | | 340 | | | 371 | | |
| | 20 | 236 | | | 343 | | | 370 | | |
| | 21 | 222 | | | 294 | | | 309 | | |
| | 22 | 232 | | | 317 | | | 344 | | |
| | 23 | 228 | | | 335 | | | 362 | | |
| | 24 | 228 | | | 312 | | | 326 | | |
| MANF | 37 | 230 | 226 | 7 | 300 | 323 | 15 | 316 | 345 | 18 |
| 0.5 | 38 | 234 | | | 333 | | | 357 | | |
| mg/mL | 39 | 224 | | | 302 | | | 323 | | |
| | 40 | 226 | | | 301 | | | 312 | | |
| | 41 | 232 | | | 343 | | | 364 | | |
| | 42 | 210 | | | 317 | | | 356 | | |
| | 43 | 224 | | | 315 | | | 347 | | |
| | 44 | 234 | | | 330 | | | 355 | | |
| | 45 | 224 | | | 339 | | | 352 | | |
| | 46 | 222 | | | 323 | | | 340 | | |
| | 47 | 234 | | | 338 | | | 357 | | |
| | 48 | 218 | | | 330 | | | 360 | | |
| MANF | 25 | 228 | 229 | 10 | 321 | 328 | 24 | 345 | 347 | 30 |
| 1.5 | 26 | 232 | | | 329 | | | 341 | | |
| mg/mL | 27 | 218 | | | 280 | | | 285 | | |
| | 28 | 224 | | | 331 | | | 352 | | |
| | 29 | 238 | | | 344 | | | 375 | | |
| | 30 | 220 | | | 327 | | | 348 | | |
| | 31 | 218 | | | 326 | | | 346 | | |
| | 32 | 240 | | | 369 | | | 390 | | |
| | 33 | 244 | | | 359 | | | 386 | | |
| | 34 | 216 | | | 297 | | | 307 | | |
| | 35 | 242 | | | 333 | | | 347 | | |
| | 36 | 228 | | | 325 | | | 336 | | |
| PBS | 1 | 222 | 226 | 6 | 321 | 305 | 88 | 344 | 350 | 26 |
| | 2 | 230 | | | 335 | | | 344 | | |
| | 3 | 238 | | | 360 | | | 378 | | |
| | 4 | 222 | | | 293 | | | 311 | | |
| | 5 | 216 | | | 319 | | | 345 | | |
| | 6 | 220 | | | 295 | | | 296 | | |
| | 7 | 220 | | | 329 | | | 350 | | |
| | 8 | 228 | | | 333 | | | 360 | | |
| | 9 | 230 | | | 362 | | | 393 | | |
| | 10 | 228 | | | 342 | | | 362 | | |
| | 11 | 232 | | | 340 | | | 363 | | |
| | 12 | 226 | | | 33 | | | 358 | | |
| Alphagan | 49 | 226 | 228 | 5 | 321 | 328 | 14 | 334 | 345 | 28 |
| 2 mg/mL | 50 | 228 | | | 341 | | | 370 | | |
| | 51 | 232 | | | 322 | | | 350 | | |
| | 52 | 228 | | | 343 | | | 362 | | |
| | 53 | 228 | | | 326 | | | 342 | | |
| | 54 | 232 | | | 328 | | | 334 | | |
| | 55 | 232 | | | 330 | | | NA | | |
| | 56 | 230 | | | 318 | | | 339 | | |
| | 57 | 232 | | | 350 | | | 387 | | |
| | 58 | 218 | | | 320 | | | 328 | | |
| | 59 | 232 | | | 340 | | | 363 | | |
| | 60 | 220 | | | 301 | | | 282 | | |

Electroretinogramns (ERG)

The functional status of the retina was evaluated by electroretinography one week after the ischemic insult.

The b-wave is induced by potassium efflux shunted from activated bipolar cells by Miller cells and is an electrophysiological indicator of retinal signal transmission. The a-wave, which is induced by light-activated hyperpolarization of photoreceptors, is usually less affected by changes in blood flow.

Individual data, summarized in Table 13, are reported in Tables 14-18.

A marked but not significant protection from the reduction in b-wave amplitude was observed after intravitreal administration of MANF at 0.15 mg/mL and 1.5 mg/mL, in comparison with the PBS-treated group. The b-wave amplitudes recovered by 49% and 47% from the mean baseline value for the groups treated with MANF at 0.15 mg/mL and 1.5 ng/mL, respectively, while PBS-treated group displayed a 37% recovery.

A significant improvement of the b-wave recovery was shown after intravitreal administration of MANF at 0.5 mg/mL (p=0.0193). The b-wave amplitude recovered by 52% of the baseline mean value.

Prophylactically administered Alphagan® (1 mg/kg brimonidine) led to a significant protection of the b-wave amplitude in comparison with the vehicle group (p=0.015). The b-wave amplitude recovered by 59% of the baseline mean value.

TABLE 13
Right eye ERG measurements

B-wave in right eye

| Treatment | Time-point | amplitude (μV) | amplitude (%) |
|---|---|---|---|
| MANF | Baseline | 1071.8 | — |
| 0.15 mg/mL | Day 7 | 523.0 | 49.0 |
| MANF | Baseline | 1050.3 | — |
| 0.5 mg/mL | Day 7 | 539.9 | 52.1 |
| MANF | Baseline | 1071.7 | — |
| 1.5 mg/mL | Day 7 | 492.8 | 46.8 |
| PBS | Baseline | 1052.8 | — |
| | Day 7 | 387.7 | 37.4 |
| Alphagan ® | Baseline | 1077.4 | — |
| 2 mg/mL | Day 7 | 607.4 | 58.6 |

TABLE 14
ERG measurements in both eyes with MANF 0.15 mg/mL; a = aberrant

| | | Amplitude (μV) | | | | Amplitude (% baseline) | |
|---|---|---|---|---|---|---|---|
| | | Baseline | | Day 7 | | Day 7 | |
| Rat id | eye | A-wave | B-wave | A-wave | B-wave | A-wave | B-wave |
| 13 | treated | −380 | 929 | −154 | 448 | 41 | 48 |
| | right | | | | | | |
| | control left | −423 | 785 | −330 | 922 | 78 | 117 |
| 14 | treated | −410 | 846 | −263 | 598 | 64 | 71 |
| | right | | | | | | |
| | control left | −434 | 818 | −383 | 986 | 88 | 121 |
| 15 | treated | −508 | 898 | −264 | 566 | 52 | 63 |
| | right | | | | | | |
| | control left | −498 | 886 | −493 | 1170 | 99 | 132 |
| 16 | treated | −434 | 989 | −283 | 557 | 65 | 56 |
| | right | | | | | | |
| | control left | −398 | 887 | −404 | 914 | 102 | 103 |
| 17 | treated | −457 | 998 | −201 | 463 | 44 | 46 |
| | right | | | | | | |
| | control left | −454 | 972 | −412 | 939 | 91 | 97 |
| 18 | treated | −494 | 961 | −283 | 0^{a} | 57 | — |
| | right | | | | | | |
| | control left | −462 | 877 | −243 | 380^{a} | 53 | — |
| 19 | treated | −503 | 1120 | −152 | 425 | 30 | 38 |
| | right | | | | | | |
| | control left | −467 | 1010 | −383 | 812 | 82 | 80 |
| 20 | treated | −497 | 1120 | −235 | 494 | 47 | 44 |
| | right | | | | | | |
| | control left | −502 | 1070 | −343 | 850 | 68 | 79 |
| 21 | treated | −480 | 1090 | −325 | 295 | 68 | 27 |
| | right | | | | | | |
| | control left | −526 | 996 | −459 | 562 | 87 | 56 |
| 22 | treated | −627 | 1410 | −407 | 860 | 65 | 61 |
| | right | | | | | | |
| | control left | −613 | 1220 | −475 | 1100 | 77 | 90 |
| 23 | treated | −476 | 1320 | −255 | 557 | 54 | 42 |
| | right | | | | | | |
| | control left | −445 | 1250 | −405 | 1070 | 91 | 86 |
| 24 | treated | −496 | 1180 | −308 | 490 | 62 | 42 |
| | right | | | | | | |
| | control left | −550 | 1280 | −405 | 897 | 74 | 70 |
| Mean | treated | −480.2 | 1071.8 | −260.8 | 523.0 | 54.1 | 49.0 |
| SD | right | 61.2 | 170.2 | 71.5 | 140.0 | 11.7 | 12.7 |
| SEM | | 17.7 | 49.1 | 20.6 | 42.2 | 3.4 | 3.8 |
| Mean | control left | −481.0 | 1004.3 | −394.6 | 929.3 | 82.5 | 93.8 |
| SD | | 60.3 | 169.3 | 68.1 | 163.2 | 13.6 | 22.9 |
| SEM | | 17.4 | 48.9 | 19.7 | 49.2 | 3.9 | 6.9 |

TABLE 15
ERG measurements in both eyes with MANF 0.5 mg/mL; a = aberrant

| | | Amplitude (μV) | | | | Amplitude (% baseline) | |
|---|---|---|---|---|---|---|---|
| | | Baseline | | Day 7 | | Day 7 | |
| Rat id | eye | A-wave | B-wave | A-wave | B-wave | A-wave | B-wave |
| 37 | treated | −452 | 899 | −247 | 561 | 55 | 62 |
| | right | | | | | | |
| | control left | −416 | 843 | −442 | 933 | 106 | 111 |
| 38 | treated | −399 | 869 | −260 | 462 | 65 | 53 |
| | right | | | | | | |
| | control left | −428 | 943 | −405 | 1020 | 95 | 108 |
| 39 | treated | −467 | 957 | −262 | 577 | 56 | 60 |
| | right | | | | | | |
| | control left | −506 | 1000 | −414 | 951 | 82 | 95 |
| 40 | treated | −472 | 986 | −306 | 700 | 65 | 71 |
| | right | | | | | | |
| | control left | −460 | 979 | −409 | 1090 | 89 | 111 |
| 41 | treated | −487 | 965 | −237 | 557 | 49 | 58 |
| | right | | | | | | |
| | control left | −439 | 855 | −388 | 934 | 88 | 109 |
| 42 | treated | −453 | 997 | −293 | 563 | 65 | 56 |
| | right | | | | | | |
| | control left | −482 | 1060 | −480 | 1130 | 100 | 107 |
| 43 | treated | −565 | 1050 | −225 | 494 | 40 | 47 |
| | right | | | | | | |
| | control left | −468 | 859 | −353 | 865 | 75 | 101 |
| 44 | treated | −545 | 1140 | −319 | 545 | 59 | 48 |
| | right | | | | | | |
| | control left | −489 | 1040 | −434 | 925 | 89 | 89 |
| 45 | treated | −537 | 1160 | −264 | 417 | 49 | 36 |
| | right | | | | | | |
| | control left | −456 | 965 | −383 | 844 | 84 | 87 |
| 46 | treated | −445 | 1180 | −392 | 782 | 88 | 66 |
| | right | | | | | | |
| | control left | −403 | 1100 | −404 | 1010 | 100 | 92 |
| 47 | treated | −596 | 1230 | −329 | 662 | 55 | 54 |
| | right | | | | | | |
| | control left | −519 | 1060 | −460 | 936 | 89 | 88 |
| 48 | treated | −475 | 1170 | −93 | 159 | 20 | 14 |
| | right | | | | | | |
| | control left | −443 | 1090 | −329 | 717 | 74 | 66 |
| Mean | treated | −491.1 | 1050.3 | −268.9 | 539.9 | 55.4 | 52.1 |
| SD | right | 57.4 | 121.4 | 72.4 | 156.7 | 16.4 | 15.3 |
| SEM | | 16.6 | 35.1 | 20.9 | 45.2 | 4.7 | 4.4 |
| Mean | control left | −459.1 | 982.8 | −408.4 | 946.3 | 89.2 | 97.0 |
| SD | | 35.6 | 92.4 | 42.7 | 110.6 | 9.7 | 13.5 |
| SEM | | 10.3 | 26.7 | 12.3 | 31.9 | 2.8 | 3.9 |

TABLE 16
ERG measurements in both eyes with MANF 1.5 mg/mL; ; a = aberrant

| | | Amplitude (μV) | | | | Amplitude (% baseline) | |
|---|---|---|---|---|---|---|---|
| | | Baseline | | Day 7 | | Day 7 | |
| Rat id | eye | A-wave | B-wave | A-wave | B-wave | A-wave | B-wave |
| 25 | treated | −320 | 841 | −160 | 482 | 50 | 57 |
| | right | | | | | | |
| | control left | −283 | 744 | −262 | 921 | 93 | 124 |
| 26 | treated | −398 | 838 | −225 | 270 | 57 | 32 |
| | right | | | | | | |
| | control left | −439 | 885 | −411 | 970 | 94 | 110 |
| 27 | treated | −359 | 831 | −269 | 563 | 75 | 68 |
| | right | | | | | | |
| | control left | −396 | 944 | −405 | 1130 | 102 | 120 |
| 28 | treated | −447 | 1010 | −242 | 442 | 54 | 44 |
| | right | | | | | | |
| | control left | −490 | 1030 | −376 | 808 | 77 | 78 |
| 29 | treated | −419 | 960 | −254 | 556 | 61 | 58 |
| | right | | | | | | |
| | control left | −387 | 917 | −380 | 985 | 98 | 107 |
| 30 | treated | −494 | 1030 | −156 | 426 | 32 | 41 |
| | right | | | | | | |
| | control left | −467 | 973 | −399 | 1040 | 85 | 107 |
| 31 | treated | −487 | 1110 | −296 | 590 | 61 | 53 |
| | right | | | | | | |
| | control left | −464 | 1040 | −391 | 939 | 84 | 90 |
| 32 | treated | −459 | 1120 | −299 | 417 | 65 | 37 |
| | right | | | | | | |
| | control left | −399 | 915 | −414 | 810 | 104 | 89 |
| 33 | treated | −481 | 1060 | −255 | 456 | 53 | 43 |
| | right | | | | | | |
| | control left | −417 | 895 | −393 | 942 | 94 | 105 |
| 34 | treated | −740 | 1550 | −262 | 579 | 35 | 37 |
| | right | | | | | | |
| | control left | −677 | 1440 | −459 | 1080 | 68 | 75 |
| 35 | treated | −539 | 1230 | −273 | 588 | 51 | 48 |
| | right | | | | | | |
| | control left | −537 | 1060 | −433 | 805 | 81 | 76 |
| 36 | treated | −541 | 1280 | −258 | 544 | 48 | 43 |
| | right | | | | | | |
| | control left | −416 | 1050 | −409 | 1010 | 98 | 96 |
| Mean | treated | −473.7 | 1071.7 | −245.8 | 492.8 | 53.4 | 46.8 |
| SD | right | 107.4 | 210.0 | 45.8 | 96.0 | 11.9 | 10.4 |
| SEM | | 31.0 | 60.6 | 13.2 | 27.7 | 3.4 | 3.0 |
| Mean | control left | −447.7 | 991.1 | −394.3 | 953.3 | 89.8 | 98.1 |
| SD | | 95.8 | 167.6 | 47.4 | 106.3 | 11.0 | 16.6 |
| SEM | | 27.6 | 48.4 | 13.7 | 30.7 | 3.2 | 4.8 |

TABLE 17
ERG measurements in both eyes with PBS; † = anesthesia death.

| | | Amplitude (μV) | | | | Amplitude (% baseline) | |
|---|---|---|---|---|---|---|---|
| | | Baseline | | Day 7 | | Day 7 | |
| Rat id | eye | A-wave | B-wave | A-wave | B-wave | A-wave | B-wave |
| 1 | treated | −354 | 786 | −117 | 260 | 33 | 33 |
| | right | | | | | | |
| | control left | −378 | 844 | −438 | 1010 | 116 | 120 |
| 2 | treated | −372 | 831 | −219 | 312 | 59 | 38 |
| | right | | | | | | |
| | control left | −396 | 893 | −317 | 804 | 80 | 90 |
| 3 | treated | −475 | 808 | −176 | 297 | 37 | 37 |
| | right | | | | | | |
| | control left | −499 | 876 | −348 | 652 | 70 | 74 |
| 4 | treated | −395 | 968 | −233 | 562 | 59 | 58 |
| | right | | | | | | |
| | control left | −432 | 984 | −398 | 1070 | 92 | 109 |
| 5 | treated | −590 | 1040 | −271 | 514 | 46 | 49 |
| | right | | | | | | |
| | control left | −512 | 907 | −481 | 950 | 94 | 105 |
| 6 | treated | −556 | 1050 | −210 | 324 | 38 | 31 |
| | right | | | | | | |
| | control left | −574 | 1080 | −357 | 704 | 62 | 65 |
| 7 | treated | −503 | 1100 | † | † | † | † |
| | right | | | | | | |
| | control left | −434 | 947 | † | † | † | † |
| 8 | treated | −456 | 1060 | −277 | 502 | 61 | 47 |
| | right | | | | | | |
| | control left | −444 | 1030 | −496 | 1100 | 112 | 107 |
| 9 | treated | −442 | 1070 | −153 | 302 | 35 | 28 |
| | right | | | | | | |
| | control left | −466 | 1070 | −332 | 793 | 71 | 74 |
| 10 | treated | −535 | 1280 | −196 | 492 | 37 | 38 |
| | right | | | | | | |
| | control left | −534 | 1080 | −387 | 924 | 72 | 86 |
| 11 | treated | −481 | 1220 | −74 | 222 | 15 | 18 |
| | right | | | | | | |
| | control left | −447 | 987 | −419 | 1160 | 94 | 118 |
| 12 | treated | −618 | 1420 | −322 | 478 | 52 | 34 |
| | right | | | | | | |
| | control left | −550 | 1240 | −361 | 910 | 66 | 73 |
| Mean | treated | −481.4 | 1052.8 | −204.4 | 387.7 | 42.8 | 37.4 |
| SD | right | 83.8 | 191.8 | 72.6 | 121.5 | 14.0 | 11.0 |
| SEM | | 24.2 | 55.4 | 21.9 | 36.6 | 4.2 | 3.3 |
| Mean | control left | −472.2 | 994.8 | −394.0 | 916.1 | 84.4 | 92.7 |
| SD | | 61.6 | 112.4 | 59.1 | 164.2 | 18.4 | 19.5 |
| SEM | | 17.8 | 32.4 | 17.8 | 49.5 | 5.5 | 5.9 |

TABLE 18
ERG measurements in both eyes wth Alphagan; † = anasthesia death.

| | | Amplitude (μV) | | | | Amplitude (% baseline) | |
|---|---|---|---|---|---|---|---|
| | | Baseline | | Day 7 | | Day 7 | |
| Rat id | eye | A-wave | B-wave | A-wave | B-wave | A-wave | B-wave |
| 49 | treated | −399 | 905 | −307 | 616 | 77 | 68 |
| | right | | | | | | |
| | control left | −328 | 843 | −346 | 938 | 105 | 111 |
| 50 | treated | −491 | 896 | −330 | 370 | 67 | 41 |
| | right | | | | | | |
| | control left | −530 | 979 | −493 | 910 | 93 | 93 |
| 51 | treated | −461 | 808 | −390 | 894 | 85 | 111 |
| | right | | | | | | |
| | control left | −424 | 885 | −415 | 1010 | 98 | 114 |
| 52 | treated | −416 | 1040 | −309 | 700 | 74 | 67 |
| | right | | | | | | |
| | control left | −404 | 973 | −365 | 928 | 90 | 95 |
| 53 | treated | −450 | 1020 | −177 | 516 | 39 | 51 |
| | right | | | | | | |
| | control left | −429 | 1010 | −328 | 1010 | 76 | 100 |
| 54 | treated | −447 | 1030 | −262 | 462 | 59 | 45 |
| | right | | | | | | |
| | control left | −484 | 961 | −405 | 884 | 84 | 92 |
| 55 | treated | −510 | 1160 | † | † | † | † |
| | right | | | | | | |
| | control left | −424 | 949 | † | † | † | † |
| 56 | treated | −555 | 1050 | † | † | † | † |
| | right | | | | | | |
| | control left | −552 | 1130 | † | † | † | † |
| 57 | treated | −532 | 1150 | −309 | 669 | 58 | 58 |
| | right | | | | | | |
| | control left | −567 | 1210 | −504 | 1080 | 89 | 89 |
| 58 | treated | −527 | 1300 | −310 | 565 | 59 | 43 |
| | right | | | | | | |
| | control left | −520 | 1120 | −386 | 845 | 74 | 75 |
| 59 | treated | −558 | 1200 | −392 | 812 | 70 | 68 |
| | right | | | | | | |
| | control left | −577 | 1222 | −445 | 1040 | 77 | 85 |
| 60 | treated | −538 | 1370 | −307 | 470 | 57 | 34 |
| | right | | | | | | |
| | control left | −484 | 1200 | −366 | 875 | 76 | 73 |
| Mean | treated | −490.3 | 1077.4 | −309.3 | 607.4 | 64.5 | 58.6 |
| SD | right | 54.5 | 166.6 | 61.0 | 164.3 | 12.9 | 21.9 |
| SEM | | 15.7 | 48.1 | 19.3 | 52.0 | 4.1 | 6.9 |
| Mean | control left | −476.9 | 1040.2 | −405.3 | 952.0 | 86.3 | 92.8 |
| SD | | 76.2 | 130.9 | 59.8 | 78.5 | 10.6 | 13.4 |
| SEM | | 22.0 | 37.8 | 18.9 | 24.8 | 3.4 | 4.2 |

Retinal Ganglion Cell Survival

To assess the effect of the treatment on RGC viability, RGC density was evaluated 7 days after ischemia. Individual data, summarized in Table 19, are reported in table 20 and 21.

The RGC density in the retina of non-ischemic eyes (two left eyes per group) was 2121±45-RGC/mm² (n=12).

One week after ischemia, mean RGC density decreased to 264±261 RGC/mm² (−88′% compared to non-ischemic eyes) in the PBS-treated group.

Intravitreal administration of MANF at 0.15 mg/mL, 0.5 mg/mL and 1.5 mg/mL, showed a trend in improvement of the mean RGC survival 7 days after injury with 403±189 cells/mm², 465±301 cells/mm² and 488±14 cells/mm², respectively.

Intraperitoneal administration of Alphagan® (1 mg/kg brimonidine) significantly prevented from the decrease of surviving RGCs, with 578±185 RGCs/mm² (p=0.0177), when compared with the PBS-treated group.

TABLE 19
Surviving retinal ganglion cell density in both eyes

| | | Alive cells (BrN3A | Alive cells (BrN3A | |
|---|---|---|---|---|
| | | positive) (cells/mm²) | positive) (cells/mm²) in | |
| | | in right eye | left eye (n = 2/group) | |
| Treatment | | (n = 12/group) | Mean | SD |
| MANF | Mean | 402.8 | 2 120.5 | 454.5 |
| 0.15 mg/mL | SD | 189.2 | | |
| MANF | Mean | 464.9 | | |
| 0.5 mg/mL | SD | 301.3 | | |
| MANF | Mean | 487.7 | | |
| 1.5 mg/mL | SD | 213.8 | | |
| PBS | Mean | 264.3 | | |
| | SD | 261.2 | | |
| Alphagan ® | Mean | 578.0 | | |
| 2 mg/mL | SD | 185.4 | | |

TABLE 20
Right injured eye survival RGCs

| | | Alive cells (BrN3A positive) in right eye | | | | | | | | |
|---|---|---|---|---|---|---|---|---|---|---|
| | | cells/photo | | | | | | | | individual |
| | rat | Value | Value | Value | Value | Value | Value | Value | Value | mean |
| Treatment | id | 1 | 2 | 3 | 4 | 5 | 6 | 7 | 8 | cells/mm² |
| MANF | 13 | 11 | 24 | 39 | 26 | 5 | 12 | 31 | 78 | 47 |
| 0.15 | 14 | 389 | 162 | 6 | 9 | 240 | 17 | 42 | 143 | 210 |
| mg/mL | 15 | 503 | 377 | 284 | 377 | 228 | 234 | 133 | 115 | 469 |
| | 16 | 446 | 489 | 484 | 342 | 321 | 322 | 728 | 332 | 722 |
| | 17 | 391 | 444 | 484 | 560 | 220 | 336 | 263 | 356 | 636 |
| | 18 | 305 | 305 | 227 | 311 | 272 | 233 | 345 | 261 | 471 |
| | 19 | 235 | 143 | 261 | 312 | 324 | 500 | 233 | 180 | 456 |
| | 20 | 221 | 221 | 250 | 356 | 247 | 7 | 321 | 171 | 374 |
| | 21 | 228 | 18 | 194 | 39 | 205 | 79 | 5 | 7 | 161 |
| | 22 | 404 | 409 | 348 | 7 | 235 | 430 | 123 | 116 | 432 |
| | 23 | 247 | 293 | 260 | 198 | 211 | 260 | 268 | 223 | 408 |
| | 24 | 266 | 326 | 125 | 78 | 318 | 365 | 295 | 379 | 448 |
| | Mean | | | | | | | | | 402.8 |
| | SD | | | | | | | | | 189.2 |
| MANF | 37 | 227 | 220 | 332 | 387 | 372 | 359 | 335 | 224 | 512 |
| 0.5 | 38 | 248 | 264 | 309 | 244 | 335 | 197 | 248 | 279 | 443 |
| mg/mL | 39 | 570 | 400 | 456 | 240 | 244 | 292 | 392 | 348 | 613 |
| | 40 | 165 | 138 | 278 | 144 | 183 | 304 | 295 | 241 | 364 |
| | 41 | 290 | 325 | 278 | 190 | 199 | 208 | 142 | 188 | 379 |
| | 42 | 356 | 284 | 353 | 267 | 349 | 478 | 279 | 270 | 549 |
| | 43 | 255 | 200 | 256 | 297 | 124 | 121 | 241 | 117 | 336 |
| | 44 | 105 | 0 | 55 | 233 | 1 | 1 | 62 | 10 | 97 |
| | 45 | 0 | 151 | 103 | 357 | 121 | 6 | 219 | 238 | 249 |
| | 46 | 782 | 517 | 765 | 722 | 786 | 753 | 719 | 761 | 1209 |
| | 47 | 6 | 0 | 2 | 7 | 100 | 84 | 111 | 194 | 105 |
| | 48 | 585 | 554 | 425 | 254 | 395 | 443 | 426 | 391 | 724 |
| | Mean | | | | | | | | | 464.9 |
| | SD | | | | | | | | | 301.3 |
| MANF | 25 | 370 | 375 | 308 | 325 | 250 | 370 | 324 | 299 | 546 |
| 1.5 | 26 | 287 | 119 | 210 | 135 | 228 | 323 | 275 | 239 | 378 |
| mg/mL | 27 | 421 | 308 | 346 | 196 | 321 | 299 | 418 | 328 | 549 |
| | 28 | 451 | 468 | 497 | 438 | 181 | 170 | 618 | 645 | 723 |
| | 29 | 398 | 359 | 397 | 405 | 320 | 152 | 381 | 160 | 536 |
| | 30 | 511 | 468 | 509 | 486 | 440 | 618 | 912 | 730 | 974 |
| | 31 | 113 | 148 | 188 | 126 | 107 | 90 | 23 | 151 | 197 |
| | 32 | 188 | 373 | 152 | 469 | 372 | 366 | 331 | 356 | 543 |
| | 33 | 302 | 300 | 348 | 340 | 354 | 290 | 304 | 102 | 488 |
| | 34 | 183 | 191 | 104 | 83 | 122 | 130 | 200 | 236 | 260 |
| | 35 | 28 | 27 | 37 | 393 | 228 | 386 | 420 | 124 | 342 |
| | 36 | 160 | 175 | 253 | 128 | 162 | 197 | 121 | 320 | 316 |
| | Mean | | | | | | | | | 487.7 |
| | SD | | | | | | | | | 213.8 |
| PBS | 1 | 299 | 203 | 532 | 409 | 302 | 157 | 161 | 247 | 481 |
| | 2 | 52 | 5 | 37 | 14 | 62 | 54 | 65 | 27 | 66 |
| | 3 | 4 | 6 | 8 | 133 | 66 | 89 | 12 | 40 | 75 |
| | 4 | 38 | 99 | 44 | 64 | 7 | 61 | 11 | 10 | 70 |
| | 5 | 50 | 24 | 75 | 55 | 113 | 215 | 184 | 243 | 200 |
| | 6 | 36 | 2 | 4 | 216 | 0 | 6 | 2 | 10 | 58 |
| | 7 | ND | ND | ND | ND | ND | ND | ND | ND | — |
| | 8 | 423 | 108 | 324 | 389 | 229 | 186 | 51 | 420 | 444 |
| | 9 | 25 | 31 | 21 | 5 | ND | ND | ND | ND | 34 |
| | 10 | 419 | 398 | 331 | 364 | 337 | 331 | 455 | 508 | 655 |
| | 11 | 180 | 130 | 24 | 30 | 21 | 38 | 51 | 16 | 102 |
| | 12 | 433 | 353 | 591 | 283 | 375 | 376 | 380 | 682 | 724 |
| | Mean | | | | | | | | | 264.3 |
| | SD | | | | | | | | | 261.2 |
| Alphagan | 49 | 461 | 465 | 537 | 483 | 460 | 467 | 256 | 196 | 693 |
| 2 mg/mL | 50 | 455 | 417 | 422 | 406 | 426 | 429 | 307 | 242 | 647 |
| | 51 | 291 | 308 | 342 | 343 | 287 | 284 | 278 | 164 | 479 |
| | 52 | 479 | 438 | 504 | 471 | 365 | 456 | 265 | 375 | 699 |
| | 53 | 640 | 653 | 738 | 423 | 657 | 536 | 465 | 344 | 928 |
| | 54 | 160 | 216 | 308 | 322 | 331 | 371 | 266 | 183 | 449 |
| | 55 | ND | ND | ND | ND | ND | ND | ND | ND | — |
| | 56 | 428 | 508 | 338 | 371 | 380 | 564 | 552 | 538 | 766 |
| | 57 | 186 | 201 | 133 | 318 | 308 | 337 | 119 | 145 | 364 |
| | 58 | 411 | 72 | 291 | 205 | 205 | 47 | 259 | 245 | 361 |
| | 59 | 362 | 293 | 48 | 140 | 322 | 231 | 269 | 223 | 393 |
| | 60 | 511 | 437 | 428 | 265 | 304 | 349 | 293 | 191 | 579 |

| | |
|---|---|
| Mean | 578.0 |
| SD | 185.4 |

ND = not determined

TABLE 21
Non-Injured eye RGCs density

Alive cells
(BrN3A positive)
in left eye

| | | | individual |
|---|---|---|---|
| | | cells/photo | mean |
| Treatment | rat id | Value 1 | cells/mm² |
| MANF | 13 | 1 282 | 2 137 |
| 0.15 mg/mL | 14 | 824 | 1 373 |
| MANF | 37 | 1 326 | 2 210 |
| 0.5 mg/mL | 38 | 1 579 | 2 632 |
| MANF | 25 | 1 409 | 2 348 |
| 1.5 mg/mL | 26 | 1 208 | 2 013 |
| PBS | 1 | 890 | 1 483 |
| | 2 | 1 211 | 2 018 |
| Alphagan ® | 49 | 1 718 | 2 127 |
| 2 mg/mL | 50 | 1 718 | 2 863 |

| | |
|---|---|
| mean | 2 120.5 |
| SD | 454.5 |

### CONCLUSION

in these experimental conditions, after a one-week reperfusion period in a rat model of retinal ischemia by clamping, it can be stated that a single intravitreal administration of MANF at 0.15 mg/mL, 0.5 mg/mL and 1.5 mg/mL displayed a marked efficacy in rescuing retinal function (b-wave) and protecting RGC.

The reference Alphagan® (1 mg/kg brimonidine) showed a significant efficacy in improving retinal function and protecting RGC.

### Example 3: 15-Day Ocular Tolerance Study in Rabbits

The aim of this study was to evaluate the ocular tolerance of MANF (3 mg/mL) after a single intravitreal administration (100 μL) in pigmented rabbits over a 15 day period. Ten female pigmented rabbits were divided into two groups of five animals each, corresponding to both treatments. MANF and the vehicle (PBS, pH 7.4) were dosed by intravitreal injection in the right eye once, on Day 1.

The eyes of the animals were examined by Split-lamp and assessed by using the McDonald-Shadduck's scale at Baseline and at Days 1, 3, 8 and 15. At the end of the in-life period on Day 15 both eyeballs of all animals were collected and processed for histology and microscopic examination.

There were no treatment- or administration-related effects on body weight, clinical observations or ophthalmic examinations.

No pathological findings related to treatment were found in any of the eyes observed during histopathology evaluation.

### CONCLUSION

Under the experimental conditions, a single intravitreal administration of MANF in pigmented rabbits was macroscopically and microscopically well tolerated.

### Example 4: Synergistic Effects of MANF Family Proteins and Other Active Agents

In this example, synergy between MANF family proteins and other active agents will be assessed by measuring retinal function after transient vascular clamping of the optic nerve, essentially as described in Example 2. The functional status of the retina will be monitored by electroretinogram (ERG). The b-wave, which is induced by potassium eflux shunted “on” from bipolar cells by Muller cells in response to illumination, is the ERG-component most susceptible to ischemia. Thus, suppression of the b-wave of the ERG has been taken as an electrophysiological measure of retinal blood flow in humans and in experimental animal models. Retinal protection in this model will also be assessed directly by counting RGCs stained with BrN3a.

The MANF family protein (e.g., MANF, CDNF, or fragments thereof) will be administered by intravitreal injection. One or more other active agents (e.g., a prostaglandin analog; a beta-adrenergic receptor antagonist; an alpha adrenergic agonist, e.g., brimonidine; a miotic agent; a carbonic anhydrase inhibitor, etc.) will also be administered. The other active agents may be formulated with the MANF family protein or may be administered separately, by the same or a different route of administration.

The results will show a greater than additive effect. The greater than additive effect may be demonstrated by retinal ganglion cell survival, recovery of B-wave amplitude, or both.

### Example 5: Prophylactic Use of MANF Family Proteins

A MANF family protein (e.g., MANF, CDNF, or a fragment thereof, will be administered periodically (e.g., every: 1-8 weeks, 3-6 weeks, daily) to subjects to prevent retinal ganglion cell loss in the event of an ischemia of the retina, or to reduce the incidence of ischemic events in the retina. The subjects may be animals from an animal model prone to formation of emboli or thrombi.

### REFERENCES, EACH OF WHICH IS INCORPORATED BY REFERENCE IN ITS ENTIRETY

Airavaara M, Shen H, Kuo C C, et al. Mesencephalic astrocyte-derived neurotrophic factor (MANF) reduces ischemic brain injury and promotes behavioral recovery in rats. J Comp Neurol. 2009; 515(1):116-124. Doi: 10.1002/cne.22039.
Airavaara M, Chiocco M, Howard D. et al. Widespread cortnical expression of MANF by rAAV serotype 7: localization and protection against ischemic brain injury. Exp. Neurol 2010; 225(1): 104-113. doi: 10.1016/j.expneurol.2010.05.020.
Apostolou A, Shen Y, Liang Y, et al. Armet, a UPR-upregulated protein, inhibits cell proliferation and ER stress-induced cell death. Exp Cell Res. 2008; 314(13):2454-67.
Barnett N L, Osborne N N. Prolonged bilateral carotid artery occlusion induces electrophysiological and immunohistochemical changes to the rat retina without causing histological damage. Exp Eye Res. 1995; 61(1): 83-90.
Biousse V. Thrombolysis for acute central retinal artery occlusion: is it time? Am J Ophthalmol. 2008; 146(5):631-634.
Bradvica M, Benasic T, Vinkovik M. Retinal vascular occlusions. Advances in Ophthalmology. Dr. Shirnon Rumelt (Ed.) 2012. ISBN: 978-953-51-0248-9. InTech.
Budak V and Akdogan M. Retinal ganglion cell death. Glaucoma—Basic and Clinical Concepts. Dr. Shimon Rumelt (Ed.) 2011, ISBN: 978-953-307-591-4. InTech.
Chen S T, Hsu C Y, Hogan E L, Maricq H, Balentine J D. A model of focal ischemic stroke in the rat: reproducible extensive cortical infarction. Stroke 1986; 17(4):738-743.
Cugati S, Varma D D, Chen C S, and Lew A W. Treatment options for central retinal artery occlusion. Current Treatmnent Options in Neurology 2013; 15:63-77.
Glembotski C C, Thuerauf D J, Huang C, et al. Mesencephalic astrocyte-derived neurotrophic factor protects the heart from ischemic damage and is selectively secreted upon sarco/endoplasmic reticulum calcium depletion. J Biol Chem. 2012; 287(31): 25893-904.
Gorbatyuk M and Gorbatyuk O. Review: Retinal degeneration: focus on the unfolded protein response. Molecular Vision. 2013; 19:1985-1998.
Hayreh S S, Zinunerman M B, Kinmura A, Sanon A. Central retinal artery occlusion. Retinal survival time. Exp. Eye. Res 2004; 78:723-736.
Hayreh S S, Podhajsky P A, Zimmerman B. Retinal artery occlusion: Associated systemic and ophthalmic abnormalities. Ophthalmology 2009; 116(10): 1928-1936.
Hayreh S S. Ocular vascular occlusive disorders: Natural history of visual outcome. Progress in Retinal and Eye Research 2014; 41:1-25.
Helhnan M, Arumnae U, Yu Ly, et al. Mesencephalic astrocyte-derived neurotrophic factor (MANF) has a unique mechanism to rescue apoptotic neurons. Journal of Biological Chemistry. 2011; 286(4):2675-2680.
Hoseki J, Sasakawa H, Yamagu chi Y, et al. Solution structure and dynamics of mouse ARMET. FEBS Lett. 2010; 584(8): 1536-42.
Hu Y, Park K K, Yan Li, et al. Differential effects of unfolded protein response pathways on axon injury-induced death of retinal ganglion cells. Neuron 2012:73:445-452.
Kaur C, Foulds W S, Ling E-A. Hypoxia-ischemia and retinal ganglion cell damage. Clinical Ophthalmology 2008; 2(4):879-889.
Kroeger H, Messah C, Abern K, et al. Induction of endoplasmic reticulum stress genes, BiP and Chop, in genetic and environmental models of retinal degeneration. IOVS. 2012; 53(12):7590-7599.
Leavitt J A, Larson T A, Hodge D O, Gullerud R E. The incidence of central retinal artery occlusion in Olmsted County, Minnesota. Am J Ophthalmnol. 2011; 152(5):820-3.e2.
Lee A-H, Iwakoshi N N, Glimcher L H. XBP-1 regulates a subset of endoplasmic reticulum resident chaperone genes in the unfolded protein response. Mol Cell Biol. 2003; 7448-7459.
Lindholm P, Saarma M. Novel CDNF/MANF family of neurotropic factors. Dev Neurobiol. 2010; 70(5):360-71.
Mizobuchi N, Hoseki J, Kubota H, et al. ARMET is a soluble E R protein induced by the unfolded protein response via ERSE-II element. Cell Struct Funct. 2007; 32(1):41-50.
Nadal-Nicotis F M, Jimnnez-López M, Sobrado-Caivo P, et al. Bm3a as a marker of retinal ganglion cells: qualitative and quantitative time course studies in naive and optic nerve-injured retinas. Invest Ophthalmol Vis Sci. 2009; 50(8):3860-8.
Oh-Hashi K, Tanaka K, Koga H, et al. Intracellular trafficking and secretion of mouse mesencephalic astrocyte-derived neurotrophic factor. Mol Cell Biochem. 2012; 363(1-2): 35-41.
Parkash V, Lindholm P, Perminen J, et al. The structure of the conserved neurotrophic factors MANF and CDNF explains why they are bifunctional. Protein Eng Des Sel. 2009; 22(4):233-41.
Petrova P, Raibekas A, Pevsner J, et al. MANF: a new mesencephalic, astrocyte-derived neurotrophic factor with selectivity for dopamrinergic neurons. J Mol Neurosci. 2003; 20(2):173-88.
Shen Y, Sun A, Wang Y, et al. Upregulation of finesencephalic astrocyte-derived neurotrophic factor in glial cells associated with ischemia-induced glial activation. J Neuroinflanmmrnation 2012; 9(1):254 (1-13).
Silla T, Haal I, Geimanen J, et al. Episomal maintenance of plasmids with hybrid origins in mouse cells. Journal of Virology. 2005; 79(24): 15277-15288.
Tadimalla A, Belmont P J, Thuerauf D J, et al. Mesencephalic astrocyte-derived neurotrophic factor is an ischemia-inducible secreted endoplasmic reticulum stress response protein in the heart. Cire Res. 2008; 103(11):1249-58.
Varma D D, Cugati S, Lee A W and Chen C S. A review of central retinal artery occlusion: Clinical presentation and management. Eye 2013; 27(6):688-697.
Varma D D, Lee A W, Chen C S. Central retinal artery occlusion: General overview, diagnosis, and treatment. Retinal Physician 2013; 10:24-29.
Yang L, Wu L, Guo X and Tso M. Activation of endoplasmic reticulum stress in degenerating photoreceptors of the rdl mouse. IOVS. 2007; 48(11):5191-5198.
Yu Y Q, Liu L C, Wang F C, et al. Induction profile of MANF/ARMET by cerebral ischemia and its implication for neuron protection. Cereb Blood Flow Metab. 2010; 30(1):79-91.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of increasing retinal tolerance time, reducing cell death during an ischemic event in the retina, reducing cell death following an ischemic event in the retina, treating an ischemic event in the retina, or a combination thereof, the method comprising:
(a) administering a dose of a pharmaceutical composition comprising an effective amount of a MANF family protein to a subject in need thereof;
(b) performing a treatment to resolve a blockage causing the ischemic event.

2. The method of claim 1, wherein the MANF family protein is a mesencephalic astrocyte derived neurotrophic factor (MANF) protein, or a fragment thereof.

3. The method of claim 1, wherein the MANF family protein comprises a sequence that has at least about 80% identity with SEQ ID NO:3.

4. The method of claim 1, wherein the MANF family protein comprises a sequence that has 95% identity with SEQ ID NO:3.

5. The method of claim 1, wherein the MANE family protein is a conserved dopamine neurotrophic factor (CDNF) protein, or a fragment thereof.

6. The method of claim 1, wherein the MANF family protein comprises a sequence that has at least about 80% identity with SEQ ID NO:6.

7. The method of claim 1, wherein the MANF family protein comprises a sequence that has 95% identity with SEQ ID NO:6.

8. The method of any one of claims 1-7, wherein the pharmaceutical composition is administered to an eye of the subject.

9. The method of claim 8, wherein the pharmaceutical composition is administered by topical administration, intravitreal injection, intracameral administration, subconjunctival administration, subtenon administration, retrobulbar administration, posterior juxtascleral administration, or a combination thereof.

10. The method of claim 8, wherein the pharmaceutical composition is administered by intravitreal injection.

11. The method of any one of claims 1-10, wherein the dose has a volume of about 25 μL to about 150 μL.

12. The method of any one of claims 1-11, wherein the dose has a concentration of the MANF family protein that is from about 1 mg/mL to about 20 mg/mL.

13. The method of any one of claims 1-1, wherein the dose has a concentration of the MANF family protein that is from about 2.7 mg/mL to about 5.4 mg/mL.

14. The method of any one of claims 1-13, wherein the effective amount of the MANF family protein is from about 50 μg to about 1000 μg.

15. The method of any one of claims 1-13, wherein the effective amount of the MANF family protein is from about 250 μg to about 300 μg.

16. The method of any one of claims 1-15, wherein the dose is administered once every 2 to 8 weeks.

17. The method of any one of claims 1-15, wherein the dose is administered once every 2 to 4 hours.

18. The method of any one of claims 1-15, wherein the dose is only administered once.

19. The method of any one of claims 1-18, wherein the ischemic event is a retinal artery occlusion.

20. The method of any one of claims 1-18, wherein the ischemic event is an acute retinal artery occlusion.

21. The method of any one of claims 1-20, wherein the treatment to resolve the blockage comprises administration of a vasodilator.

22. The method of any one of claims 1-21, wherein the treatment to resolve the blockage comprises ocular massage, intravenous acetazolamide, intravenous mannitol, topical antiglaucoma drops, anterior chamber paracentisis, or a combination thereof.

23. The method of any one of claims 1-22, wherein the treatment to resolve the blockage comprises intravenous methylprednisolone.

24. The method of any one of claims 1-23, wherein the treatment to resolve the blockage comprises Nd YAG laser treatment, pars plana vitrectomy, or a combination thereof.

25. The method of any one of claims 1-24, wherein the treatment to resolve the blockage comprises intravenous tissue plasminogen activator, intra-arterial tissue plasminogen activator, or a combination thereof.

26. The method of any one of claims 1-25, wherein the treatment to resolve the blockage comprises panretinal photocoagulation.

27. The method of any one of claims 1-26, wherein the treatment to resolve the blockage comprises administration of a steroid.

28. The method of any one of claims 1-27, further comprising diagnosing the ischemic event.

29. A method of increasing retinal tolerance time, reducing cell death during a retinal artery occlusion, reducing cell death following a retinal artery occlusion, treating a retinal artery occlusion, or a combination thereof, the method comprising administering a dose of a pharmaceutical composition comprising an effective amount of a MANE family protein to a subject exhibiting one or more symptoms of a retinal artery occlusion.

30. The method of claim 29, wherein the MANF family protein is a mesencephalic astrocyte derived neurotrophic factor (MANF) protein, or a fragment thereof.

31. The method of claim 29, wherein the MANF family protein comprises a sequence that has at least about 80% identity with SEQ ID NO:3.

32. The method of claim 29, wherein the MANF family protein comprises a sequence that has 95% identity with SEQ ID NO:3.

33. The method of claim 29, wherein the MANF family protein is a conserved dopamine neurotrophic factor (CDNF) protein, or a fragment thereof.

34. The method of claim 29, wherein the MANF family protein comprises a sequence that has at least about 80% identity with SEQ ID NO:6.

35. The method of claim 29, wherein the MANE family protein comprises a sequence that has 95% identity with SEQ ID NO:6.

36. The method of any one of claims 29-35, wherein the pharmaceutical composition is administered to an eye of the subject.

37. The method of claim 36, wherein the pharmaceutical composition is administered by topical administration, intravitreal injection, intracameral administration, subconjunctival administration, subtenon administration, retrobulbar administration, posterior juxtascleral administration, or a combination thereof.

38. The method of claim 36, wherein the pharmaceutical composition is administered by intravitreal injection.

39. The method of any one of claims 29-38, wherein the dose has a volume of about 25 μL to about 150 μL.

40. The method of any one of claims 29-39, wherein the dose has a concentration of the MANE family protein that is from about 1 mg/mL to about 20 mg/mL.

41. The method of any one of claims 29-39, wherein the dose has a concentration of the MANF family protein that is from about 2.7 mg/mL to about 5.4 mg/mL.

42. The method of any one of claims 29-41, wherein the effective amount of the MANE family protein is from about 50 μg to about 1000 μg.

43. The method of any one of claims 29-41, wherein the effective amount of the MANF family protein is from about 250 μg to about 300 μg.

44. The method of any one of claims 29-43, wherein the dose is administered once every 2 to 4 hours.

45. The method of any one of claims 29-43, wherein the dose is only administered once.

46. The method of any one of claims 29-45, wherein the retinal artery occlusion is an acute retinal artery occlusion.

47. The method of any one of claims 29-46, wherein the retinal artery occlusion is a central retinal artery occlusion.

48. The method of any one of claims 29-46, wherein the retinal artery occlusion is a branch retinal artery occlusion.

49. A method of treating a retinal disorder, the method comprising administering to a subject in need thereof an effective amount of a MANF family protein and another active agent.

50. The method of claim 49, wherein the MANF family protein and the another active agent have a synergistic effect upon retinal ganglion cell survival.

51. The method of any one of claims 49-50, wherein the MANF family protein and the another active agent exhibit therapeutic synergy.

52. The method of any one of claims 49-51, wherein the MANF family protein is MANF, or a fragment thereof.

53. The method of any one of claims 49-52, wherein the MANF family protein is CDNF, or a fragment thereof.

54. The method of any one of claims 49-53, wherein the another active agent is a prostaglandin analog, a beta-adrenergic receptor antagonist, an alpha adrenergic agonist, a miotic agent, a carbonic anhydrase inhibitor, or a combination thereof.

55. The method of any one of claims 49-54, wherein the another active agent is brimonidine or a pharmaceutical salt thereof.

56. The method of any one of claims 49-55, wherein the retinal disorder is an acute retinal artery occlusion.

57. The method of any one of claims 49-55, wherein the retinal disorder is a central retinal artery occlusion or a branch retinal artery occlusion.

58. The method of any one of claims 49-55, wherein the retinal disorder is retinal ischemia.

59. The method of any one of claims 49-55, wherein the retinal disorder is macular degeneration, diabetic eye disease, age-related macular degeneration, branch retinal vein occlusion, central retinal vein occlusion, central retinal artery occlusion, central serous retinopathy, diabetic retinopathy, Fuchs' dystrophy, giant cell arteritis, glaucoma, hypertensive retinopathy, thyroid eye disease, iridocorneal endothelial syndrome, ischemic optic neuropathy, juvenile macular degeneration, macular edema, macular telangioctasia, marfan syndrome, optic neuritis, photokeratitis, retinitis pigmentosa, retinopathy of prematurity, stargardt disease, usher syndrome, or any combination thereof.

60. The method of any one of claims 49-59, wherein administration of the MANF family protein is topical, subconjunctival, intravitreal, retrobulbar, intracameral, systemic, or a combination thereof.

61. The method of any one of claims 49-60, wherein the effective amount of the MANF family protein is at least about: 0.5 μg, 2.5 μg, 5 μg, 7.5 μg, 12.5 μg, 25 μg, 50 μg, 75 μg, 100 μg, 150 μg, 250 μg, 500 μg, 1000 μg, 1250 μg, or 2500 μg per eye.

62. The method of any one of claims 49-61, wherein the MANF family protein is administered once every 2 to 8 weeks.

63. The method of any one of claims 49-61, wherein the MANF family protein is administered only once.

64. A pharmaceutical composition comprising an amount of a MANF family protein and another active agent that is effective for treating a retinal disorder.

65. The pharmaceutical composition of claim 64, wherein the MANF family protein and the another active agent have a synergistic effect upon retinal ganglion cell survival.

66. The pharmaceutical composition of any one of claims 64-65, wherein the MANF family protein and the another active agent exhibit therapeutic synergy.

67. The pharmaceutical composition of any one of claims 64-66, wherein the MANF family protein is MANF, or a fragment thereof.

68. The pharmaceutical composition of any one of claims 64-67, wherein the MANF family protein is CDNF, or a fragment thereof.

69. The pharmaceutical composition of any one of claims 64-68, wherein the another active agent is a prostaglandin analog, a beta-adrenergic receptor antagonist, an alpha adrenergic agonist, a miotic agent, a carbonic anhydrase inhibitor, or a combination thereof.

70. The pharmaceutical composition of any one of claims 64-69, wherein the another active agent is brimonidine or a pharmaceutical salt thereof.
